(19) United States
(12) Patent Application Publication
(10) Pub. No.: US 2011/0118261 A1
(43) Pub. Date: May 19, 2011

(54) BIS-PYRIDYLPYRIDONES AS MELANIN-CONCENTRATING HORMONE RECEPTOR 1 ANTAGONISTS

(75) Inventors: Scott Allen, Durham, NC (US); William C. Blackwell, III, Durham, NC (US); Eric Boros, Durham, NC (US); Jon L. Collins, Durham, NC (US); Don Hertzog, Durham, NC (US); Xi Liang, Durham, NC (US); John Ray, Durham, NC (US); Steven Michael Reister, Durham, NC (US); Vicente Samano, Durham, NC (US); Ron Sherrill, Durham, NC (US)

(73) Assignee: Glaxo Group Limited

(21) Appl. No.: 12/747,186

(22) PCT Filed: Dec. 10, 2008
(86) PCT No.: PCT/US2008/086131

§ 371 Date: Jan. 31, 2011

Related U.S. Application Data
(60) Provisional application No. 61/012,525, filed on Dec. 10, 2007.

(51) Int. Cl. A61K 31/5377; C07D 401/14; C07D 413/14; C07D 405/14; A61K 31/444; A61K 31/4545; A61P 3/10; A61P 3/04; A61P 9/12; A61P 25/24; A61P 25/22; A61P 25/30

(52) U.S. Cl. 514/235.5; 546/256; 544/131; 546/193; 514/333; 514/318

(57) ABSTRACT

The invention provides novel bis-pyridylpyridones which are antagonists at the melanin-concentrating hormone receptor 1 (MCHR1), pharmaceutical compositions containing them, processes for their preparation, and their use in therapy and for the treatment of obesity and/or diabetes.

### FIELD OF INVENTION

This invention relates to novel bis-pyridylpyridones which are antagonists at the melanin-concentrating hormone receptor 1 (MCHR1), to pharmaceutical compositions containing them, to processes for their preparation, and to their use in therapy for the treatment of obesity and/or diabetes.

### BACKGROUND OF THE INVENTION

Obesity is a medical condition that is reaching epidemic proportions among humans in a number of countries throughout the world. It is a condition that is also associated with or induces other diseases or conditions that disrupt life activities and lifestyles. Obesity is recognized as a serious risk factor for other diseases and conditions such as diabetes, hypertension, and arteriosclerosis. It is also known that increased body weight due to obesity can place a burden on joints, such as knee joints, causing arthritis, pain, and stiffness.

Because overeating and obesity have become such a problem in the general population, many individuals are now interested in losing weight, reducing weight, and/or maintaining a healthy body weight and desirable lifestyle.

It is known that melanin-concentrating hormone originates in the hypothalamus and has orexigenic action (see Nature, Vol. 396, p. 670 (1998), for example. There is an on-going need for the development of a melanin-concentrating hormone antagonist useful in the treatment of obesity and other associated or related diseases and conditions.

Accordingly, we have now found a novel group of bis-pyridylpyridones that exhibit a useful profile of activity as antagonists of the melanin-concentrating hormone receptor (MCHR1).

### SUMMARY OF THE INVENTION

The present invention provides a compound of Formula I,

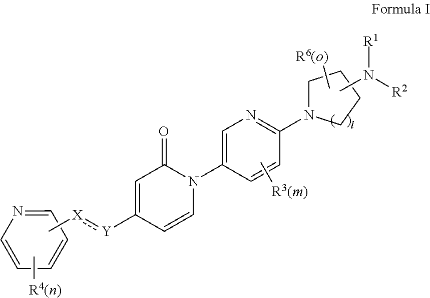

or salt thereof, wherein:
X and Y are independently selected from the group consisting of —O—, —CH₂—, and ═CH—, with the proviso that X and Y are not both —O—;

— is optionally a bond to form a double bond;

R¹ is selected from the group consisting of (i) hydrogen, (ii) substituted or unsubstituted, straight or branched C₁₋₆alkyl, and (iii) substituted or unsubstituted C₃₋₆cycloalkyl;

R² is selected from the group consisting of (i) hydrogen, (ii) substituted or unsubstituted, straight or branched C₁₋₆alkyl, (iii) —C(O)NH₂, (iv) —C(O)R⁵, (v) −SO₂R⁵, and (vi) C(O)OR¹;

or R¹ and R² together with the nitrogen to which they are attached to form a heterocycle, and said heterocycle is optionally substituted with one, two, or three R⁵ groups;

wherein each R⁵ independently is selected from the group consisting of (i) hydroxy, (ii) unsubstituted or substituted C₁₋₃alkoxy, (iii) unsubstituted or substituted, straight or branched C₁₋₆alkyl, and (iv) unsubstituted or substituted C₃₋₆cycloalkyl;

each R³ and R⁴ independently is selected from the group consisting of H, F, Cl, CF₃, CH₃, CH₂CH₃, CH₂CF₃, cyclopropyl, OMe, OEt, OiPr, O-cyclopropyl, OCF₃, OCH₂CF₃, CN, NMe₂, N-pyrrolidinyl, N-morpholinyl, and acetyl;

R⁶ is selected from the group consisting of (i) hydrogen, (ii) substituted or unsubstituted, straight or branched C₁₋₆alkyl, and (iii) substituted or unsubstituted C₃₋₆cycloalkyl;

l is 0, 1, or 2;

m is 0, 1, 2, or 3;

n is 0, 1, 2, or 3; and

o is 0, 1, 2, or 3.

There is also provided a pharmaceutical composition comprising a compound of Formula I or salt thereof.

Further, there is provided a pharmaceutical composition comprising a compound of Formula I or salt thereof and one or more excipients.

There is still further provided a method of treatment comprising the administering to a mammal, particularly a human, a pharmaceutical composition comprising a compound of Formula I or pharmaceutically acceptable salt thereof and at least one excipient, wherein said treatment is for obesity, diabetes, depression, or anxiety.

Additionally, there is provided a compound of Formula I or pharmaceutically acceptable salt thereof for use as an active therapeutic substance (in therapy).

And, there is also provided a compound of Formula I or pharmaceutically acceptable salt thereof for use in the treatment of obesity, diabetes, depression, or anxiety in a mammal, especially a human.

A process for preparing a compound of Formula I or pharmaceutically acceptable salt thereof is also provided.

### DETAILED DESCRIPTION OF THE INVENTION

The present invention provides a compound of Formula I,

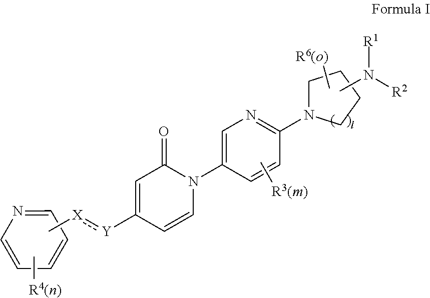

or salt thereof.

In Formula I, X and Y are joined by a single bond or a double bond (depicted in the structure as “—”). Preferably, X and Y are joined by a single bond. X and Y are joined by a double bond only when both are ═CH—. X and Y cannot both be —O—. Each X and Y independently are selected from the group consisting of —O—, —CH₂—, and ═CH—. —XY can be, for example, —CH₂—CH₂—, —O—CH₂—, —CH₂—O—, and —HC═CH—. Preferably, each X and Y is independently selected from the group consisting of —O— and —CH₂—. That is, X and Y together are —CH₂O— or —OCH₂—.

R¹ of Formula I is selected from the group consisting of (i) hydrogen, (ii) substituted or unsubstituted, straight or branched C₁₋₆ alkyl, and (iii) substituted or unsubstituted C₃₋₆ cycloalkyl. Preferably, when R¹ is a substituted C₁₋₆alkyl or a substituted C₃₋₆cycloalkyl, it is substituted with one to six fluorines (F).

R² of Formula I is selected from the group consisting of (i) hydrogen, (ii) substituted or unsubstituted, straight or branched, C₁₋₆ alkyl, (iii) —C(O)NH₂, (iv) —C(O)R⁵, (v) —SO₂R⁵, and (vi) —C(O)OR¹. When R² is a substituted C₁₋₆ alkyl, preferably it is substituted with one to six fluorines. In one embodiment, R¹ and R² are, respectively, a hydrogen and an ethyl group.

In Formula I, R¹ and R² can be joined together along with the nitrogen to which they are attached to form a heterocycle. The heterocycle is optionally and preferably substituted with one, two, or three R⁵ groups. Preferably, R¹ and R² are joined together with the nitrogen to which they are attached to form a pyrrolidinyl, piperidinyl, piperazinyl optionally substituted on N′ with a R², or a morpholinyl group.

R⁵ is selected from the group consisting of (i) hydroxy, (ii) unsubstituted or substituted C₁₋₃ alkoxy, (iii) unsubstituted or substituted, straight or branched C₁₋₆ alkyl and (iv) unsubstituted or substituted C₃₋₆cycloalkyl. When R³ is a substituted C₁₋₃alkoxy, substituted C₁₋₆alkyl, or substituted C₃₋₆cycloalkyl, it can be substituted with one to six fluorines.

R⁶ is selected from the group consisting of (i) hydrogen, (ii) substituted or unsubstituted, straight or branched C₁₋₆alkyl, and (iii) substituted or unsubstituted C₃₋₆cycloalkyl.

In Formula I, each R³ and R⁴ independently is selected from the group consisting of H, F, Cl, CF₃, CH₃, CH₂CH₃, CH₂CF₃, cyclopropyl, OMe, OEt, OiPr, O-cyclopropyl, OCF₃, OCH₂CF₃, CN, NMe₂, N-pyrrolidinyl, N-morpholinyl, and acetyl.

In Formula I, l is 0, 1, or 2. This means that the ring in which l is located can contained 4, 5, or 6 ring atoms. Preferably, l is 1 or 2, most preferably 1.

In Formula I, m is 0, 1, 2, or 3; preferably m is 0, 1, or 2.

In Formula I, n is 0, 1, 2, or 3; preferably n is 0, 1, or 2.

In Formula I, o is 0, 1, 2, or 3; preferably o is 0, 1, or 2.

Preferred compounds of the invention are
4-{[(5-chloro-2-pyridinyl)methyl]oxy}-6′-[3-(ethylamino)-1-pyrrolidinyl]-2H-1,3′-bipyridin-2-one;
4-{[(5-chloro-2-pyridinyl)methyl]oxy}-6′-[3-(methylamino)-1-pyrrolidinyl]-2H-1,3′-bipyridin-2-one;
4-{[(5-chloro-2-pyridinyl)methyl]oxy}-6′-[3-(dimethylamino)-1-pyrrolidinyl]-2H-1,3′-bipyridin-2-one;
4-{[(5-chloro-2-pyridinylmethyl]oxy}-6′-[3-(dimethylamino)-1′-pyrrolidinyl]-5′-methyl-2H-1,3′-bipyridin-2-one;
4-{[(5-chloro-2-pyridinylmethyl]oxy}-6′-[3-(propylamino)-1-pyrrolidinyl]-2H-1,3′-bipyridin-2-one;
4-{[(5-chloro-2-pyridinylmethyl]oxy}-6′-{3-[ethyl(methyl)amino]-1-pyrrolidinyl}-2H-1,3′-bipyridin-2-one;
4-{[(5-chloro-2-pyridinylmethyl]oxy}-6′-{3-[methyl(1-methylethyl)amino]-1-pyrrolidinyl}-2H-1,3′-bipyridin-2-one;
4-{[(5-chloro-2-pyridinylmethyl]oxy}-6′-[3-(cyclohexylamino)-1-pyrrolidinyl]-2H-1,3′-bipyridin-2-one;
4-{[(5-chloro-2-pyridinylmethyl]oxy}-6′-[3-(cyclopentylamino)-1-pyrrolidinyl]-2H-1,3′-bipyridin-2-one;
4-{[(5-chloro-2-pyridinylmethyl]oxy}-6′-(3-{[2-(methyloxy)ethyl]amino}-1-pyrrolidinyl)-2H-1,3′-bipyridin-2-one;
4-{[(5-chloro-2-pyridinylmethyl]oxy}-6′-[3-(tetrahydro-2H-pyran-4-ylamino)-1-pyrrolidinyl]-2H-1,3′-bipyridin-2-one;
4-{[(5-chloro-2-pyridinylmethyl]oxy}-6′-(1,3-bipyrrolidin-1′-yl)-2H-1,3′-bipyridin-2-one;
4-{[(5-chloro-2-pyridinylmethyl]oxy}-6′-[3-(4-morpholinyl)-1-pyrrolidinyl]-2H-1,3′-bipyridin-2-one;
4-{[(5-chloro-2-pyridinylmethyl]oxy}-6′-[3-(amino)-1-pyrrolidinyl]-2H-1,3′-bipyridin-2-one;
4-{[(5-chloro-2-pyridinylmethyl]oxy}-6′-[3-(methoxycarbonylamino)-1-pyrrolidinyl]-2H-1,3′-bipyridin-2-one;
4-{[(5-chloro-2-pyridinylmethyl]oxy}-6′-[4-(N-methylamino)-1-piperidinyl]-2H-1,3′-bipyridin-2-one;
4-{[(5-chloro-2-pyridinylmethyl]oxy}-6′-[3-(N-methylacetamido)-1-pyrrolidinyl]-2H-1,3′-bipyridin-2-one;
4-{[(5-chloro-2-pyridinylmethyl]oxy}-6′-[3-(N-methylamino)-4-methyl-1-pyrrolidinyl]-2H-1,3′-bipyridin-2-one;
4-{[(5-fluoro-2-pyridinylmethyl]oxy}-6′-[3-(dimethylamino)-1-pyrrolidinyl]-2H-1,3′-bipyridin-2-one;
4-{[(2-pyridinyl)methyl]oxy}-6′-[3-(dimethylamino)-1-pyrrolidinyl]-2H-1,3′-bipyridin-2-one; or a salt thereof.

Of these, the most preferred compounds are
4-{[(5-chloro-2-pyridinyl)methyl]oxy}-6′-[3-(ethylamino)-1-pyrrolidinyl]-2H-1,3′-bipyridin-2-one;
4-{[(5-chloro-2-pyridinyl)methyl]oxy}-6′-[3-(methylamino)-1-pyrrolidinyl]-2H-1,3′-bipyridin-2-one;
4-{[(5-chloro-2-pyridinyl)methyl]oxy}-6′-[3-(dimethylamino)-1-pyrrolidinyl]-2H-1,3′-bipyridin-2-one;
4-{[(5-chloro-2-pyridinyl)methyl]oxy}-6′-[3-(dimethylamino)-1-pyrrolidinyl]-5′-methyl-2H-1,3′-bipyridin-2-one;
4-{[(5-chloro-2-pyridinyl)methyl]oxy}-6′-[3-(propylamino)-1-pyrrolidinyl]-2H-1,3′-bipyridin-2-one;
4-{[(5-chloro-2-pyridinyl)methyl]oxy}-6′-{3-[ethyl(methyl)amino]-1-pyrrolidinyl}-2H-1,3′-bipyridin-2-one;
4-{[(5-chloro-2-pyridinyl)methyl]oxy}-6′-{3-[methyl(1-methylethyl)amino]-1-pyrrolidinyl}-2H-1,3′-bipyridin-2-one;
4-{[(5-chloro-2-pyridinyl)methyl]oxy}-6′-[3-(cyclohexylamino)-1-pyrrolidinyl]-2H-1,3′-bipyridin-2-one;
4-{[(5-chloro-2-pyridinyl)methyl]oxy}-6′-[3-(cyclopentylamino)-1-pyrrolidinyl]-2H-1,3′-bipyridin-2-one;
4-{[(5-chloro-2-pyridinyl)methyl]oxy}-6′-(3-{[2-(methyloxy)ethyl]amino}-1-pyrrolidinyl)-2H-1,3′-bipyridin-2-one;
4-{[(5-chloro-2-pyridinyl)methyl]oxy}-6′-[3-(tetrahydro-2H-pyran-4-ylamino)-1-pyrrolidinyl]-2H-1,3′-bipyridin-2-one;
4-{[(5-chloro-2-pyridinyl)methyl]oxy}-6′-(1,3′-bipyrrolidin-1′-yl)-2H-1,3′-bipyridin-2-one;
   or a salt thereof.

It will be appreciated by those skilled in the art that the compound of the present invention may also be utilized in the form of a pharmaceutically acceptable salt thereof.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. In general, the salts are formed from pharmaceutically acceptable inorganic and organic acids. More specific examples of suitable acid salts include maleic, hydrochloric, hydrobromic, sulphuric, phosphoric, nitric, perchloric, fumic, acetic, propionic, succinic, glycolic, formic, lactic, aleic, tartaric, citric, palmoic, malonic, hydroxymaleic, phenylacetic, glutamic, benzoic, salicylic, fumaric, toluenesulfonic, methansulfonic (mesylate), naphthaliene-2-sulfonic, benzenesulfonic, hydroxynaphthoic, hydroiodic, malic, teroic, tannic, and the like.

Other representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, oxalate, pamoate (embonate), palmitate, pantothenate, phosphate/diphosphate, polygalacturonate, salicylate, stearate, subacetate, succinate, sulfate, tannate, tartrate, teoclate, tosylate, triethiodide, and valerate salts.

Other salts, which are not pharmaceutically acceptable, may be useful in the preparation of compounds of this invention and these should be considered to form a further aspect of the invention. These salts, such as oxalic or trifluoroacetate, while not in themselves pharmaceutically acceptable, may be useful in the preparation of salts useful as intermediates in obtaining the compounds of the invention and their pharmaceutically acceptable salts.

The compound of Formula I or a salt thereof may exist in stereoisomeric forms (e.g., it contains one or more asymmetric carbon atoms). The individual stereoisomers (enantiomers and diastereomers) and mixtures of these are included within the scope of the present invention. The invention also covers the individual isomers of the compound or salt represented by Formula I as mixtures with isomers thereof in which one or more chiral centers are inverted. Likewise, it is understood that a compound or salt of Formula I may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. Also included within the scope of the invention are individual isomers of the compound represented by Formula I, as well as any wholly or partially equilibrated mixtures thereof. The present invention also includes the individual isomers of the compound or salt represented by the Formula I as well as mixtures with isomers thereof in which one or more chiral centers are inverted. It is to be understood that the present invention includes all combinations and subsets of the particular groups defined hereinabove.

Terms are used within their accepted meanings. The following definitions are meant to clarify, but not limit, the terms defined.

As used herein, the term “alkyl” (or “alkylene”) refers to a straight or branched chain alkyl, preferably having from one to twelve carbon atoms, which may be unsubstituted or substituted, with multiple degrees of substitution included within the present invention. Examples of “alkyl” as used herein include, but are not limited to, methyl, ethyl, propyl, isopropyl, isobutyl, n-butyl, t-butyl, isopentyl, n-pentyl, and the like, as well as substituted versions thereof.

As used herein, the term “cycloalkyl” refers to an unsubstituted or substituted mono- or polycyclic non-aromatic saturated ring, which optionally includes an alkylene linker through which the cycloalkyl may be attached. Exemplary “cycloalkyl” groups include, but are not limited to, cyclopropyl, cyclobutyl, cyclopentyl, cyclohexyl, cycloheptyl, and the like, as well as unsubstituted and substituted versions thereof.

As used herein, the term “alkoxy” refers to the group —OR^{a}, where R^{a} is alkyl or cycloalkyl as defined above.

As used herein, the term “heterocycle” or “heterocyclyl” refers to unsubstituted and substituted mono- or polycyclic non-aromatic ring system containing one or more heteroatoms. Preferred heteroatoms include N, O, and/or S, including N-oxides, sulfur oxides, and dioxides. Preferably the ring is three to eight-membered and is either fully saturated or has one or more degrees of unsaturation. Multiple degrees of substitution are included within the present definition. Examples of “heterocyclic” groups include, but are not limited to piperidinyl, pyrrolidinyl, morpholinyl, azetidinyl, piperazinyl, pyrrolidinonyl, piperazinonyl, pyrazolidinyl, and their various tautomers.

As used herein, the term “cyano” refers to the group —CN.

As used herein, the term “acetyl” refers to the group —C(O)R^{b}, where R^{b} is alkyl, cycloalkyl, or heterocyclyl, as each is defined herein.

As used herein, the term “optionally” means that the subsequently described event(s) may or may not occur, and includes both event(s) that occur and event(s) that do not occur.

As used herein, the phrase “optionally substituted” or variations thereof denote an optional substitution, including multiple degrees of substitution, with one or more substitutent group. The phrase should not be interpreted as duplicative of the substitutions herein described and depicted. Exemplary optional substituent groups or “substituted” as used herein include acyl; alkyl; alkylsulfonyl; alkoxy; alkoxycarbonyl; cyano; halogen; haloalkyl; hydroxyl; oxo; and nitro.

The compounds of this invention may be made by a variety of methods, including well-known standard synthetic methods. Illustrative general synthetic methods are set out below and then specific compounds of the invention are prepared in the working examples.

In all of the schemes described below, protecting groups for sensitive or reactive groups are employed where necessary in accordance with general principles of synthetic chemistry. Protecting groups are manipulated according to standard methods of organic synthesis (T. W. Green and P. G. M. Wuts, (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention. Compounds of the invention can be readily prepared according to Schemes 1 through 3 by those skilled in the art.

As illustrated in Scheme 1, hydroformylation of substituted bromopyridine (A) followed by reduction provided hydroxymethylpyridine intermediate (B). Reaction of intermediate (B) with 4-nitropyridine-1-oxide in the presence of sodium metal provided substituted hydroxymethyl ether intermediate (C). Treatment of intermediate (C) with trifluoroacetic anhydride (TFAA) provided the desired pyridone intermediate (D).

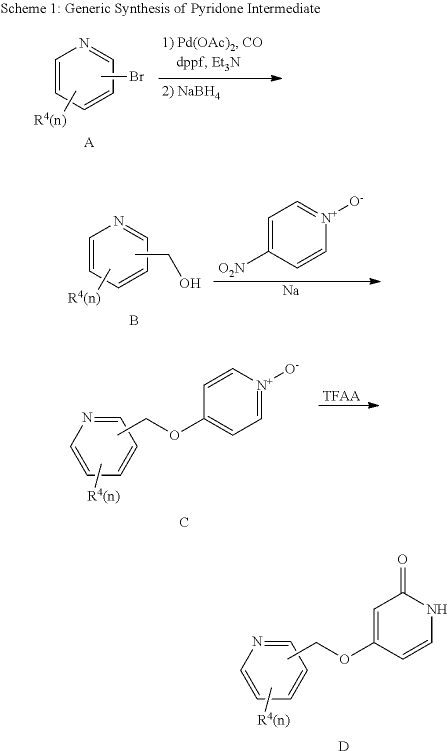

Compounds of the invention can be prepared as illustrated in Scheme 2. Briefly, reaction of substituted pyridone intermediate (D) with 2-aminopyridine intermediate (E) provided 2-aminopyridine-pyridone example (F).

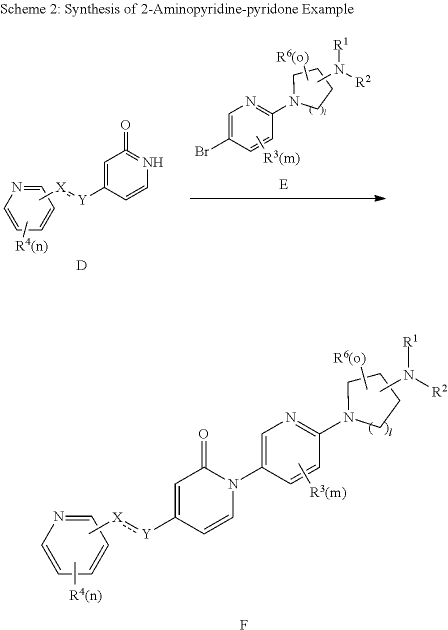

Compounds of the invention can also be prepared as illustrated in Scheme 3. Briefly, heating substituted 2-halo-5-bromopyridine with 3-hydroxypyrrolidine in the presence of base provided hydroxypyrrolidine intermediate (G). Formation of an intermediate mesylate (H) followed by displacement of the mesylate group with substituted amine provided substituted 2-aminopyridine intermediate (E). Copper-mediated coupling of intermediate (E) with substituted pyridone intermediate (D) provided 2-aminopyridine-pyridone example (F). In Scheme 3, MsCl is methanesulfonylchloride, Me is methyl, Et is ethyl, CuI is copper iodide, and NaI is sodium iodide.

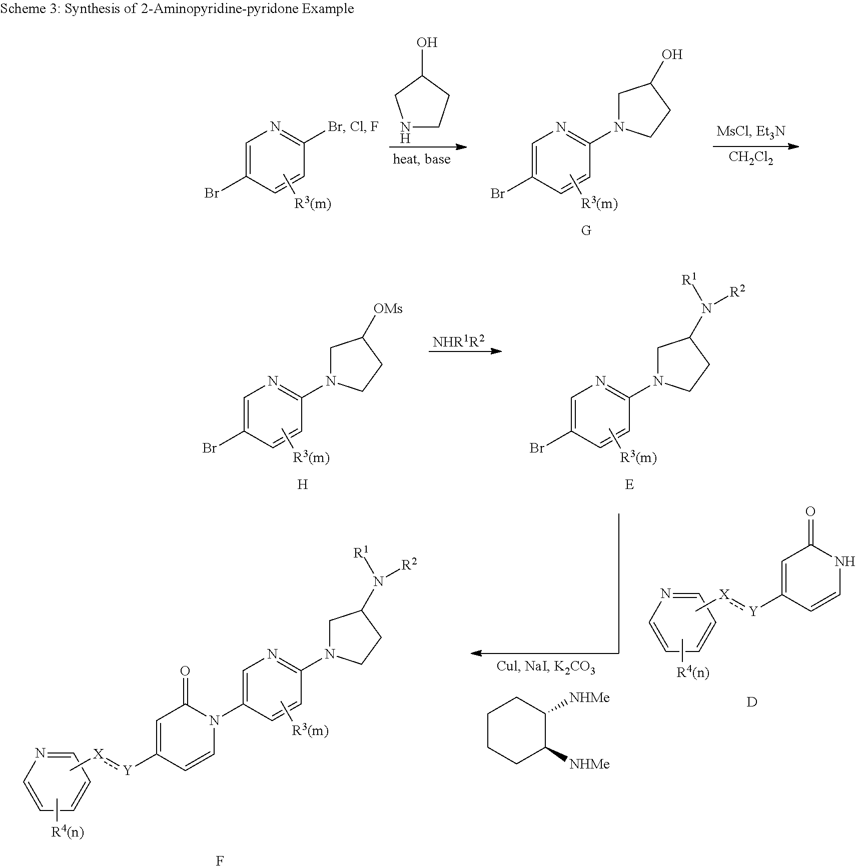

Compounds of the invention can also be prepared as illustrated in Scheme 4. Treatment of substituted 2-halo-5-bromopyridine with substituted piperidine in the presence of base provided substituted aminopyridine intermediate (I). Copper-mediated coupling of intermediate (E) with substituted pyridone intermediate (D) provided 2-aminopyridine-pyridone example (F).

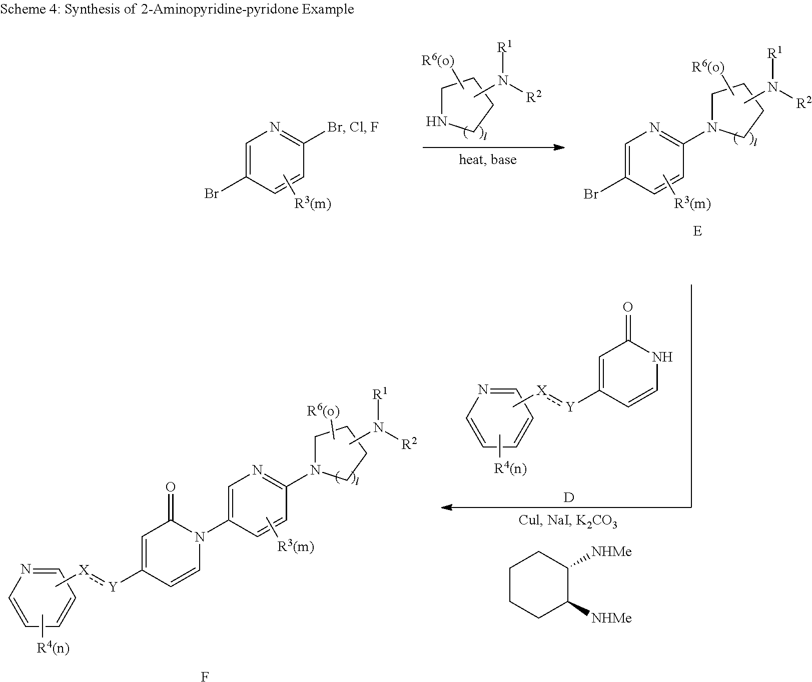

Compounds of the invention can also be prepared as illustrated in Scheme 5. Oxidation of methylisonicotinate followed by treatment with acetic anhydride, then methanol, provided methyl 2-oxo-1,2-dihydro-4-pyridinecarboxylate. Reduction of the ester with LiBH₄ (lithium borohydride), followed by protection of the primary alcohol as the TBDMS ether (tertiarybutyldimethylsilyl ether), provided intermediate (I). Copper-mediated coupling of 2-aminopyridine intermediate (E) with intermediate (I) provided substituted intermediate (J). Acid-catalyzed removal of the silyl protecting group followed by subjection to a Mitsunobu reaction with substituted phenol provided 2-aminopyridine-pyridone example (F). In Scheme 5, MeReO₃ is methyltrioxorhenium, THF is tetrahydrorfuran, DMF is N,N-dimethylformamide, DIAD is diisopropylazodicarboxylate, and TFA is trifluoroacetic acid.

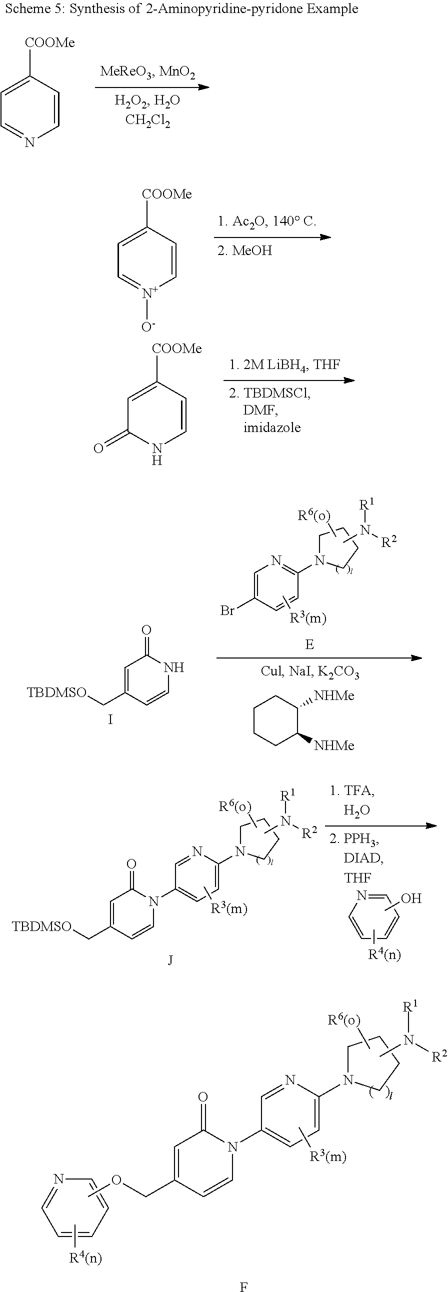

Compounds of the invention can also be prepared as illustrated in Scheme 6. Briefly, reaction of substituted pyridone intermediate (D) with 2-amino-5-halo pyridine (K) provided 2-aminopyridine intermediate (L). Subsequent treatment of such 2-aminopyridine intermediate (L) with HF/Pyridine, followed by treatment with NaNO₂, provided 2-fluoropyridine intermediate (M). Reaction of such 2-fluoropyridine intermediate (M) with the amine encompassed within the scope of this invention provided 2-aminopyridine-pyridone example (F).

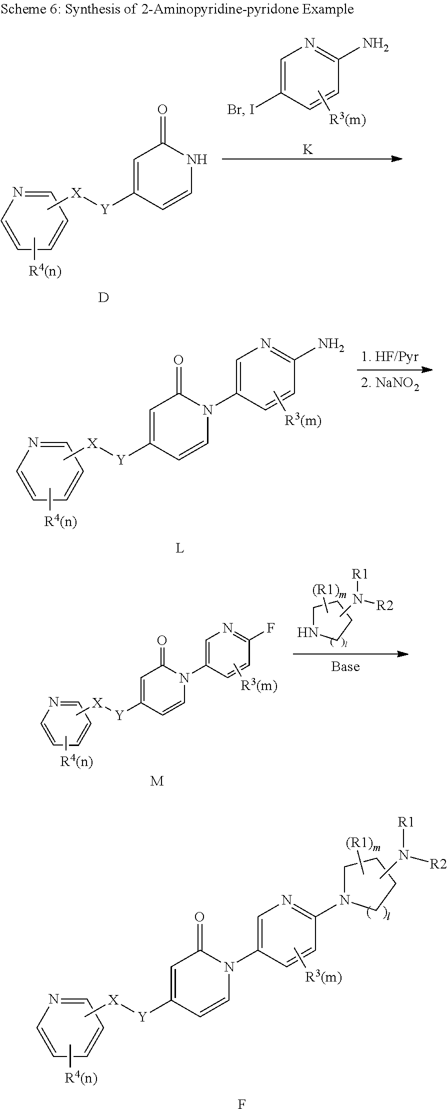

The invention further provides a pharmaceutical composition (also referred to as pharmaceutical formulation) comprising a compound of Formula I or salt, thereof and one or more excipients (also referred to as carriers and/or diluents in the pharmaceutical arts). The excipients are acceptable in the sense of being compatible with the other ingredients of the formulation and not deleterious to the recipient thereof (i.e., the patient).

In accordance with another aspect of the invention there is provided a process for the preparation of a pharmaceutical composition comprising mixing (or admixing) a compound of Formula I or salt thereof with at least one excipient.

Pharmaceutical compositions may be in unit dose form containing a predetermined amount of active ingredient per unit dose. Such a unit may contain a therapeutically effective dose of the compound of Formula I or salt thereof or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well-known in the pharmacy art.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).

When adapted for oral administration, pharmaceutical compositions may be in discrete units such as tablets or capsules; powders or granules; solutions or suspensions in aqueous or non-aqueous liquids; edible foams or whips; oil-in-water liquid emulsions or water-in-oil liquid emulsions. The compound or salt thereof of the invention or the pharmaceutical composition of the invention may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutical carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants such as colloidal silica, talc, magnesium stearate, calcium stearate, solid polyethylene glycol can be added to the powder mixture before the filling operation. A disintegrating or solubilizing agent such as agar-agar, calcium carbonate, or sodium carbonate can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars, such as glucose or beta-lactose, corn sweeteners, natural and synthetic gums such as acacia, tragacanth, sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, and aliginate, gelatin, or polyvinyl pyrrolidone, a solution retardant such as paraffin, a resorption accelerator such as a quaternary salt, and/or an absorption agent such as bentonite, kaolin, or dicalcium phosphate. The powder mixture can be granulated by wetting a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compound or salt of the present invention can also be combined with a free-flowing inert carrier and compressed into tablets directly without going through the granulating or slugging steps. A clear opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving the compound or salt thereof of the invention in a suitably flavoured aqueous solution, while elixirs are prepared through the use of a non-toxic alcoholic vehicle. Suspensions can be formulated by dispersing the compound or salt of the invention in a non-toxic vehicle. Solubilizers and emulsifiers, such as ethoxylated isostearyl alcohols and polyoxyethylene sorbitol ethers, preservatives, flavor additives such as peppermint oil, natural sweeteners, saccharin, or other artificial sweeteners, and the like, can also be added.

Where appropriate, dosage unit formulations for oral administration can be microencapsulated. The formulation can also be prepared to prolong or sustain the release as, for example, by coating or embedding particulate material in polymers, wax, or the like.

In the present invention, tablets and capsules are preferred for delivery of the pharmaceutical composition.

As used herein, the term “treatment” includes prophylaxis and refers to alleviating the specified condition, eliminating or reducing one or more symptoms of the condition, slowing or eliminating the progression of the condition, and preventing or delaying the reoccurrence of the condition in a previously afflicted or diagnosed patient or subject. Prophylaxis (or prevention or delay of disease onset) is typically accomplished by administering a drug in the same or similar manner as one would to a patient with the developed disease or condition.

The present invention provides a method of treatment in a mammal, especially a human, suffering from obesity, diabetes, hypertension, depression, anxiety, drug addiction, substance addiction, or a combination thereof depression. Such treatment comprises the step of administering a therapeutically effective amount of a compound of Formula I or salt thereof to said mammal, particularly a human. Treatment can also comprise the step of administering a therapeutically effective amount of a pharmaceutical composition containing a compound of Formula I or salt thereof to said mammal, particularly a human.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula I, as well as salts thereof, may be administered as the raw chemical. Additionally, the active ingredient may be presented as a pharmaceutical composition.

While it is possible that, for use in therapy, a therapeutically effective amount of a compound of Formula I or salt thereof may be administered as the raw chemical, it is typically presented as the active ingredient of a pharmaceutical composition or formulation.

The precise therapeutically effective amount of a compound or salt thereof of the invention will depend on a number of factors, including, but not limited to, the age and weight of the subject (patient) being treated, the precise disorder requiring treatment and its severity, the nature of the pharmaceutical formulation/composition, and route of administration, and will ultimately be at the discretion of the attending physician or veterinarian. Typically, a compound of Formula I or salt thereof will be given for the treatment in the range of about 0.1 to 100 mg/kg body weight of recipient (patient, mammal) per day and more usually in the range of 0.1 to 10 mg/kg body weight per day. Acceptable daily dosages may be from about 1 to about 1000 mg/day, and preferably from about 1 to about 100 mg/day. This amount may be given in a single dose per day or in a number (such as two, three, four, five, or more) of sub-doses per day such that the total daily dose is the same. An effective amount of a salt thereof may be determined as a proportion of the effective amount of the compound of Formula I per se. Similar dosages should be appropriate for treatment (including prophylaxis) of the other conditions referred herein for treatment. In general, determination of appropriate dosing can be readily arrived at by one skilled in medicine or the pharmacy art.

Additionally, the present invention comprises a compound of Formula I or salt thereof or a pharmaceutical composition thereof with at least one other anti-obesity drug and/or at least one anti-diabetes drug. Such anti-obesity drugs can include, for example, Metformin (or glucophage), CB1 receptor antagonists, GLP-1 agonists, opioid antagonists, and neurotransmitter reuptake inhibitors. When a compound of the invention is employed in combination with another anti-obesity drug or anti-diabetes drug, it is to be appreciated by those skilled in the art that the dose of each compound or drug of the combination may differ from that when the drug or compound is used alone. Appropriate doses will be readily appreciated and determined by those skilled in the art. The appropriate dose of the compound of Formula I or salt thereof and the other therapeutically active agent(s) and the relative timings of administration will be selected in order to achieve the desired combined therapeutic effect, and are with the expertise and discretion of the attending doctor or clinician.

### EXPERIMENTAL

The following examples are intended for illustration only and are not intended to limit the scope of the invention in any way, the invention being defined by the claims. Unless otherwise noted, reagents are commercially available or are prepared according to procedures in the literature. The symbols and conventions used in the descriptions of processes, schemes, and examples are consistent with those used in the contemporary scientific literature, for example, the Journal of the American Chemical society or the Journal of Biological Chemistry. Unless otherwise indicated, all temperatures are expressed in degrees Centigrade. All reactions were conducted at room temperature unless otherwise noted.

### I. Preparation of Intermediates

### Intermediate 1: 1-(5-bromo-2-pyridinyl)-3-pyrrolidinol

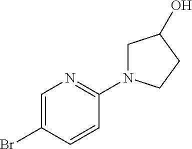

A mixture of 2,5-dibromopyridine (30.0 g, 127 mmol), pyrrolidin-3-ol hydrochloride (12.0 g, 97 mmol) in diisopropylethylamine (DIEA) (17.5 g, 136 mmol) was stirred at 140° C. for 1.5 h. After cooling to room temperature, the mixture was diluted with CH₂Cl₂ (100 mL) and washed with water (2×30 mL), brine (20 mL), dried (Na₂SO₄) and concentrated. Flash chromatography of the residue over silica gel using 35:1 CH₂Cl₂/MeOH gave the title compound as a white solid (13.3 g, 56%): ¹H NMR (400 MHz, CDCl₃) δ ppm 8.17 (s, 1H), 7.51 (dd, J=8.80, 2.40 Hz, 1H), 6.29 (d, J=8.80 Hz, 1H), 4.64 (s, 1H), 3.49-3.67 (m, 4H), 2.11-2.23 (m, 2H), 1.45-1.65 (m, 1H).

### Intermediate 2: 1-(5-bromo-2-pyridinyl)-3-pyrrolidinyl methanesulfonate

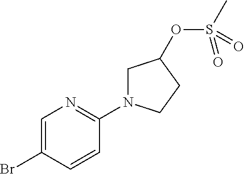

To the mixture of 1-(5-bromo-2-pyridinyl)-3-pyrrolidinol (3 g, 12.3 mmol), Et₃N (1.74 g, 17.2 mmol) in CH₂Cl₂ (50 mL) was added MsCl (1.7 g, 14.8 mmol) dropwise at 0° C. After addition, stirring was continued for 1.5 h at which time TLC analysis showed completion of the reaction. The solvent was evaporated, and the crude residue was partitioned between CH₂Cl₂ and water. The combined organic layers were washed with brine, dried (Na₂SO₄) and concentrated under reduced pressure to give crude product as a gray solid (3.6 g, 92%): ¹H NMR (400 MHz, CDCl₃) δ ppm 8.17 (d, J=2.80 Hz, 1H), 7.53 (dd, J=11.60, 3.20 Hz, 1H), 6.29 (d, J=12.00 Hz, 1H), 5.39-5.47 (m, 1H), 3.80-3.90 (m, 1H), 3.69-3.73 (m, 5.60 Hz, 1H), 3.52-3.65 (m, 2H), 3.05 (s, 3H), 2.25-2.51 (m, 2H).

### Intermediate 3: 1-(5-bromo-2-pyridinyl)-N,N-dimethyl-3-pyrrolidinamine

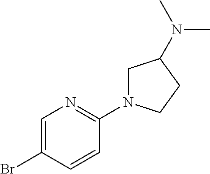

To a mixture of 1-(5-bromo-2-pyridinyl)-3-pyrrolidinyl methanesulfonate (15.0 g, 46.7 mmol), dimethylamine (33 wt %, 31.5 g, 233 mmol) in MeOH/H₂O (1:1, 20 mL) was added DIEA (15 mL, 84.4 mmol). After addition, the reaction vessel was sealed and heated at 120° C. for 15 h. The solvent was removed under reduced pressure, and the residue was purified by flash chromatography to give the title compound (2.7 g, 21%): ¹H NMR (400 MHz, CDCl₃) δ ppm 8.14 (d, J=2.40 Hz, 1H), 7.46 (dd, J=9.20, 2.40 Hz, 1H), 6.22 (d, J=9.20 Hz, 1H), 3.71 (t, J=8.40 Hz, 1H), 3.57 (t, J=8.80 Hz, 1H), 3.40-3.49 (m, 1H), 3.19 (t, J=8.80 Hz, 1H), 2.75-2.81 (m, 1H), 2.30 (s, 6H), 2.20-2.25 (m, 1H), 1.88-1.93 (m, 1H).

### Intermediate 4: 1-(5-bromo-2-pyridinyl)-N-methyl-3-pyrrolidinamine

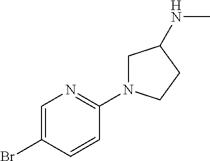

The above general procedure for Intermediate 3 was followed using 1-(5-bromo-2-pyridinyl)-3-pyrrolidinyl methanesulfonate (3.0 g, 9.34 mmol), aqueous MeNH₂ (20 wt %. 10 mL, excess), DIEA (10 mL, 58 mmol) in MeOH/H₂O (1:1, 20 mL). The title compound was obtained as a yellow solid (1.25 g, 52%): ¹H NMR (400 MHz, MeOH-d₄) δ ppm 8.14 (d, J=2.40 Hz, 1H), 7.68 (dd, J=8.80, 2.40 Hz, 1H), 6.56 (d, J=8.8 Hz, 1H), 3.89-3.96 (m, 1H), 3.75-3.85 (m, 1H), 3.61-3.71 (m, 1H), 3.49-3.59 (m, 1H), 3.40-3.47 (m, 1H), 2.95 (s, 3H), 2.38-2.58 (m, 1H), 2.05-2.30 (m, 1H).

### Intermediate 5: 1-(5-bromo-2-pyridinyl)-N-ethyl-3-pyrrolidinamine

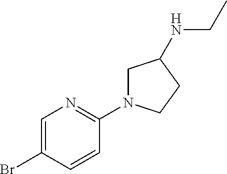

The above general procedure for Intermediate 3 was followed using 1-(5-bromo-2-pyridinyl)-3-pyrrolidinyl methanesulfonate (3.0 g, 9.34 mmol), aqueous EtNH₂ (20 wt %, 10 mL, excess), and DIEA (10 mL, 58 mmol) in MeOH/H₂O (1:1, 20 mL). The product was obtained as a yellow solid (560 mg, 22%): ¹H NMR (400 MHz, MeOH-d₄) δ ppm 7.98 (d, J=0.80 Hz, 1H), 7.45-7.55 (m, 1H), 6.32-6.40 (m, 1H), 3.55-3.63 (m, 1H), 3.45-3.55 (m, 1H), 3.22-3.44 (m, 2H), 3.15 (t, J=5.60 Hz, 1H), 2.62-2.68 (m, 2H), 2.22 (t, J=5.20 Hz, 1H), 1.84 (t, J=6.00 Hz, 1H), 1.14 (t, J=7.60 Hz, 3H).

### Intermediate 6: 1-(5-bromo-2-pyridinyl)-3-pyrrolidinamine

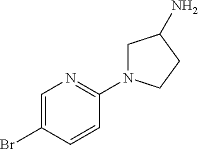

The above general procedure for Intermediate 3 was followed using 1-(5-bromo-2-pyridinyl)-3-pyrrolidinyl methanesulfonate (3.0 g, 9.34 mmol), aqueous NH₃ (15 mL, excess), DIEA (5 mL, 30 mmol). The product was obtained as a pale yellow solid (910 mg, 40%): ¹H NMR (400 MHz, DMSO-d₆) δ ppm 8.16 (d, J=2.40 Hz, 1H), 7.93 (bs, 2H), 7.68 (dd, J=9.00, 2.40 Hz, 1H), 6.49 (d, J=8.8 Hz, 1H), 3.80-3.90 (m, 1H), 3.56-3.64 (m, 1H), 3.37-3.52 (m, 3H), 2.16-2.30 (m, 1H), 1.88-2.10 (m, 1H).

### Intermediate 7: 1′-(5-bromo-2-pyridinyl)-1,3′-bipyrrolidine

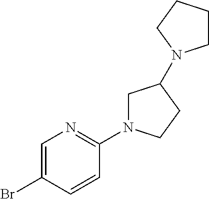

A mixture of 1-(5-bromo-2-pyridinyl)-3-pyrrolidinyl methanesulfonate (1.0 g, 3.10 mmol) in pyrrolidine (15 ml, 182 mmol) was heated at 120° C. for 18 h. The solvent was removed under reduced pressure, and the residue was purified by column chromatography to give the title compound as an orange solid (800 mg, 87%): ¹H NMR (400 MHz, MeOH-d₄) δ ppm 7.94 (d, J=1.60 Hz, 1H), 7.48 (dd, J=9.20, 2.40 Hz, 1H), 6.35 (d, J=8.80 Hz, 1H), 3.59 (t, J=7.20 Hz, 1H), 3.35-3.52 (m, 3H), 3.22-3.32 (m, 1H), 3.11-3.20 (m, 1H), 3.06-3.14 (m, 1H), 2.82-2.87 (m, 2H), 2.10-2.22 (m, 1H), 1.75-1.95 (m, 5H).

### Intermediate 8: 4-[1-(5-bromo-2-pyridinyl)-3-pyrrolidinyl]morpholine

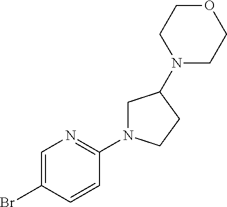

A mixture of 1-(5-bromo-2-pyridinyl)-3-pyrrolidinyl methanesulfonate (0.6 g, 1.86 mmol) in morpholine (5 mL) was heated at 60° C. for 16 h. The solvent was removed under reduced pressure, and the residue was purified by column chromatography to give the title compound as a yellow solid (500 mg, 86%): ¹H NMR (400 MHz, CDCl₃) 3 ppm 8.09 (d, J=2.00 Hz, 1H), 7.42 (dd, J=8.80, 2.40 Hz, 1H), 6.17 (d, J=8.80 Hz, 1H), 3.55-3.67 (m, 4H), 3.51-3.58 (m, 1H), 3.50-3.54 (m, 1H), 3.25-3.35 (m, 1H), 3.10-3.20 (m, 1H), 2.81-2.91 (m, 1H), 2.40-2.55 (m, 4H), 2.12-2.19 (m, 1H), 1.81-1.92 (m, 1H).

### Intermediate 9: (3S)-1-(5-bromo-2-pyridinyl)-N,N-dimethyl-3-pyrrolidinamine

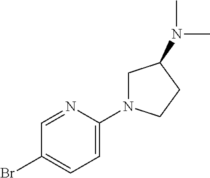

In a microwave reaction vial containing a stirring bar, a mixture of 5-bromo-2-iodopyridine (284 mg, 1.0 mmol) and (3S)—N,N-dimethyl-3-pyrrolidinamine (137 mg, 1.2 mmol) in DMF (1.0 mL) was heated to 200° C. in a microwave reactor (Emrys Optimizer from Personal Chemistry) for 30 minutes. After cooling to room temperature, the solvent was removed by evaporation under reduced pressure. The residue was loaded onto Isco for purification, eluting with dichloromethane:methanol. The title compound was obtained as a pale yellow solid (214 mg, 79%): ¹H NMR (400 MHz, MeOH-d₄) δ ppm 8.02 (d, J=2.32 Hz, 1H) 7.54 (dd, J=9.03, 2.44 Hz, 1H) 6.39 (d, J=9.03 Hz, 1H) 3.67 (dd, J=10.01, 7.32 Hz, 1H) 3.52-3.60 (m, 1H) 3.24-3.37 (m, 1H) 3.11-3.19 (m, 1H) 2.77-2.90 (m, 1H) 2.30 (s, 6H) 2.24 (ddd, J=11.99, 6.62, 5.31 Hz, 1H) 1.79-1.93 (m, 1H); ES-LCMS m/z 270, 272 (M+H)⁺.

### Intermediate 10: N-[(3R)-1-(phenylmethyl)-3-pyrrolidinyl]acetamide

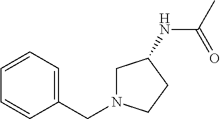

A 2L jacketed laboratory reactor was charged with a solution of (3R)-1-(phenylmethyl)-3-pyrrolidinamine (137 g, 0.777 mol) in dichloromethane (DCM, 1 L), the jacket temperature was set to 20° C., and neat acetic anhydride (75 mL, 0.795 mol) was added slowly dropwise while maintaining a gentle reflux; the addition required ca. 15 min. The reaction mixture was stirred for about 1 hr, and allowed to return to 20° C. The solution was washed three times with 5% Na₂CO₃ solution (3×1 L); the layers were separated and the DCM layer was set aside. The combined aqueous layers were extracted once with DCM (350 mL), and the combined DCM layers were dried over MgSO₄, filtered and concentrated by rotovap and then under high vacuum, to afford a light amber oil (157.45 g, 93%): ¹H NMR (400 MHz, CDCl₃) δ ppm 1.48-1.63 (m, 1H), 1.91 (s, 3H), 2.16-2.32 (m, 2H), 2.46-2.59 (m, 2H), 2.79-2.87 (m, 1H), 3.57 (s, 2H), 4.35-4.46 (m, 1H), 5.78-5.92 (m, 1H), 7.19-7.34 (m, 5H).

### Intermediate 11: (3R)—N-ethyl-1-(phenylmethyl)-3-pyrrolidinamine

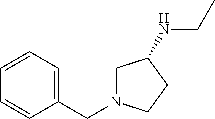

To a mechanically stirred solution of N-[(3R)-1-(phenylmethyl)-3-pyrrolidinyl]acetamide (157.4 g, 721 mmol) in tetrahydrofuran (THF) (300 ml) at ambient temperature was added lithium aluminum hydride (1.3 L, 1.3 mol, 1M in THF) dropwise over 1.5 hrs. The reaction was heated at reflux for 6 hrs, and then allowed to stir at ambient temperature overnight. The reaction was cooled to 5° C. and quenched by the very slow addition of water (80 mL) followed by 15% NaOH (80 mL) and additional water (240 mL). This mixture was allowed to stir for 1 hr before filtering. The filter cake was rinsed with THF (2×400 mL), and the filtrate was concentrated. Fresh THF (500 mL) was added, and the mixture was concentrated again to afford the desired crude product as a yellow oil (140.1 g, 95%): ¹H NMR (400 MHz, CDCl₃) δ ppm 7.18-7.32 (m, 5H), 3.57 (d, J=1.64 Hz, 2H), 3.24-3.33 (m, 1H), 2.72 (dd, J=9.25, 6.78 Hz, 1H), 2.45-2.63 (m, 3H), 2.31 (dd, J=9.35, 5.14 Hz, 1H), 2.05-2.16 (m, 1H), 1.53 (dddd, J=13.06, 7.93, 5.40, 5.27 Hz, 1H), 1.06 (t, J=7.14 Hz, 3H).

### Intermediate 12: (3R)—N-ethyl-3-pyrrolidinamine

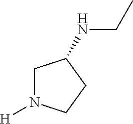

To a mechanically stirred solution of (3R)—N-ethyl-1-(phenylmethyl)-3-pyrrolidinamine (140 g, 686 mmol) in methanol (1.2 L) was added palladium hydroxide on carbon (18 g, 25.6 mmol) and ammonium formate (173 g, 2743 mmol). The reaction was heated at reflux for 2.5 hrs. After cooling to RT, the reaction was filtered and the filtrate was concentrated. The oil was taken up in THF (800 mL) and cooled in an ice bath. 50% aqueous sodium hydroxide (71 mL) was added, and the mixture was stirred for 15 minutes. Magnesium sulfate (35 g) was added, and the mixture was stirred for 15 minutes. The mixture was diluted with DCM (1 L), and filtered through Celite. The filtrate was concentrated, taken up in fresh DCM, dried over magnesium sulfate, and concentrated to afford the desired crude product as a yellow oil (47.8 g, 61%): ¹H NMR (400 MHz, CDCl₃) δ ppm 3.19-3.28 (m, 1H), 3.04 (ddd, J=10.97, 7.99, 6.11 Hz, 1H), 2.95 (dd, J=11.41, 6.06 Hz, 1H), 2.80-2.89 (m, 1H), 2.72 (dd, J=11.36, 4.06 Hz, 1H), 2.52-2.65 (m, 3H), 1.95 (td, J=13.36, 7.61 Hz, 1H), 1.43-1.54 (m, 1H), 1.07 (t, J=7.09 Hz, 3H).

### Intermediate 13: (3R)-1-(5-bromo-2-pyridinyl)-N-ethyl-3-pyrrolidinamine

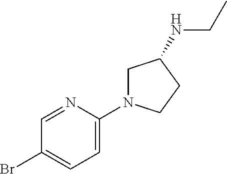

The 5-bromo-2-fluoropyridine (1.541 g, 8.76 mmol) in CH₃CN (3 mL) was treated with DIEA (1.530 mL, 8.76 mmol) followed by (3R)—N-ethyl-3-pyrrolidinamine (1 g, 8.76 mmol). The reaction was stirred at room temperature for 15 hours whereupon LCMS indicated 90% product. The reaction was diluted with EtOAc and 1N NaOH. The layers were separated, and the aqueous layer was extracted with EtOAc. The combined organic layers were washed with brine, dried (Na₂SO₄), filtered, and concentrated in vacuo to give 1.3 g of the title compound as an orange oil: ¹H NMR (400 MHz, CDCl₃) δ ppm 8.11 (dd, J=2.9, 0.8 Hz, 1H), 7.43 (dd, J=8.8, 2.6 Hz, 1H), 6.20 (dd, J=8.8, 0.8 Hz, 1H), 3.62 (dd, J=10.3, 6.2 Hz, 1H), 3.55-3.41 (m, 2H), 3.37 (dt, J=9.9, 7.3 Hz, 1H), 3.16 (dd, J=10.1, 5.2, 1H), 2.67 (q, J=7.1 Hz, 2H), 2.24-2.13 (m, 1H), 1.88-1.77 (m, 1H), 1.1 (t, J=7.1, 3H); ES-LCMS m/z 270, 272 (M+H)⁺.

An alternative procedure was use for a larger-scale synthesis of the title compound. Thus, a mixture of (3R)—N-ethyl-3-pyrrolidinamine (47.8 g, 419 mmol), 5-bromo-2-fluoropyridine (70.0 g, 398 mmol), DIEA (88 ml, 502 mmol), and acetonitrile (50 mL) was stirred at ambient temperature for 16 hrs. HPLC showed that the reaction was ˜60% complete. The reaction was warmed at 60° C. for 18 hrs, then returned to ambient temperature. The mixture was diluted with ethyl acetate (1 L), and saturated aqueous sodium bicarbonate (1 L) was added. The layers were separated, and the aqueous layer was extracted with ethyl acetate (600 mL). The combined ethyl acetate was dried over magnesium sulfate and concentrated. The residue was purified by silica gel chromatography (95:5/chloroform:methanol, followed by 90:10:2/chloroform:methanol:ammonium hydroxide), to afford the desired product as a pale yellow oil that crystallized on standing (50.6 g, 47%): ¹H NMR (400 MHz, CDCl₃) δ ppm 8.11 (d, J=2.36 Hz, 1H), 7.43 (dd, J=8.94, 2.36 Hz, 1H), 6.20 (d, J=8.94 Hz, 1H), 3.62 (dd, J=10.28, 6.27 Hz, 1H), 3.41-3.56 (m, 2H), 3.36 (dt, J=9.92, 7.32 Hz, 1H), 3.17 (dd, J=10.28, 5.34 Hz, 1H), 2.68 (q, J=7.19 Hz, 2H), 2.12-2.24 (m, 1H), 1.77-1.88 (m, 1H), 1.10 (t, J=7.09 Hz, 3H). Fractions containing ˜2-3% of the starting pyridine were combined and concentrated to afford additional product (11.2 g, 10%).

### Intermediate 14: (3S)-1-(5-bromo-2-pyridinyl)-N-ethyl-3-pyrrolidinamine

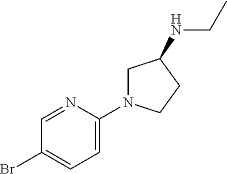

The 5-bromo-2-fluoropyridine (1.541 g, 8.76 mmol) in CH₃CN (3 mL) was treated with DIEA (1.530 mL, 8.76 mmol) followed by (3S)—N-ethyl-3-pyrrolidinamine (1 g, 8.76 mmol). The reaction was stirred at room temperature for 15 hours whereupon LCMS indicated 90% product. The reaction was diluted with EtOAc and 1N NaOH. The layers were separated, and the aqueous layer was extracted with EtOAc. The combined organic layers were washed with brine, dried (Na₂SO₄), filtered, and concentrated in vacuo to give the title compound as an orange oil (1.3 g, 55%): ¹H NMR (400 MHz, CDCl₃) δ ppm 8.11 (dd, J=2.9, 0.8 Hz, 1H), 7.43 (dd, J=8.8, 2.6 Hz, 1H), 6.20 (dd, J=8.8, 0.8 Hz, 1H), 3.62 (dd, J=10.3, 6.2 Hz, 1H), 3.55-3.41 (m, 2H), 3.37 (dt, J=9.9, 7.3 Hz, 1H), 3.16 (dd, J=10.1, 5.2, 1H), 2.67 (q, J=7.1 Hz, 2H), 2.24-2.13 (m, 1H), 1.88-1.77 (m, 1H), 1.1 (t, J=7.1, 3H); ES-LCMS m/z 270, 272 (M+H)⁺.

### Intermediate 15: methyl 5-chloro-2-pyridinecarboxylate

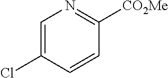

To a solution of 2-bromo-5-chloropyridine (30.0 g, 155.9 mmol) in MeOH (280 mL) was added Pd(OAc)₂ (3.5 g, 10.8 mmol), dppf (17.3 g, 37.96 mmol), Et₃N (42.0 mL, 312 mmol). The resulting mixture was stirred at 50° C. under a CO atmosphere (15 psi) for 24 h, then concentrated under reduced pressure to give crude residue. This residue was partitioned between EtOAc (3×500 mL) and water (300 mL). The combined organic layers were dried (Na₂SO₄) and evaporated. Flash chromatography of the residue over silica gel, by using 10:1 petroleum ether/EtOAc, afforded the title compound as a pale yellow solid (25 g, 93%): ¹H NMR (400 MHz, CDCl₃) 5 ppm 8.60 (d, J=1.60 Hz, 1H), 8.01 (d, J=8.40 Hz, 1H), 7.75 (dd, J=8.40, 2.40 Hz, 1H), 3.92 (s, 3H).

### Intermediate 16: (5-chloro-2-pyridinyl)methanol

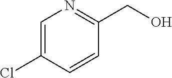

To a cooled (0° C.) solution of methyl 5-chloro-2-pyridinecarboxylate (43 g, 251 mmol) in methanol (400 mL) was added NaBH₄ (28.7 g, 754 mmol) in small portions over approximately 30 min. After addition, the reaction mixture was stirred at room temperature for 2 h, at which time TLC analysis showed the completion of the reaction. The reaction mixture was then concentrated under reduced pressure, and the residue was adjusted to pH 1 by adding 1N HCl. The resulting solution was extracted with EtOAc (3×300 mL). The combined organic layers were dried (Na₂SO₄) and evaporated. Flash chromatography of the residue over silica gel using 10:1 petroleum ether/EtOAc as eluent provided the title compound (36 g, 99%): ¹H NMR (400 MHz, CDCl₃) δ ppm 8.44 (d, J=1.60 Hz, 1H), 7.62 (dd, J=8.40, 2.40 Hz, 1H), 7.25 (d, J=8.40 Hz, 1H), 4.69 (s, 2H), 3.83 (s, 1H).

### Intermediate 17: 4-{[(5-chloro-2-pyridinyl)methyl]oxy}pyridine-1-oxide

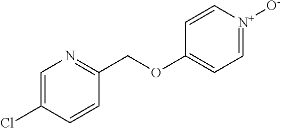

Sodium (7.5 g, 326 mmol) was added to a solution of (5-chloro-2-pyridinyl)methanol (36 g, 252 mmol) in THF (400 mL). After addition, the mixture was stirred at reflux for 16 h and then cooled to room temperature. To this mixture, a solution of 4-nitropyridine N-oxide (11.7 g, 84 mmol) in THF (100 mL) was added and the resulting mixture was stirred at room temperature for another 4 h. The mixture was filtered and the filtrate was concentrated under reduced pressure. Et₂O was added and a precipitate was formed. The precipitate was collected by filtration and washed with Et₂O (3×). This solid was dissolved in CH₂Cl₂ and filtered. The filtrate was dried (Na₂SO₄) and evaporated to give the title compound (9.7 g, 49%): ¹H NMR (400 MHz, CDCl₃) δ ppm 8.54 (d, J=0.80 Hz, 1H), 8.09 (m, 2H), 7.71 (dd, J=8.40, 2.40 Hz, 2H), 7.39 (dd, J=8.40, 0.40 Hz, 1H), 6.87 (m, 2H), 5.17 (s, 2H).

An alternative procedure was use for a larger-scale synthesis of the title compound. Thus, a stirred mixture of (5-chloro-2-pyridinyl)methanol (15.36 g, 107 mmol) and 4-nitropyridine 1-oxide (14.99 g, 107 mmol) in DCM (250 ml) cooled in an ice/water bath was charged with benzyltriethylammonium chloride (0.682 g, 3.00 mmol), and 9M NaOH (140 mL) was added dropwise via addition funnel. The mixture was stirred for 2.5 hours at room temperature with periodic checking by HPLC. The reaction mixture became a dark solution over this time period with easier stirring. LC/MS indicated that the reaction was complete. Water (300 mL) was added to the reaction and it quickly became an oily suspension. The reaction mixture was diluted with DCM and the organic layer was separated. The aqueous layer was extracted 3 more times with DCM, and the combined organic layers were washed with brine and dried over sodium sulfate. Concentration yielded a bright yellow solid, which was collected, washed with ether, and dried overnight (22.37 g, 88%): ES-LCMS m/z 237 (M+H)⁺.

### Intermediate 18: 4-{[(5-chloro-2-pyridinyl)methyl]oxy}-2(1H)-pyridinone

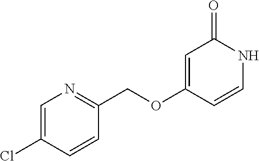

trifluoroacetic anhydride (TFAA) (9.7 g, 46.6 mmol) was added dropwise to a stirred and cooled (0° C.) solution of 4-{[(5-chloro-2-pyridinyl)methyl]oxy}pyridine-1-oxide (1.1 g, 4.7 mmol) and Et₃N (1.4 g, 14.0 mmol) in THF (15 mL). The reaction mixture was stirred at room temperature for 16 h, at which time TLC analysis showed almost completion of the reaction. The reaction mixture was diluted with water, and then extracted with CH₂Cl₂ (3 X). The combined organic layers were washed with water, 1 N NaOH, brine, dried and concentrated in vacuo. The residue solid was triturated with ether to give the title compound (850 mg, 77%): ¹HNMR (400 MHz, DMSO-d₆) δ ppm 11.11 (s, 1H), 8.61 (s, 1H), 7.96 (d, J=6.00 Hz, 1H), 7.52 (d, J=8.40 Hz, 1H), 7.23 (d, J=7.60 Hz, 1H), 5.92 (d, J=4.80 Hz, 1H), 5.73 (s, 1H), 5.10 (s, 2H); ES-LCMS m/z 237 (M+H).

An alternative procedure was use for a larger-scale synthesis of the title compound. Thus, 4-{[(5-chloro-2-pyridinyl)methyl]oxy}pyridine-1-oxide (25 g, 106 mmol) and triethylamine (44.2 mL, 317 mmol) were allowed to stir in 300 mL of THF while cooling in an ice bath. Trifluoroacetic anhydride (224 mL, 1585 mmol) was added dropwise via addition funnel. The reaction mixture was allowed to stir an additional 15 min at ice bath temperature, and then warmed to room temperature. The reaction was allowed to stir overnight at room temperature. The next morning, LC/MS indicated that the reaction was complete. The reaction was poured over ice, and the resulting solution was extracted with DCM (4×100 mL). The organic layers were combined, washed with water, 1N NaOH, saturated brine solution, dried over sodium sulfate, and concentrated. The resulting solid was purified via chromatography using a gradient of (0-100% EtOAC/hexanes over a 30 minute run), to provide the title compound as a white solid (15 g, 60%).

### Intermediate 19: methyl isonicotinate N-oxide

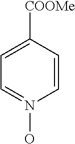

To methyl isonicotinate (13.70 g, 100 mmol) and methyltrioxorhenium (125 mg, 0.5 mmol) in dichloromethane (40 mL) was added dropwise 30% hydrogen peroxide/water (20 mL, 200 mmol), and the mixture was stirred at ambient temperature for 18 h. Manganese dioxide (40 mg) was slowly added and vigorous bubbling occurred. After 2 h stirring at ambient temperature, water/brine (1:1) was added, and the mixture was extracted with dichloromethane (3×). The organic layer was dried over sodium sulfate, and concentrated to provide the title compound as a pale yellow solid (15.2 g, 99%): ¹H NMR (400 MHz, DMSO-d₆) δ ppm 8.28 (d, J=7.1 Hz, 2H), 7.82 (d, J=7.1 Hz, 2H), 3.84 (s, 3H).

### Intermediate 20: methyl 2-(acetyloxy)-4-pyridinecarboxylate

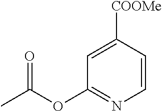

A mixture of methyl isonicotinate N-oxide (15.0 g, 97.9 mmol) and acetic anhydride (150 mL) was heated to 14° C. for 6 h. The mixture was concentrated, and the residue was heated to 60° C. with methanol and activated charcoal (Darco G-60) for 15 min, then filtered through a bed of Celite. The filtrate was concentrated, and the residue was triturated with diethyl ether. The solid was filtered to provide recovered methyl isonicotinate N-oxide (4.0 g, 26%). The filtrate was washed with saturated aqueous sodium bicarbonate, brine, and dried over sodium sulfate. The solution was concentrated and purified by column chromatography on silica gel, eluting with 2% methanol:dichloromethane, to afford the title compound as a pale yellow solid (5.0 g, 26%): ¹H NMR (400 MHz, DMSO-d₆) δ ppm 8.56 (d, J=5.0 Hz, 1H), 7.77 (d, J=5.0 Hz, 1H), 7.65 (s, 1H), 3.89 (s, 3H), 2.30 (s, 3H).

### Intermediate 21: methyl 2-oxo-1,2-dihydro-4-pyridinecarboxylate

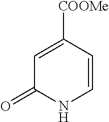

Methyl 2-(acetyloxy)-4-pyridinecarboxylate (5.0 g) and methanol (50 mL) were heated at 73° C. for 18 h, then concentrated to provide the title compound as a pale yellow solid (3.67 g, 94%): ¹H NMR (400 MHz, DMSO-d₆) δ ppm 11.9 (br s, 1H), 7.49 (d, J=6.6 Hz, 1H), 6.78 (s, 1H), 6.48 (dd, J=6.6, 1.4 Hz, 1H), 3.81 (s, 3H); ES-LCMS m/z 154 (M+H)⁺.

### Intermediate 22: 4-(hydroxymethyl)-2(1H)-pyridinone

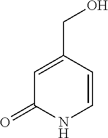

To a suspension of methyl 2-oxo-1,2-dihydro-4-pyridinecarboxylate (1.37 g, 8.98 mmol) in anhydrous tetrahydrofuran (23 mL) was added dropwise 2 M lithium borohydride/tetrahydrofuran (22.5 mL, 45 mmol), and the mixture was heated to 55° C. under a nitrogen atmosphere for 3.5 h. Methanol (15 mL) and water (3 mL) were carefully added, and the mixture was stirred at ambient temperature for 30 min. The mixture was concentrated and more methanol (10 mL) was added carefully. After stirring for 30 min, the mixture was adsorbed on silica gel and placed on top of a silica gel column, eluting with 0 to 30% methanol:dichloromethane, to obtain the title compound as an off-white solid (0.99 g, 88%): ¹H NMR (400 MHz, DMSO-d₆) δ ppm 11.29 (br s, 1H), 7.23 (d, J=6.7 Hz, 1H), 6.21 (s, 1H), 6.03 (dd, J=6.7, 1.3 Hz, 1H), 5.27 (t, J=5.9 Hz, 1H), 4.28 (d, J=5.9 Hz, 2H).

### Intermediate 23: 4-({[(1,1-dimethylethyl)(dimethyl)silyl]oxy}methyl)-2(1H)-pyridinone

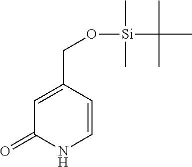

To a suspension of 4-(hydroxymethyl)-2(1H)-pyridinone (0.98 g, 7.9 mmol) and DMF (10 mL) was added imidazole (0.64 g, 9.45 mmol) and tert-butyldimethylsilyl chloride (1.25 g, 8.26 mmol), and the mixture was stirred at ambient temperature under a nitrogen atmosphere for 18 h. The mixture was poured into water (30 mL), and stirred for 30 min. The solid was filtered, rinsed with water, and air-dried to provide 4-({[(1,1-dimethylethyl)(dimethyl)silyl]oxy}methyl)-2(1H)-pyridinone as an off-white solid (1.64 g, 88%): ¹H NMR (400 MHz, DMSO-d₆) δ ppm 11.34 (br s, 1H), 7.26 (d, J=6.6 Hz, 1H), 6.21 (s, 1H), 6.01 (dd, J=6.6, 1.5 Hz, 1H), 4.5 (s, 2H), 0.88 (s, 9H), 0.05 (s, 6H); EI-LCMS m/z 241 (M+H)⁺.

### Intermediate 24: 6′-[3-(dimethylamino)-1-pyrrolidinyl]-4-({[(1,1-dimethylethyl)(dimethyl)silyl]oxy}methyl)-2H-1,3′-bipyridin-2-one

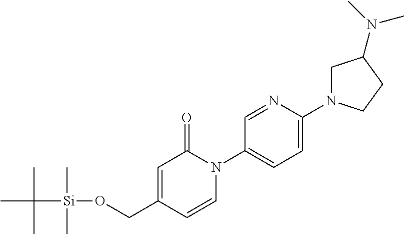

A mixture of 4-({[(1,1-dimethylethyl)(dimethyl)silyl]oxy}methyl)-2(1H)-pyridinone (0.1 g, 0.41 mmol), 1-(5-bromo-2-pyridinyl)-N,N-dimethyl-3-pyrrolidinamine (0.11 g, 0.41 mmol), copper (I) iodide (39 mg, 0.21 mmol), trans-N,N′-dimethyl-1,2-cyclohexanediamine (29 mg, 0.41 mmol), potassium carbonate (0.113 g, 0.82 mmol), and anhydrous 1,4-dioxane (3.5 mL) was degassed with a stream of nitrogen for 5 min, sealed, and heated to 120° C. for 18 h. The mixture was diluted with ethyl acetate, filtered through a bed of Celite, and the filtrate was washed with dilute (5%) aqueous ammonium hydroxide (2×), brine, dried over sodium sulfate, and concentrated. The residue was purified by chromatography on silica gel, eluting with 2 M methanolic ammonia in dichloromethane 1:19, to afford the title compound as an off-white solid (0.124 g, 69%): ¹H NMR (400 MHz, DMSO-d₆) δ ppm 8.0 (d, J=2.6 Hz, 1H), 7.53 (d, J=7.0 Hz, 1H), 7.5 (dd, J=9.0, 2.6 Hz, 1H), 6.5 (d, J=9.0 Hz, 1H), 6.35 (d, J=1.4 Hz, 1H), 6.15 (dd, J=7.0, 1.4 Hz, 1H), 4.57 (s, 2H), 3.68 (dd, J=10.0, 7.1 Hz, 1H), 3.58 (t, J=8.4 Hz, 1H), 3.36-3.33 (m, 1H), 3.12 (dd, J=10.0, 8.4 Hz, 1H), 2.80-2.74 (m, 1H), 2.19 (s, 6H), 2.18-2.14 (m, 1H), 1.84-1.74 (m, 1H), 0.89 (s, 9H), 0.08 (s, 6H); EI-LCMS m/z 429 (M+H)⁺.

### Intermediate 25: 6′-[3-(dimethylamino)-1-pyrrolidinyl]-4-(hydroxymethyl)-2H-1,3′-bipyridin-2-one

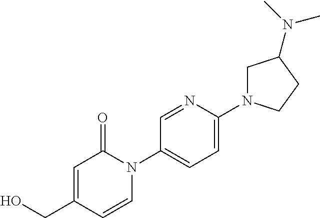

Cold trifluoroacetic acid/water (9:1, 2 mL) was added to 6′-[3-(dimethylamino)-1-pyrrolidinyl]-4-({[(1,1-dimethylethyl)(dimethyl)silyl]oxy}methyl)-2H-1,3-bipyridin-2-one (0.12 g), and the mixture was stirred at 0° C. for 3 h. The mixture was concentrated, and the residue was partitioned between dichloromethane and a small amount of saturated aqueous sodium bicarbonate. The aqueous phase was extracted with dichloromethane (3×) and the combined organic extracts were dried over sodium sulfate and concentrated to provide the title compound as a glass (72 mg, 79%): ¹H NMR (400 MHz, DMSO-d₆) δ ppm 7.98 (d, J=2.4 Hz, 1H), 7.49-7.47 (m, 2H), 6.49 (d, J=9.1 Hz, 1H), 6.34 (s, 1H), 6.16 (br d, J=7.0 Hz, 1H), 5.36 (t, J=5.9 Hz, 1H), 4.33 (d, J=5.9 Hz, 2H), 3.67 (dd, J=10.0, 7.0 Hz, 1H), 3.57 (t, J=9.1 Hz, 1H), 3.35-3.30 (m, 1H), 3.15-3.10 (m, 1H), 2.9-2.7 (m, 1H), 2.3-2.1 (m, 7H), 1.9-1.7 (m, 1H); EI-LCMS m/z 315 (M+H)⁺.

### Intermediate 26: 6′-[3-(dimethylamino)-1-pyrrolidinyl]-4-[(phenylmethyl)oxy]-2H-1,3′-bipyridin-2-one

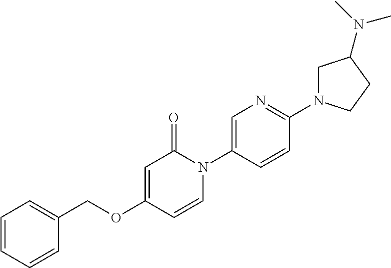

Commercially available 4-[(phenylmethyl)oxy]-2(1H)-pyridinone (2 g, 9.94 mmol), 1-(5-bromo-2-pyridinyl)-N,N-dimethyl-3-pyrrolidinamine (2.69 g, 9.94 mmol), K₂CO₃ (2.75 g, 19.88 mmol), and CuI (0.379 g, 1.988 mmol) were combined, followed by a solution of trans-N,N′-dimethyl-1,2-cyclohexanediamine (0.283 g, 1.988 mmol) in toluene (12 mL). The reaction mixture was stirred in a sealed tube at 160° C. for 24 h, then cooled to 25° C. and diluted with dichloromethane/methanol. The reaction mixture was filtered through Celite, and the filtrate was concentrated in vacuo to give a solid. Recrystallization from ethyl acetate provided the title compound as a gray solid (3.5 g, 90%): ¹H NMR (400 MHz, MeOH-d₄) δ ppm 7.96 (d, J=2.6 Hz, 1H), 7.52-7.28 (m, 7H), 6.56 (d, J=9.0 Hz, 1H), 6.22 (dd, J=7.7, 2.8 Hz, 1H), 6.05 (d, J=2.8 Hz, 1H), 5.12 (s, 2H), 3.78 (dd, J=10.2, 7.3 Hz, 1H), 3.66 (t, J=8.8 Hz, 1H), 3.42 (dt, J=10.2, 6.9 Hz, 1H), 3.30-3.20 (m, 1H), 2.98-2.85 (m, 1H), 2.37-2.23 (m, 1H), 1.99-1.83 (m, 1H); ES-LCMS m/z 391 (M+H)⁺.

### Intermediate 27: 6′-[3-(dimethylamino)-1-pyrrolidinyl]-4-hydroxy-2H-1,3′-bipyridin-2-one

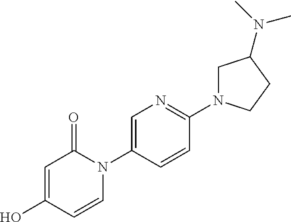

A solution of 6′-[3-(dimethylamino)-1-pyrrolidinyl]-4-[(phenylmethyl)oxy]-2H-1,3′-bipyridin-2-one (3.5 g, 8.96 mmol) in MeOH was treated with 10% palladium on carbon (0.286 g, 0.269 mmol), then stirred under a hydrogen balloon for 16 h. The reaction mixture was filtered through Celite, and the filtrate was concentrated in vacuo to provide the title compound as a beige solid (2.35 g, 87%): ¹H NMR (400 MHz, MeOH-d₄) δ ppm 7.96 (d, J=2.4 Hz, 1H), 7.48 (dd, J=8.8, 2.6 Hz, 1H), 7.39 (d, J=7.5 Hz, 1H), 6.56 (d, J=9.0, 1H), 6.09 (d, J=7.5 Hz, 1H), 3.79 (dd, J=10.3, 7.3 Hz, 1H), 3.67 (br t, J=10.5 Hz, 1H), 3.42 (dt, J=10.2, 7.1 Hz, 1H), 3.32-3.24 (m, 1H), 3.08-2.96 (m, 1H), 2.38 (s, 6H), 2.36-2.26 (m, 1H), 2.00-1.87 (m, 1 H); ES-LCMS m/z 301 (M+H)⁺.

### Intermediate 28: 1-(5-bromo-3-methyl-2-pyridinyl)-N,N-dimethyl-3-pyrrolidinamine

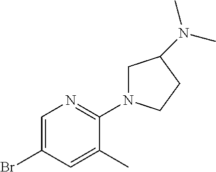

To a 250 mL round-bottomed flask was charged 5-bromo-2-fluoro-3-methylpyridine (3 g, 15.79 mmol), N,N-dimethylpyrrolidine (4.51 g, 39.5 mmol) in acetonitrile (125 mL). The reaction mixture was stirred at room temperature for 18 hours. The reaction was diluted with 250 mL of ethyl acetate, and then washed with 1N sodium carbonate aqueous (2×). The aqueous layer was extracted twice with ethyl acetate, and the combined organic layers were then dried over anhydrous sodium sulfate, and concentrated. The crude product precipitated during concentration, and was washed with 5% methanol in dichloromethane, to give the title compound (1.5 g, 30%): ¹H NMR (400 MHz, CDCl₃) δ ppm 8.02 (s, 1H), 7.37 (s, 1H), 3.62-3.49 (m, 4H) 2.74 (t, J=4.3 Hz, 1H), 2.27 (s, 6H), 2.15 (s, 3H), 2.06 (m, 1H), 1.86 (m, 1H).

### Intermediate 29: 1-(5-bromo-2-pyridinyl)-N-methyl-N-(1-methylethyl)-3-pyrrolidinamine

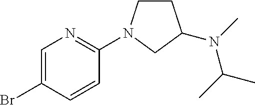

1-(5-bromo-2-pyridinyl)-3-pyrrolidinyl methanesulfonate (200 mg, 0.623 mmol) was dissolved in anhydrous acetonitrile (5 mL, 0.125M) and treated with excess N-methyl-2-propanamine (3 ml), then the reaction vessel was sealed. The reaction was heated to 100° C., and allowed to stir for 15 h. After cooling to 25° C., the solvent was removed under reduced pressure, and the residue purified by flash chromatography to give the title compound (127 mg, 68%): ¹H NMR (400 MHz, MeOH-d₄) δ ppm 8.03 (d, J=0.64 Hz, 1H), 7.55 (dd, J=9.03, 2.58 Hz, 1H), 6.43 (dd, J=9.03 Hz, 0.64 Hz, 1H), 3.65-3.76 (m, 2H), 3.45-3.61 (m, 2H), 3.03-3.17 (m, 3H), 2.18-2.41 (m, 3H), 1.81-1.93 (m, 1H), 1.03-1.08 (d, 6H).

### Intermediate 30:1-(5-bromo-2-pyridinyl)-N-ethyl-N-methyl-3-pyrrolidinamine

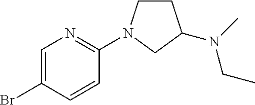

1-(5-bromo-2-pyridinyl)-3-pyrrolidinyl methanesulfonate (200 mg, 0.623 mmol) was dissolved in anhydrous acetonitrile (5 mL, 0.125M), then treated with excess N-methylethanamine (3 mL). The reaction vessel was sealed, then heated to 100° C. and stirred for 15 h. After cooling to 25° C., the solvent was removed under reduced pressure, and the residue was purified by flash chromatography to give the title compound (162 mg, 88%): ¹H NMR (400 MHz, CDCl₃) δ ppm 8.15 (s, 1H), 7.48 (dd, J=8.96, 2.50 Hz, 1H), 6.24 (d, J=8.96 Hz, 1H), 3.71-3.76 (m, 2H), 3.60-3.75 (m, 2H), 3.32-3.37 (m, 2H), 2.55 (br. s., 2H), 2.30 (s, 3H), 2.23-2.28 (m, 1H), 1.08-1.11 (d, 3H).

### Intermediate 31: 1-(5-bromo-2-pyridinyl)-N-cyclohexyl-3-pyrrolidinamine

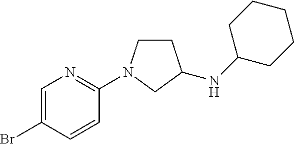

A mixture of 1-(5-bromo-2-pyridinyl)-N-cyclohexyl-3-pyrrolidinamine (500 mg, 1.557 mmol) and cyclohexylamine (154 mg, 1.557 mmol) in a sealed reaction vessel was heated to 120° C., and allowed to stir overnight. The crude reaction mixture was concentrated in vacuo, then purified by flash chromatography to give the title compound (195 mg, 39%): ¹H NMR (400 MHz, DMSO-d₆) δ ppm 8.07 (dd, J=2.28, 0.42 Hz, 1H), 7.58 (dd, J=8.98, 2.57 Hz, 1H), 6.37-6.39 (m, 1H), 3.38-3.54 (m, 2H), 3.23-3.34 (m, 3H), 3.13-3.16 (m, 1H), 2.97-3.04 (m, 1H), 2.37-2.45 (m, 1H) 2.02-2.11 (m, 1H) 21.46-1.87 (m, 6H), 0.90-1.27 (m, 5H); ES-LCMS m/z 325 (M+H)⁺.

### Intermediate 32: 4-{[(5-chloro-2-pyridinyl)methyl]oxy}-6′-(amino)-2H-1,3′-bipyridin-2-one

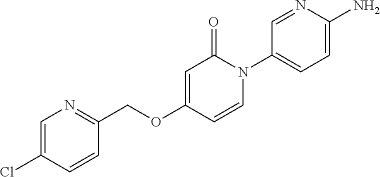

To a solution of 4-{[(5-chloro-2-pyridinyl)methyl]oxy}-2(1H)-pyridinone 9 g, 38 mmol) in anhydrous DMF (250 mL) was added 2-amino-5-iodo pyridine (9.18 g, 41.7 mmol), CuI (1.5 g, 7.56 mmol), K₂CO₃ (15.7 g, 114 mmol), and 8-hydroxyquinoline (0.9 g, 7.2 mmol), and the mixture was heated at 120° C. for 12 h. After LC-MS showed the stating material was consumed, the solvent was removed in vacuo to give the crude product, which was purified by column chromatography (EA/PE=3:1, to EA to DCM/MeOH=10:1, to MeOH) to afford 4-{[(5-chloro-2-pyridinyl)methyl]oxy}-6′-(amino)-2H-1,3′-bipyridin-2-one (8.0 g, 71.9%): ¹H NMR (400 MHz, MeOH-d₄) δ ppm 8.51 (d, J=1.60 Hz, 1H), 7.83-7.87 (m, 2H), 7.52 (d, J=8.40 Hz, 1H), 7.45 (d, J=7.60 Hz, 1H), 7.39 (d, J=8.80 Hz, 1H), 6.61 (t, J=8.00 Hz, 1H), 6.24 (t, J=8.00 Hz, 1H), 6.00 (d, J=2.80 Hz, 1H), 5.17 (s, 2H).

### Intermediate 33: 4-{[(5-chloro-2-pyridinyl)methyl]oxy}-6′-(fluoro)-2H-1,3′-bipyridin-2-one

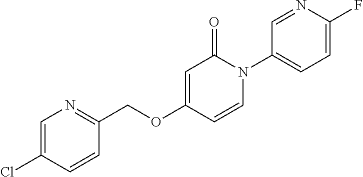

To a solution of HF/pyridine (50 mL) in pyridine (50 mL) in an ice bath was added 4-{[(5-chloro-2-pyridinyl)methyl]oxy}-6′-(amino)-2H-1,3′-bipyridin-2-one (5.0 g, 15.2 mmol). After stirring at room temperature for 30 min, the mixture was cooled at −20° C. NaNO₂ (1.5 g, 20 mmol) was added, and the reaction mixture was stirred at room temperature for 2 h. After TLC showed the starting material was completely consumed, the mixture was poured into saturated aqueous K₂CO₃ solution (200 mL) at 0° C. with stirring. The mixture was extracted with EA (3×800 mL), and the combined organic layer was dried over MgSO₄, and concentrated to give 4-{[(5-chloro-2-pyridinyl)methyl]oxy}-6′-(fluoro)-2H-1,3′-bipyridin-2-one (4.7 g, 93%): ¹H NMR (400 MHz, MeOH-d₄) δ ppm 8.57 (d, J=2.40 Hz, 1H), 8.24 (d, J=2.00 Hz, 1H), 8.03 (d, J=8.00 Hz, 1H), 7.91 (dd, J=7.60, 2.40 Hz, 1H), 7.58 (d, J=7.60 Hz, 2H), 7.20 (dd, J=7.20, 2.80 Hz, 1H), 6.32 (dd, J=7.60, 2.40 Hz, 1H), 6.08 (d, J=2.40 Hz, 1H), 5.23 (s, 2H).

### Intermediate 34: methyl 1-benzyl-4-methylpyrrolidine-3-carboxylate

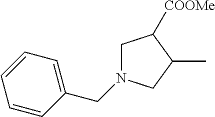

To a solution of (E)-methyl but-2-enoate (23 g, 229.73 mmol) in dry CH₂Cl₂ (containing 0.5% TFA, 700 mL) was added N-benzyl-1-methoxy-N-[(trimethylsilyl)methyl]methanamine (68.18 g, 221.73 mmol) at 0° C. under a nitrogen atmosphere, and the mixture was stirred overnight at room temperature. The reaction mixture was washed with saturated aqueous Na₂CO₃ (100 mL), dried over Na₂SO₄, concentrated, and distilled to give methyl 1-benzyl-4-methylpyrrolidine-3-carboxylate (40 g, 74.6%, 110° C., 5 mm Hg) as a colorless oil: ¹H NMR (400 MHz, CDCl₃) δ ppm 1.1 (s, 3H), 2.2 (t, 1H), 2.4-2.6 (t, 2H), 2.7-2.9 (t, 1H), 3.6-3.8 (t, 5H), 7.2-7.4 (t, 5H); LCMS m/z 234 (M+H)⁺.

### Intermediate 35: 1-benzyl-3-(tert-butyloxycarbonylamino)-4-methylpyrrolidine

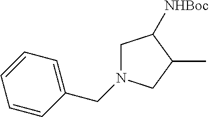

A solution of compound methyl 1-benzyl-4-methylpyrrolidine-3-carboxylate (40 g, 171 mmol) in aqueous HCl (12M, 300 mL) was heated at 70-80° C. overnight. The reaction solution was concentrated in high vacuo to give crude 1-benzyl-4-methylpyrrolidine-3-carboxylic acid (45 g, 100%) as semi-solid. To a solution of this crude acid (45 g, 167 mmol) and Et₃N (45 g, 440 mmol) in toluene (600 mL) was added DPPA (58 g, 211 mmol) and 2-methylpropan-2-ol (40 g, 352 mmol), and the reaction mixture was heated at reflux overnight. The resulting mixture was diluted with EtOAc (1 L) and water (500 mL). The organic layer was separated and concentrated to give the residue, which was purified by column chromatography (PE:EA=10:1) to give 1-benzyl-3-(tert-butyloxycarbonylamino)-4-methylpyrrolidine as a white solid (12 g, 23.5%): ¹H NMR (400 MHz, CDCl₃) δ ppm 1.1 (d, 3H), 1.4-1.5 (s, 9H), 1.8-2.1 (m, 2H), 2.6-2.7 (d, 2H), 2.9-3.1 (m, 1H), 3.7 (s, 2H), 3.8 (b, 1H), 4.8 (b, 1H), 7.2-7.4 (m, 5H); LCMS m/z 291 (M+H)⁺.

### Intermediate 36: 1-benzyl-3-(N-methyl-tert-butyloxycarbonylamino)-4-methylpyrrolidine

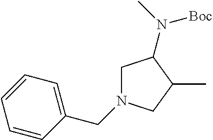

To a solution of 1-benzyl-3-(tert-butyloxycarbonylamino)-4-methylpyrrolidine (2 g, 6.89 mmol) in anhydrous THF (50 mL) was added LAH (0.4 g, 10.33 mmol) at 0° C. under nitrogen atmosphere, and the reaction mixture was heated to reflux for 3 hours. TLC showed the reaction was complete. The reaction mixture was cooled to room temperature, and water (0.4 mL), then aqueous NaOH (15%, 0.6 mL), then water (1.2 mL) were added. The resulting mixture was filtered, and the filtrate was cooled to 0° C. before Boc₂O (1.8 g, 8.26 mmol) was added. After the reaction solution was stirred at temperature for a further 3 h, the solvent was removed in vacuo, and the residue was diluted with aqueous NaOH (2M, 20 mL) and CH₂Cl₂ (50 mL). The organic layer was separated, washed with brine (10 mL), dried over Na₂SO₄, and concentrated. The residue was purified by column chromatography (PE:EA=10:1) to give 1-benzyl-3-(N-methyl-tert-butyloxycarbonylamino)-4-methylpyrrolidine as a colorless oil (1.6 g, 76%): ¹H NMR (400 MHz, CDCl₃) δ ppm 1.0 (d, 3H), 1.43 (s, 9H), 1.8-1.9 (m, 1H), 2.1 (m, 1H), 2.5 (m, 1H), 2.6 (m, 1H), 2.7-2.8 (m, 3H), 2.9-3.0 (m, 1H), 3.4 (d, 1H), 3.6 (d, 1H), 7.2-7.4 (m, 5H); LCMS m/z 304 (M+H)⁺.

### Intermediate 37: 3-(N-methyl-tert-butyloxycarbonylamino)-4-methylpyrrolidine

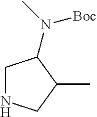

A mixture of 1-benzyl-3-(N-methyl-tert-butyloxycarbonylamino)-4-methylpyrrolidine (0.5 g, 1.64 mmol) and Pd(OH)₂/C (0.1 g) in EtOH (10 mL) was stirred under H₂ (30 psi) for 5 h. After TLC showed that the reaction was completed, the reaction mixture was filtered, and the filtrate was concentrated in vacuo to give 3-(N-methyl-tert-butyloxycarbonylamino)-4-methylpyrrolidine as colorless oil (0.3 g, 80%): ¹H NMR (400 MHz, CDCl₃) δ ppm 1.0 (d, 3H), 1.43 (s, 9H), 2.1 (m, 3H), 2.6 (m, 1H), 2.7-2.8 (m, 4H), 3.1 (m, 1H), 4.2 (b, 1H).

### Intermediate 38: 1-(tert-butyloxycarbonyl)-3-(methoxycarbonylamino)pyrrolidine

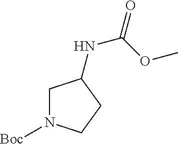

To a mixture of 1-(tert-butyloxycarbonyl)-3-aminopyrrolidine (650 mg, 3.49 mmol) and Et₃N (1.06 g, 10.47 mmol) in dry DCM (10 mL) was added methyl chloroformate (461 mg, 4.89 mmol) dropwise. After being stirred at room temperature for 2 h, the mixture was diluted with 50 mL of DCM. The mixture was washed with H₂O (20 mL) and brine (20 mL), dried over MgSO₄, and concentrated to give 1-(tert-butyloxycarbonyl)-3-(methoxycarbonylamino)pyrrolidine (630 mg, yield 74.0%), which was used for the next step without further purification: ¹H NMR (400 MHz, MeOH-d₄) ppm 4.08 (q, J=5.2 Hz, 1H), 3.61 (s, 3H), 3.50-3.54 (m, 1H), 3.28-3.31 (m, 2H), 3.12-3.17 (m, 1H), 2.03-2.11 (m, 1H), 1.75-1.83 (m, 1H), 1.44 (s, 9H).

### Intermediate 39: 3-(methoxycarbonylamino)pyrrolidine

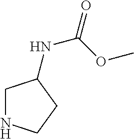

A solution of 1-(tert-butyloxycarbonyl)-3-(methoxycarbonylamino)pyrrolidine (630 mg, 2.57 mmol) in HCl/MeOH (4N, 2 mL) was stirred at room temperature for 1 h. After TLC showed the starting material was completely consumed, the solvent was removed to give 3-(methoxycarbonylamino)pyrrolidine (380 mg, 100%), which was used in the next step without further purification: ¹H NMR (400 MHz, MeOH-d₄) δ ppm 4.19-4.25 (m, 1H), 3.63 (s, 3H), 3.43-3.45 (m, 2H), 3.34-3.38 (m, 1H), 3.14-3.17 (m, 1H), 2.21-2.30 (m, 1H), 1.96-2.03 (m, 1H).

### Intermediate 40:1-(benzyloxycarbonyl)piperidin-4-one

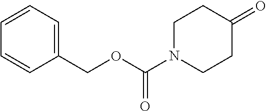

To a 4 N HCl/MeOH solution (100 mL) was added 1-(tert-butyloxycarbonyl)piperidin-4-one (10 g, 50.22 mmol) at 0° C., and the resulting mixture was stirred at room temperature overnight. The mixture was concentrated under reduced pressure to give crude piperidin-4-one hydrochloride (6.78 g, 100%), which was used in the next step without further purification. To a solution of piperidin-4-one hydrochloride (6.78 g, 50.22 mmol) and K₂CO₃ (18.02 g, 130.57 mmol) in 1,4-dioxane (60 mL) and water (60 mL) was added benzyl chloroformate (9.39 g, 55.24 mmol) dropwise at 0°. After addition, the reaction mixture was stirred at room temperature for 18 h, at which time TLC analysis showed the completion of the reaction. The reaction mixture was then concentrated under reduced pressure, and the residue was extracted with EtOAc (3×40 mL). The combined organic layers were dried (Na₂SO₄) and evaporated to give 1-(benzyloxycarbonyl)piperidin-4-one (11.7 g, 99%): ¹H NMR (400 MHz, CDCl₃) ppm 7.30-7.382 (m, 5H), 5.170 (s, 2H), 3.79-3.81 (m, 3H), 2.45 (s, 2H).

### Intermediate 41: 1-(benzyloxycarbonyl)-4-methylaminopiperidine

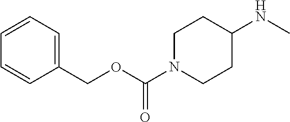

To a suspension of 1-(benzyloxycarbonyl)piperidin-4-one (4 g, 17.16 mmol), methanamine hydrochloride (1.95 g, 18.88 mmol) and triacetoxy sodium borohydride (5.09 g, 24.02 mmol) in DCE (46 mL) was added HOAc (0.8 mL, 12.7 mmol), and the resulting mixture was stirred at room temperature overnight. The reaction mixture was treated with saturated aqueous NaHCO₃ (50 mL), extracted with CH₂Cl₂ (3×40 mL), dried over Na₂SO₄, filtered, and concentrated in vacuo to give crude 1-(benzyloxycarbonyl)-4-methylaminopiperidine (4 g, 100%): ¹H NMR (400 MHz, CDCl₃) δ ppm 7.31-7.35 (m, 5H), 5.12 (s, 2H), 4.13 (s, 2H), 3.72 (s, 3H), 2.86 (t, J=10 Hz, 1H), 2.56-2.63 (m, 1H), 2.48 (s, 3H), 2.12 (s, 3H), 1.91 (s, 1H), 1.22-1.39 (m, 2H).

### Intermediate 42: 1-(benzyloxycarbonyl)-4-(N-methyl-tert-butyloxycarbonylamino)piperidine

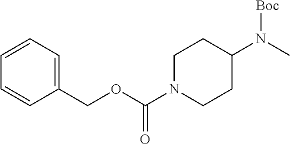

To a solution of 1-(benzyloxycarbonyl)-4-methylaminopiperidine (0.5 g, 2.01 mmol) and di-tert-butyl dicarbonate (483.39 mg, 2.21 mmol) in anhydrous CH₂Cl₂ (10 mL) was added NEt₃ (611.25 mg, 6.04 mmol), and the mixture was stirred at room temperature overnight. The reaction mixture was concentrated in vacuo to give the crude product, which was purified by column chromatography, to give 1-(benzyloxycarbonyl)-4-(N-methyl-tert-butyloxycarbonylamino)piperidine (0.7 g, 51%): ¹H NMR (400 MHz, CDCl₃) δ ppm 7.25-7.30 (m, 5H), 5.05 (s, 2H), 2.63 (s, 3H), 1.56 (s, 9H), 1.48 (s, 9H).

### Intermediate 43: 4-(N-methyl-tert-butyloxycarbonylamino)piperidine

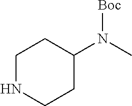

To a solution of 1-(benzyloxycarbonyl)-4-(N-methyl-tert-butyloxycarbonylamino)piperidine (0.35 g, 1 mmol) in anhydrous MeOH (20 mL) was added Pd(OH)₂ (14.11 mg, 0.1 mmol), and the resulting mixture was stirred at room temperature for 2 h under H₂. After TLC showed the stating material was consumed, the solvent was filtered through silica gel, and the filtrate solvent was removed in vacuo to give crude 4-(N-methyl-tert-butyloxycarbonylamino)piperidine (0.2 g, 60%), which was used in the next step without further purification: ¹H NMR (400 MHz, CDCl₃) δ ppm 3.08 (d, J=12.4 Hz, 2H), 2.67 (s, 3H), 2.65 (t, J=14.2 Hz, 2H), 1.82 (s, 2H), 1.50-1.57 (m, 3H), 1.39 (s, 9H).

### Intermediate 44: 1-(tert-butyloxycarbonyl)-3-dimethylaminopiperidine

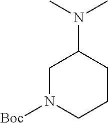

To a suspension of 1-(tert-butyloxycarbonyl)piperidin-3-one (2 g, 10.04 mmol), methanamine hydrochloride (497 mg, 11.04 mmol), and triacetoxy sodium borohydride (2.98 g, 14.06 mmol) in DCE (23 mL) was added HOAc (0.4 mL, 7 mmol), and the resulting mixture was stirred at room temperature overnight. The reaction mixture was treated with aqueous saturated NaHCO₃ (30 mL), extracted with CH₂Cl₂ (3×30 mL), dried over Na₂SO₄, filtered, and concentrated in vacuo to give crude 1-(tert-butyloxycarbonyl)-3-dimethylaminopiperidine (2 g, 88%): ¹H NMR (400 MHz, CDCl₃) δ 4.01-4.23 (m, 2H), 3.81-3.84 (m, 1H), 3.00-3.04 (m, 1H), 3.62-2.74 (m, 31), 2.26 (s, 3H), 1.72-1.83 (m, 3H), 1.47 (s, 9H), 1.34-1.45 (m, 2H).

### Intermediate 45: 3-dimethylaminopiperidine dihydrochloride

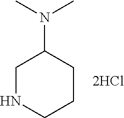

To a HCl/MeOH solution (4N, 50 mL) was added 1-(tert-butyloxycarbonyl)-3-dimethylaminopiperidine (1 g, 4.38 mmol) at 0° C., and the resulting mixture was stirred at room temperature for 0.5 h. The mixture was concentrated under reduced pressure to give crude 3-dimethylaminopiperidine dihydrochloride (1 g, 100%): ¹H NMR (400 MHz, CDCl₃) δ ppm 3.91 (s, 1H), 3.31-3.35 (m, 2H), 3.14-3.23 (m, 6H), 3.00 (m, 2H), 1.98-2.04 (m, 2H), 1.72-1.81 (m, 2H).

### Intermediate 46: 1-(tert-butyloxycarbonyl)-3-methylaminopyrrolidine

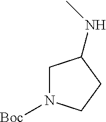

To a solution of 1-(tert-butyloxycarbonyl)pyrrolidin-3-one (74 g, 0.4 mol) in MeOH (450 mL) was added an alcohol solution of MeNH₂ (137.8 g, 1.2 mol) at 0° C., and the resulting mixture was stirred at room temperature for 2 h. Then, NaBH₄ (15.2 g, 0.4 mol) was added, and this mixture was stirred at room temperature for another 1 h, then concentrated under reduced pressure to give the residue. This residue was partitioned between CH₂Cl₂ (3×500 mL) and water (300 mL). The combined organic layers were dried (Na₂SO₄), and evaporated to afforded 1-(tert-butyloxycarbonyl)-3-methylaminopyrrolidine (74 g, 92.5%), which was used in the next step without purification: ¹H NMR (400 MHz, CDCl₃) δ ppm 3.54 (m, 4H), 3.29 (m, 2H), 2.45 (s, 3H), 2.20 (m, 1H), 1.38 (s, 9H).

### Intermediate 47: 1-(tert-butyloxycarbonyl)-3-(N-methylacetamido)pyrrolidine

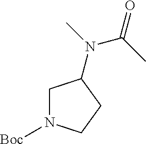

To a cooled (0° C.) solution 1-(tert-butyloxycarbonyl)-3-methylaminopyrrolidine (74 g, 0.37 mol) in CH₂Cl₂ (500 mL) was added Et₃N (74.7 g, 0.74 mol) and acetyl chloride (43.6 g, 0.56 mol). After addition, the reaction mixture was stirred at room temperature for 2 h. The reaction mixture was then diluted with H₂O (200 mL), and extracted with CH₂Cl₂ (3×300 mL). The combined organic layers were dried (Na₂SO₄) and evaporated. The residue was purified by column chromatography, eluting with EtOAc, then EtOAc/CH₃OH (10:1), to give 1-(tert-butyloxycarbonyl)-3-(N-methylacetamido)pyrrolidine (45 g, 51%): ¹H NMR (400 MHz, MeOH-d₄) δ ppm 3.53 (dd, J=10.8 Hz, 2H), 3.33 (m, 2H), 2.87 (d, J=12.4 Hz, 3H), 2.10 (s, 3H), 1.92 (m, 1H), 1.86 (m 1H), 1.46 (s, 9H).

### Intermediate 48: 3-(N-methylacetamido)pyrrolidine

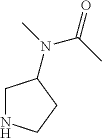

To a solution of 1-(tert-butyloxycarbonyl)-3-(N-methylacetamido)pyrrolidine (6.1 g, 24.8 mmol) in MeOH (20 mL) was added HCl/MeOH (4M, 20 mL) at 0° C. The mixture was stirred at room temperature for 1 h. The solvent was removed in vacuo to give the crude product (4.1 g, 93%), which was used for the next step without further purification: ¹H NMR (400 MHz, MeOH-d₄) δ ppm 4.57 (m, 1H), 3.59 (m, 1H), 3.41 (d, J=3.2 Hz, 2H), 3.23 (m, 1H), 3.07 (s, 3H), 2.33 (m, 1H), 2.16 (m, 1H), 2.10 (s, 3H).

### II. Preparation of Compounds of the Invention

### Example 1

### 4-{[(5-chloro-2-pyridinyl)methyl]oxy}-6′-[3-(dimethylamino)-1-pyrrolidinyl]-2H-1,3′-bipyridin-2-one

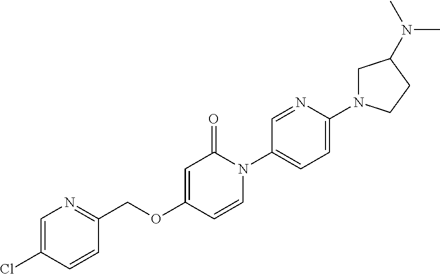

A mixture of 4-{[(5-chloro-2-pyridinyl)methyl]oxy}-2(1H)-pyridinone (approximately 200 mg, 0.8 mmol), 1-(5-bromo-2-pyridinyl)-N,N-dimethyl-3-pyrrolidinamine (approximately 229 mg, 0.8 mmol), trans-cyclohexane-1,2-diamine (96 mg, 0.8 mmol), CuI (161 mg, 0.8 mmol) and K₂CO₃ (350 mg, 2.5 mmol) in 1,4-dioxane (20 mL) was degassed several times and flushed with argon. This mixture was heated at 130° C. for 15 h, at which time TLC analysis showed the completion of the reaction. The solvent was removed under reduced pressure, and the residue was purified by preparative HPLC (eluting with MeCN/water with 0.1% NH₃—H₂O) to afford the title compound (35 mg, 10%): ¹H NMR (400 MHz, CDCl₃) δ ppm 8.53 (d, J=2.00 Hz, 1H), 8.02 (d, J=2.40 Hz, 1H), 7.68 (dd, J=8.40, 2.40 Hz, 1H), 7.45 (dd, J=9.20, 2.40 Hz, 1H), 7.39 (d, J=8.40 Hz, 1H), 7.17 (d, J=7.60 Hz, 1H) 6.36 (d, J=9.20 Hz, 1H), 6.04 (dd, J=7.60, 2.40 Hz, 1H), 5.97 (d, J=2.00 Hz, 1H), 5.11 (s, 2H), 3.76 (t, J=8.80 Hz, 1H), 3.63 (t, J=8.80 Hz, 1H), 3.39 (q, J=7.20 Hz, 1H), 3.26 (t, J=8.80 Hz, 1H), 2.75-2.90 (m, 1H), 2.30 (s, 6H), 2.18-2.28 (m, 1H), 1.88-1.95 (m, 1H); ES-LCMS m/z 426 (M+H)⁺.

### Example 2

### 4-{[(5-chloro-2-pyridinyl)methyl]oxy}-6′-[(3R)-3-(dimethylamino)-1-pyrrolidinyl]-2H-1,3′-bipyridin-2-one

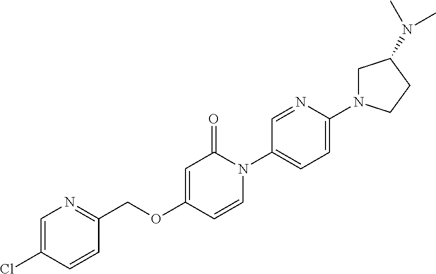

4-{[(5-chloro-2-pyridinyl)methyl]oxy}-6′-(fluoro)-2H-1,3′-bipyridin-2-one (100 mg, 0.3 mmol), (3R)—N,N-dimethyl-3-pyrrolidinamine (40 mg, 0.346 mmol), and K₂CO₃ (120 mg, 0.9 mmol) were dissolved in DMF (2 mL), and the mixture was stirred at 110° C. for 18 h. After LCMS showed that the stating material was consumed, the solvent was removed in vacuo to give the crude product, which was purified by HPLC to afford 4-{[(5-chloro-2-pyridinyl)methyl]oxy}-6′-[(3R)-3-(dimethylamino)-1-pyrrolidinyl]-2H-1,3′-bipyridin-2-one (24.42 mg, 19.1%): ¹H NMR (400 MHz, MeOH-d₄) δ ppm 8.48 (s, 1H), 8.05 (s, 1H), 7.83 (dd, J=8.40 Hz, 2.40 Hz, 1H), 7.58 (d, J=8.40 Hz, 1H), 7.49 (d, J=8.40 Hz, 1H), 7.43 (d, J=7.60 Hz, 1H), 6.67 (d, J=7.60 Hz, 1H), 6.23 (dd, J=7.60 Hz, 2.4 Hz, 1H), 5.99 (s, 1H), 5.15 (s, 2H), 3.94-3.99 (m, 2H), 3.47-3.66 (m, 2H), 3.45-3.50 (m, 1H), 2.90 (s, 6H), 2.45-2.60 (m, 1H), 2.20-2.35 (m, 1H); LCMS m/z 426 (M+H)⁺.

### Example 3

### 4-{[(5-chloro-2-pyridinyl)methyl]oxy}-6′-[(3S)-3-(dimethylamino)-1-pyrrolidinyl]-2H-1,3′-bipyridin-2-one

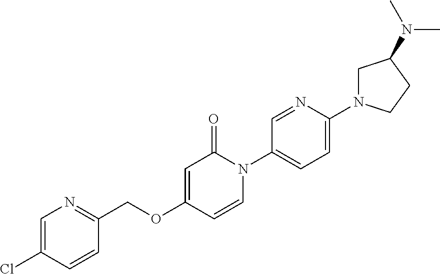

4-{[(5-chloro-2-pyridinyl)methyl]oxy}-6′-(fluoro)-2H-1,3′-bipyridin-2-one (100 mg, 0.3 mmol), (3S)—N,N-dimethyl-3-pyrrolidinamine (40 mg, 0.346 mmol), and K₂CO₃ (120 mg, 0.9 mmol) were dissolved in DMF (2 mL), and the mixture was stirred at 110° C. for 18 h. After LCMS showed that the stating material was comsumed, the solvent was removed in vacuo to give the crude product, which was purified by HPLC to afford 4-{[(5-chloro-2-pyridinyl)methyl]oxy}-6′-[(3S)-3-(dimethylamino)-1-pyrrolidinyl]-2H-1,3′-bipyridin-2-one (4.85 mg, 3.78%): ¹H NMR (400 MHz, MeOH-d₄) δ ppm 8.49 (s, 1H), 8.00 (s, 1H), 7.83 (dd, J=8.40 Hz, 2.40 Hz, 1H), 7.56 (dd, J=8.80 Hz, 2.4 Hz, 1H), 7.49 (d, J=8.40 Hz, 1H), 7.43 (d, J=7.60 Hz, 1H), 6.64 (d, J=9.20 Hz, 1H), 6.23 (dd, J=7.60 Hz, 2.4 Hz, 1H), 5.99 (s, 1H), 5.15 (s, 2H), 3.93-3.96 (m, 2H), 3.21-3.22 (m, 2H), 3.20-3.21 (m, 1H), 2.90 (s, 6H), 2.45-2.60 (m, 1H), 2.20-2.35 (m, 1H); LCMS m/z 426 (M+H)⁺.

### Example 4

### 4-{[(5-chloro-2-pyridinyl)methyl]oxy}-6′-[3-(methylamino)-1-pyrrolidinyl]-2H-1,3′-bipyridin-2-one

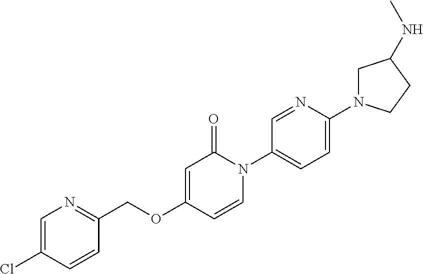

A mixture of 4-{[(5-chloro-2-pyridinyl)methyl]oxy}-2(1H)-pyridinone (approximately 200 mg, 0.8 mmol), 1-(5-bromo-2-pyridinyl)-N-methyl-3-pyrrolidinamine (approximately 217 mg, 0.8 mmol), trans-cyclohexane-1,2-diamine (96 mg, 0.8 mmol), CuI (161 mg, 0.8 mmol) and K₂CO₃ (350 mg, 2.5 mmol) in 1,4-dioxane (20 mL) was degassed several times, and then flushed with argon. This mixture was heated at 130° C. for 15 h, at which time TLC analysis showed the completion of the reaction. The solvent was removed under reduced pressure, and the residue was purified by preparative HPLC (eluting with MeCN/water with 0.1% NH₃—H₂O) to afford the target compound (60 mg, 18%): ¹H NMR (400 MHz, MeOH-d₄) δ ppm 8.59 (s, 1H), 8.14 (d, J=2.40 Hz, 1H), 7.89 (m, 2H), 7.58 (d, J=8.40 Hz, 1H), 7.53 (d, J=8.00 Hz, 1H), 7.01 (d, J=9.60 Hz, 1H), 6.32 (dd, J=7.60, 2.40 Hz, 1H), 6.07 (d, J=2.40 Hz, 1H), 5.23 (s, 2H), 3.68-4.08 (m, 5H), 2.80 (s, 3H), 2.52-2.62 (m, 1H), 2.32-2.41 (m, 1H); ES-LCMS m/z 412 (M+H)⁺.

### Example 5

### 4-{[(5-chloro-2-pyridinyl)methyl]oxy}-6′-[(3R)-3-(methylamino)-1-pyrrolidinyl]-2H-1,3′-bipyridin-2-one

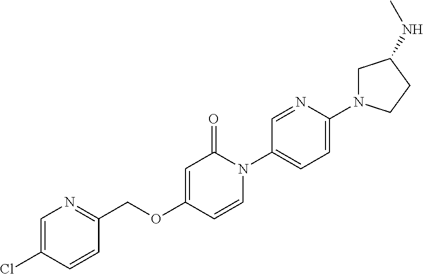

4-{[(5-chloro-2-pyridinyl)methyl]oxy}-6′-(fluoro)-2H-1,3′-bipyridin-2-one (50 mg, 0.15 mmol), (3R)—N-methyl-3-pyrrolidinamine (16.6 mg, 0.16 mmol), and K₂CO₃ (41.6 mg, 0.30 mmol) were dissolved in DMF (2 mL), and the mixture was stirred at 110° C. for 18 h. After LCMS showed that the stating material was consumed, the solvent was removed in vacuo to give the crude product, which was purified by HPLC to afford 4-{[(5-chloro-2-pyridinyl)methyl]oxy}-6′-[(3R)-3-(methylamino)-1-pyrrolidinyl]-2H-1,3′-bipyridin-2-one (12.09 mg, 20%): ¹H NMR (400 MHz, MeOH-d₄) δ ppm 8.56 (s, 1H), 8.09 (s, 1H), 7.90 (dd, J=2.00 Hz, 1H), 7.67 (dd, J=8.00 Hz, 1H), 7.58 (dd, J=7.60 Hz, 1H), 7.51 (dd, J=8.40 Hz, 1H), 6.76 (dd, J=8.80, 1H), 6.32 (dd, J=7.60, 1H), 6.07 (s, 1H), 5.23 (s, 2H), 3.99-4.00 (m, 1H), 3.88-3.93 (m, 1H), 3.73-3.81 (m, 2H), 3.62-3.64 (m, 1H), 2.80 (s, 3H), 2.50-2.58 (m, 1H), 2.22-2.33 (m, 1H); LCMS m/z 411 (M+H)⁺.

### Example 6

### 4-{[(5-chloro-2-pyridinyl)methyl]oxy}-6′-[(3S)-3-(methylamino)-1-pyrrolidinyl]-2H-1,3′-bipyridin-2-one

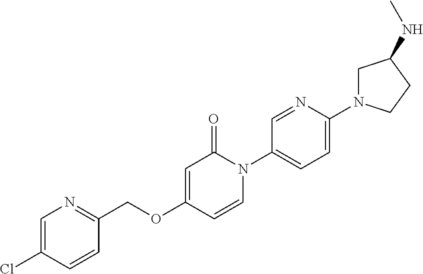

4-{[(5-chloro-2-pyridinyl)methyl]oxy}-6′-(fluoro)-2H-1,3′-bipyridin-2-one (50 mg, 0.15 mmol), (3S)—N-methyl-3-pyrrolidinamine (16.6 mg, 0.16 mmol), and K₂CO₃ (41.6 mg, 0.30 mmol) was dissolved in DMF (2 mL), and the mixture was stirred at 110° C. for 18 h. After LCMS showed that the stating material was consumed, the solvent was removed in vacuo to give the crude product, which was purified by HPLC to afford 4-{[(5-chloro-2-pyridinyl)methyl]oxy}-6′-[(3S)-3-(methylamino)-1-pyrrolidinyl]-2H-1,3′-bipyridin-2-one (17.5 mg, 31%): ¹H NMR (400 MHz, MeOH-d₄) δ ppm 8.56 (s, 1H), 8.09 (s, 1H), 7.90 (dd, J=2.00 Hz, 1H), 7.67 (dd, J=8.00 Hz, 1H), 7.58 (dd, J=7.60 Hz, 1H), 7.51 (dd, J=8.40 Hz, 1H), 6.76 (dd, J=8.80, 1H), 6.32 (dd, J=7.60, 1H), 6.07 (s, 1H), 5.23 (s, 2H), 3.99-4.00 (m, 1H), 3.88-3.93 (m, 1H), 3.73-3.81 (m, 2H), 3.62-3.64 (m, 1H), 2.80 (s, 3H), 2.50-2.58 (m, 1H), 2.22-2.33 (m, 1H); LCMS m/z 411 (M+H)⁺.

### Example 7

### 4-{[(5-chloro-2-pyridinyl)methyl]oxy}-6′-[3-(N-methyl-tert-butyloxycarbonylamino)-4-methyl-1-pyrrolidinyl]-2H-1,3′-bipyridin-2-one

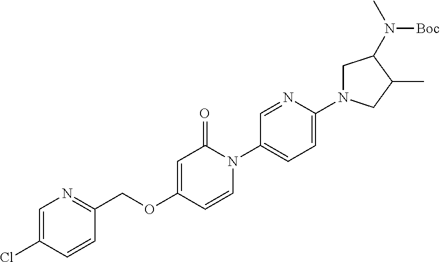

A mixture of 3-(N-methyl-tert-butyloxycarbonylamino)-4-methylpyrrolidine (0.2 g, 0.9 mmol), 4-{[(5-chloro-2-pyridinyl)methyl]oxy}-6′-(fluoro)-2H-1,3′-bipyridin-2-one (0.2 g, 0.6 mmol), and K₂CO₃ (0.17 g, 1.2 mmol) in dry DMF (3 mL) was stirred at 110° C. overnight. After LCMS showed the reaction completed, the reaction mixture was filtered and the filtrate was purified by preparative HPLC to give a sample amount of compound 4-{[(5-chloro-2-pyridinyl)methyl]oxy}-6′-[3-(N-methyl-tert-butyloxycarbonylamino)-4-methyl-1-pyrrolidinyl]-2H-1,3′-bipyridin-2-one as brown oil (40 mg, 12.7%): ¹H NMR (400 MHz, MeOH-d₄) δ ppm 1.1 (b, 1H), 1.4 (s, 9H), 2.8 (s, 3H), 1.98 (m, 5H), 3.4 (m, 1H), 3.5 (m, 1H), 37 (m, 1H), 3.9 (m, 1H), 5.2 (s 2H), 6.1 (s, 1H), 6.4 (m, 1H), 7.1 (m, 1H), 7.5-7.6 (m, 2H), 7.9 (m, 1H), 8.1 (s, 1H), 8.6 (s, 1H); LCMS m/z 526 (M+H)⁺.

### Example 8

### 4-{[(5-chloro-2-pyridinyl)methyl]oxy}-6′-[3-(N-methylamino)-4-methyl-1-pyrrolidinyl]-2H-1,3′-bipyridin-2-one

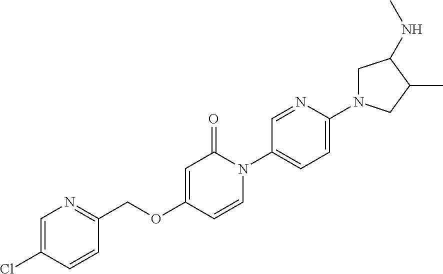

4-{[(5-chloro-2-pyridinyl)methyl]oxy}-6′-[3-(N-methyl-tert-butyloxycarbonylamino)-4-methyl-1-pyrrolidinyl]-2H-1,3′-bipyridin-2-one (40 mg, 0.076 mmol) was dissolved in a solution of TFA in CH₂Cl₂ (20%, 5 mL) at 0° C., and the mixture was stirred at room temperature for 1 h. After LCMS showed that the reaction was complete, the reaction solution was concentrated in vacuo to give 4-{[(5-chloro-2-pyridinyl)methyl]oxy}-6′-[3-(N-methylamino)-4-methyl-1-pyrrolidinyl]-2H-1,3′-bipyridin-2-one (10 mg, 31%) as a brown oil: ¹H-NMR (400 MHz, MeOH-d₄) δ ppm 1.2 (d, 3H), 2.6 (m, 1H), 2.7 (s, 1H), 3.4 (m, 1H), 3.6 (m, 1H), 3.7 (m, 1H), 3.9 (m, 1H), 4.0 (m, 1H), 5.2 (s, 2H), 6.0 (s, 1H), 6.3 (m, 1H), 6.8 (m, 1H), 7.5 (d, 1H), 7.6 (m, 1H), 7.7 (m, 1H), 7.8 (m, 1H), 8.1 (s, 1H), 8.5 (s, 1H); LCMS m/z 426 (M+H)⁺.

### Example 9

### 4-{[(5-chloro-2-pyridinyl)methyl]oxy}-6′-[3-(ethylamino)-1-pyrrolidinyl]-2H-1,3′-bipyridin-2-one

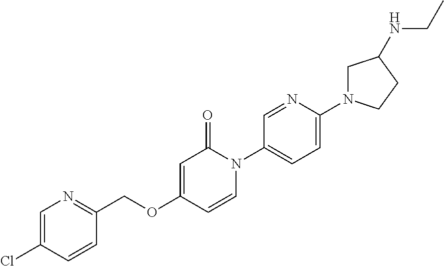

A mixture of 4-{[(5-chloro-2-pyridinyl)methyl]oxy}-2(1H)-pyridinone (approximately 200 mg, 0.8 mmol), 1-(5-bromo-2-pyridinyl)-N-ethyl-3-pyrrolidinamine (approximately 228 mg, 0.8 mmol), trans-cyclohexane-1,2-diamine (96 mg, 0.8 mmol), CuI (161 mg, 0.8 mmol) and K₂CO₃ (350 mg, 2.5 mmol) in 1,4-dioxane (20 mL) was degassed several times and flushed with argon. This mixture was heated at 130° C. for 15 h, at which time TLC analysis showed the completion of the reaction. The solvent was removed under reduced pressure, and the residue was purified by preparative HPLC (eluting with MeCN/water with 0.1% NH₃—H₂O) to afford the title compound (65 mg, 19%): ¹H NMR (400 MHz, MeOH-d₄) δ ppm 8.55 (d, J=2.40 Hz, 1H), 7.96 (d, J=2.80 Hz, 1H), 7.89 (dd, J=8.40, 2.40 Hz, 1H), 7.56 (d, J=8.40 Hz, 1H), 7.49 (dd, J=9.20, 2.40 Hz, 2H), 6.54 (d, J=9.20 Hz, 1H), 6.27 (dd, J=7.60, 2.80 Hz, 1H), 6.04 (d, J=2.40 Hz, 1H), 3.25-3.75 (m, 5H), 2.66-2.72 (m, 2H), 2.20-2.29 (m, 1H), 1.85-1.92 (m, 1H), 1.15 (t, J=7.60 Hz, 3H); ES-LCMS m/z 426 (M+H)⁺.

### Example 10

### 4-{[(5-chloro-2-pyridinyl)methyl]oxy}-6′-[(3R)-3-(ethylamino)-1-pyrrolidinyl]-2H-1,3′-bipyridin-2-one

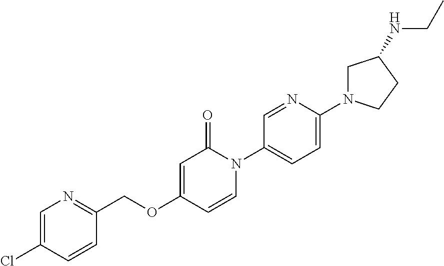

A mixture of 4-{[(5-chloro-2-pyridinyl)methyl]oxy}-2(1H)-pyridinone (88 mg, 0.370 mmol), (3R)-1-(5-bromo-2-pyridinyl)-N-ethyl-3-pyrrolidinamine (100 mg, 0.370 mmol), K₂CO₃ (102 mg, 0.740 mmol), NaI (111 mg, 0.740 mmol), CuI (28.2 mg, 0.148 mmol), and trans-N,N′-dimethylaminocyclohexane (21.1 mg, 0.148 mmol) in 1,4-dioxane (2.5 mL) was degassed under N₂ for 10 minutes. The reaction was sealed then placed in a 155° C. bath and stirred for 15 h. LCMS indicated 80% conversion. The reaction was cooled to 25° C. then poured into EtOAc and 10% aqueous Na₂CO₃. The layers were separated and extracted aqueous with EtOAc. The combined organic layers were washed with brine (1×), dried (Na₂SO₄), filtered, and concentrated in vacuo to a light brown solid. Purified on Agilent reverse phase using CH₃CN/water (w/0.5% TFA) gradient (10:90 to 100:0) over 12 minutes; hv=220 nm) to give product as a TFA salt. Diluted the residue with EtOAc and 1N NaOH and extracted with EtOAc (3×). The combined organics were washed with brine, dried (Na₂SO₄), filtered, and concentrated in vacuo to give the title compound as a white solid (58 mg, 36% yield): ¹H NMR (400 MHz, CDCl₃) δ ppm 8.57 (br s, 1H), 8.04 (br s, 1H), 7.72 (broad dt, J=8.4, 2.4 Hz, 1H), 7.47 (broad dt, J=9.0, 2.7 Hz, 1H), 7.42 (br d, J=8.4 Hz, 1H), 7.21 (br d, J=7.6 Hz, 1H), 6.4 (br d, J=9.0 Hz, 1H), 6.07 (broad dt, J=7.6, 2.7 Hz, 1H), 6.0 (br s, 1H), 5.14 (s, 2H), 3.73 (dd, J=10.2, 6.0 Hz, 1H), 3.68-3.58 (m, 1H), 3.55-3.42 (m, 2H), 3.30 (dd, J=10.0, 5.3 Hz, 1H), 2.73 (q, J=7.1 Hz, 2H), 2.30-2.20 (m, 1H), 1.96-1.85 (m, 1H), 1.15 (t, J=7.1 Hz, 3H); ES-LCMS m/z 426 (M+H)⁺.

An alternative procedure was use for a larger-scale synthesis of the title compound. Thus, a mixture of 4-{[(5-chloro-2-pyridinyl)methyl]oxy}-2(1H)-pyridinone (31.4 g, 133 mmol), (3R)-1-(5-bromo-2-pyridinyl)-N-ethyl-3-pyrrolidinamine (39.4 g, 146 mmol), (1S,2S)—N,N′-dimethyl-1,2-cyclohexanediamine (9.44 g, 66.3 mmol), copper(I) iodide (12.63 g, 66.3 mmol), potassium carbonate (36.7 g, 265 mmol) and sodium iodide (0.994 g, 6.63 mmol) in toluene (700 ml) was purged with nitrogen for 15 min and then heated at reflux for 3 h. HPLC showed -95% completion with ˜1% of 4-iodo analog. To this was added 1 eq (13 g) of CuCl and 3 eq (29 g) of KCl, and the mixture was heated at reflux for 1 hr. The reaction was allowed to stir at RT overnight. The reaction was diluted with DCM (700 mL), 2% aqueous ammonium hydroxide (500 mL) and stirred for 1 hr. The mixture was filtered thru Celite, rinsed with DCM and the layers separated. The organics were washed with 2% aqueous ammonium hydroxide (4×500 mL), treated with magnesium sulfate and darco, filtered thru Celite and concentrated down at 40° C. to −600 mL. The mixture was stirred until the slurry cooled to ambient temperature. The resulting solid was filtered, rinsed with toluene, and dried to afford the desired product as a light beige solid (39.2 g, 69%): ¹H NMR (400 MHz, CDCl₃) δ ppm 8.54 (d, J=2.36 Hz, 1H), 8.02 (d, J=2.57 Hz, 1H), 7.69 (dd, J=8.32, 2.47 Hz, 1H), 7.45 (dd, J=8.94, 2.67 Hz, 1H), 7.40 (d, J=8.32 Hz, 1H), 7.18 (d, J=7.61 Hz, 1H), 6.37 (d, J=8.94 Hz, 1H), 6.04 (dd, J=7.61, 2.67 Hz, 1H), 5.98 (d, J=2.67 Hz, 1H), 5.11 (s, 2H), 3.69 (dd, J=10.28, 6.27 Hz, 1H), 3.59 (ddd, J=9.87, 7.96, 5.70 Hz, 1H), 3.39-3.51 (m, 2H), 3.25 (dd, J=10.28, 5.45 Hz, 1H), 2.69 (q, J=7.13 Hz, 2H), 2.21 (dd, J=12.74, 7.19 Hz, 1H), 1.80-1.91 (m, 1H), 1.11 (t, J=7.09 Hz, 3H); MS m/z 426 (M+H)⁺; HPLC peak RT=1.74 min. The filtrate was concentrated, ethyl acetate (200 mL) was added, and the mixture was stirred for 1 hr. The resulting solid was filtered to afford additional product (6.9 g, 12%, purity ˜94%.).

### Example 11

### 4-{[(5-chloro-2-pyridinyl)methyl]oxy}-6′-[(3S)-3-(ethylamino)-1-pyrrolidinyl]-2H-1,3′-bipyridin-2-one

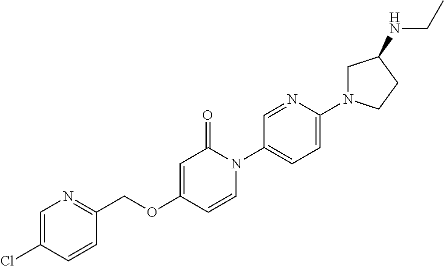

A mixture of 4-{[(5-chloro-2-pyridinyl)methyl]oxy}-2(1H)-pyridinone (88 mg, 0.370 mmol), (3S)-1-(5-bromo-2-pyridinyl)-N-ethyl-3-pyrrolidinamine (100 mg, 0.370 mmol), K₂CO₃ (102 mg, 0.740 mmol), NaI (111 mg, 0.740 mmol), CuI (28.2 mg, 0.148 mmol), and trans-N,N′-dimethylaminocyclohexane (21.06 mg, 0.148 mmol) in 1,4-dioxane (2.5 mL) was degassed under N₂ for 10 min. The reaction was sealed then placed in a 155° C. bath and stirred for 15 h. LCMS indicated 50-60% conversion with significant (3S)—N-ethyl-1-(5-iodo-2-pyridinyl)-3-pyrrolidinamine present. Added additional CuI (28.2 mg, 0.148 mmol) and trans-N,N′-dimethylaminocyclohexane (21.1 mg, 0.148 mmol), then degassed and heated at 155° C. for δ h. LCMS indicated 70% conversion. The reaction was cooled to 25° C. then poured into EtOAc and 10% aqueous Na₂CO₃. The layers were separated layers and extracted aqueous with EtOAc. Combined organic layers were washed with brine (1×), dried (Na₂SO₄), filtered, and concentrated in vacuo to a light brown solid. Purified on Agilent reverse phase using CH₃CN/water (w/0.5% TFA) gradient (10:90 to 100:0) over 12 minutes; hv=220 nm) to give product as a TFA salt. Diluted the residue with EtOAc and 1N NaOH and extracted with EtOAc (3×). The combined organics were washed with brine, dried (Na₂SO₄), filtered, and concentrated in vacuo to give 36 mg (22% yield) of the title compound as a white solid: ¹H NMR (400 MHz, CDCl₃) δ ppm 8.57 (br s, 1H), 8.04 (br s, 1H), 7.72 (broad dt, J=8.4, 2.4 Hz, 1H), 7.47 (broad dt, J=9.0, 2.7 Hz, 1H), 7.42 (br d, J=8.4 Hz, 1H), 7.21 (br d, J=7.6 Hz, 1H), 6.4 (br d, J=9.0 Hz, 1H), 6.07 (broad dt, J=7.6, 2.7 Hz, 1H), 6.0 (br s, 1H), 5.14 (s, 2H), 3.73 (dd, J=10.2, 6.0 Hz, 1H), 3.68-3.58 (m, 1H), 3.55-3.42 (m, 2H), 3.30 (dd, J=10.0, 5.3 Hz, 1H), 2.73 (q, J=7.1 Hz, 2H), 2.30-2.20 (m, 1H), 1.96-1.85 (m, 1H), 1.15 (t, J=7.1 Hz, 3H); ES-LCMS m/z 426 (M+H)⁺.

### Example 12

### 4-{[(5-chloro-2-pyridinyl)methyl]oxy}-6′-[3-(amino)-1-pyrrolidinyl]-2H-1,3′-bipyridin-2-one

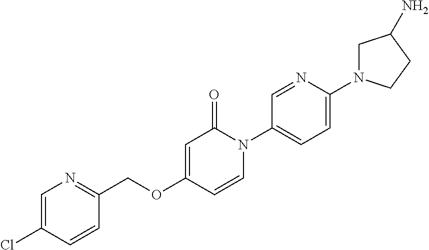

A mixture of 4-{[(5-chloro-2-pyridinyl)methyl]oxy}-2(1H)-pyridinone (approximately 200 mg, 0.8 mmol), 1-(5-bromo-2-pyridinyl)-3-pyrrolidinamine (approximately 205 mg, 0.8 mmol), trans-cyclohexane-1,2-diamine (96 mg, 0.8 mmol), CuI (161 mg, 0.8 mmol) and K₂CO₃ (350 mg, 2.5 mmol) in 1,4-dioxane (20 mL) was degassed several times and flushed with argon. This mixture was heated at 130° C. for 15 h, at which time TLC analysis showed the completion of the reaction. The solvent was removed under reduced pressure, and the residue was purified by preparative HPLC (eluting with MeCN/water with 0.1% NH₃—H₂O) to afford the title compound (30 mg, 10%): ¹H NMR (400 MHz, MeOH-d₄) δ ppm 8.59 (s, 1H), 8.10 (d, J=2.40 Hz, 1H), 7.90 (dd, J=8.40, 1.60 Hz, 1H), 7.80 (dd, J=10.80, 2.40 Hz, 1H), 7.58 (d, J=8.00 Hz, 1H), 7.52 (d, J=7.60 Hz, 1H), 6.90 (d, J=9.20 Hz, 1H), 6.31 (dd, J=12.40, 1.60 Hz, 1H), 6.06 (d, J=2.00 Hz, 1H), 5.22 (s, 2H), 4.09-4.13 (m, 1H), 3.89-3.95 (m, 1H), 3.67-3.79 (m, 3H), 2.49-2.58 (m, 1H), 2.23-2.32 (m, 1H); ES-LCMS m/z 398 (M+H)⁺.

### Example 13

### 4-{[(5-chloro-2-pyridinyl)methyl]oxy}-6′-(1,3′-bipyrrolidin-1′-yl)-2H-1,3′-bipyridin-2-one

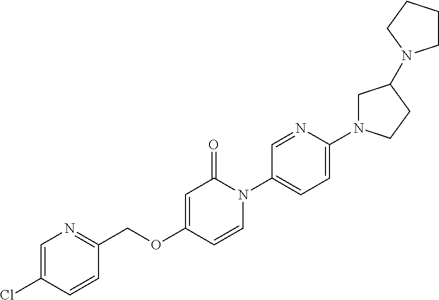

A mixture of 4-{[(5-chloro-2-pyridinyl)methyl]oxy}-2(1H)-pyridinone (approximately 200 mg, 0.8 mmol), 1′-(5-bromo-2-pyridinyl)-1,3′-bipyrrolidine (approximately 251 mg, 0.8 mmol), trans-cyclohexane-1,2-diamine (96 mg, 0.8 mmol), CuI (161 mg, 0.8 mmol) and K₂CO₃ (350 mg, 2.5 mmol) in 1,4-dioxane (20 mL) was degassed several times and flushed with argon. This mixture was heated at 130° C. for 15 h, at which time TLC analysis showed the completion of the reaction. The solvent was removed under reduced pressure, and the residue was purified by preparative HPLC (eluting with MeCN/water with 0.1% NH₃—H₂O) to afford the title compound (20 mg, 6%): ¹H NMR (400 MHz, CDCl₃) δ ppm 8.54 (d, J=2.00 Hz, 1H), 8.02 (d, J=2.80 Hz, 1H), 7.69 (dd, J=8.40, 2.40 Hz, 1H), 7.46 (dd, J=9.20, 2.40 Hz, 1H), 7.40 (d, J=8.40 Hz, 1H), 6.38 (d, J=9.20 Hz, 1H), 6.05 (dd, J=7.20, 2.80 Hz, 1H), 6.98 (d, J=2.80 Hz, 1H), 5.11 (s, 1H), 3.35-3.82 (m, 4H), 2.90-2.96 (m, 1H), 2.60-2.70 (m, 3H), 1.85-2.30 (m, 7H); ES-LCMS m/z 452.4 (M+H)⁺.

### Example 14

### 4-{[(5-chloro-2-pyridinyl)methyl]oxy}-6′-[3-(4-morpholinyl)-1-pyrrolidinyl]-2H-1,3′-bipyridin-2-one

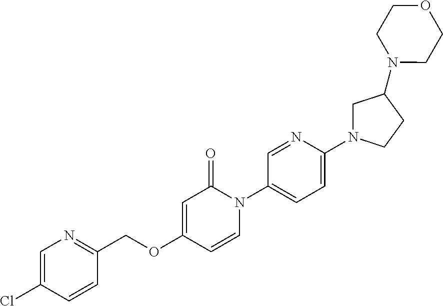

A mixture of 4-{[(5-chloro-2-pyridinyl)methyl]oxy}-2(1H)-pyridinone (approximately 200 mg, 0.8 mmol), 4-[1-(5-bromo-2-pyridinyl)-3-pyrrolidinyl]morpholine (approximately 264 mg, 0.8 mmol), trans-cyclohexane-1,2-diamine (96 mg, 0.8 mmol), CuI (161 mg, 0.8 mmol) and K₂CO₃ (350 mg, 2.5 mmol) in 1,4-dioxane (20 mL) was degassed several times and flushed with argon. This mixture was heated at 130° C. for 15 h at which time TLC analysis showed the completion of the reaction. The solvent was removed under reduced pressure, and the residue was purified by preparative HPLC (eluting with MeCN/water with 0.1% NH₃—H₂O) to afford the title compound (55 mg, 15%): ¹H NMR (400 MHz, CDCl₃) δ ppm 8.53 (d, J=2.00 Hz, 1H), 8.02 (d, J=2.40 Hz, 1H), 7.69 (dd, J=8.40, 2.40 Hz, 1H), 7.45 (dd, J=8.80, 2.40 Hz, 1H), 7.39 (d, J=8.40 Hz, 1H), 7.23 (s, 1H), 7.18 (d, J=7.60 Hz, 1H), 6.37 (d, J=8.80 Hz, 1H), 6.05 (dd, J=7.60, 2.40 Hz, 1H), 5.98 (d, J=2.80 Hz, 1H), 5.11 (s, 2H), 3.79 (t, J=7.20 Hz, 1H), 3.73 (t, J=4.40 Hz, 3H), 3.64 (t, J=9.60 Hz, 1H), 3.36-3.43 (m, 1H), 3.28 (t, J=8.80 Hz, 1H), 2.90-2.99 (m, 1H), 2.49-2.59 (m, 3H), 2.20-2.28 (m, 1H), 1.88-1.96 (m, 3H); ES-LCMS m/z 468 (M+H)⁺.

### Example 15

### 4-{[(5-chloro-2-pyridinyl)oxy]methyl}-6′-[3-(dimethylamino)-1-pyrrolidinyl]2H-1,3′-bipyridin-2-one

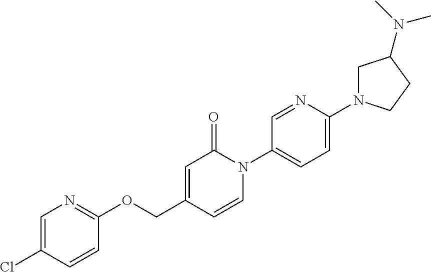

To a 40 mL vial charged with a solution of 6′-[3-(dimethylamino)-1-pyrrolidinyl]4-(hydroxymethyl)-2H-1,3′-bipyridin-2-one (100 mg, 0.318 mmol), PPh₃ (104 mg, 0.398 mmol) and 5-chloro-2(1H)-pyridinone (51.5 mg, 0.398 mmol) in THF (2 mL) at 25° C. was added DIAD (0.077 mL, 0.398 mmol) in THF (2 mL) in a drop wise fashion via syringe over 2 minutes. The reaction was stirred at 25° C. overnight whereupon LCMS analysis confirmed product formation. The reaction was concentrated in vacuo and purified via reverse phase chromatography eluting with a linear 0.5%-50% CH₃CN—H₂O gradient containing 0.1% TFA to provide the title compound as a dark oil (9.4 mg, 5%) of: ¹H NMR (400 MHz, MeOH-d₄) δ ppm 2.26-2.42 (m, 1H), 2.56-2.71 (m, 1H), 2.99 (s, 6H), 3.59 (dt, J=10.5, 8.1 Hz, 1H), 3.71-3.79 (m, 1H), 3.83 (ddd, J=10.4, 8.8, 3.7 Hz, 1H), 4.00-4.14 (m, 2H), 5.33 (d, J=0.9 Hz, 2H), 6.51 (dd, J=7.1, 1.7 Hz, 1H), 6.62 (d, J=1.1 Hz, 1H), 6.82 (d, J=9.2 Hz, 1H), 6.94 (d, J=8.8 Hz, 1H), 7.57 (d, J=7.1 Hz, 1H), 7.74 (ddd, J=8.9, 7.6, 2.6 Hz, 2H), 8.10 (d, J=2.1 Hz, 1H), 8.15 (d, J=2.4 Hz, 1H); EI-LCMS m/z 427 (M+H)⁺.

### Example 16

### 4-{[(6-chloro-3-pyridinyl)oxy]methyl}-6′-[3-(dimethylamino)-1-pyrrolidinyl]-2H-1,3′-bipyridin-2-one

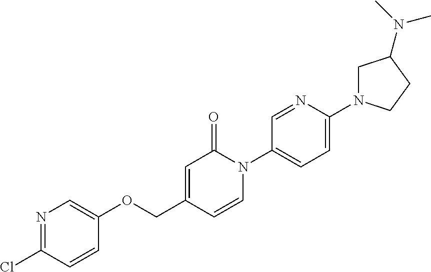

To a 40 mL vial charged with a solution of 6′-[3-(dimethylamino)-1-pyrrolidinyl]-4-(hydroxymethyl)-2H-1,3′-bipyridin-2-one (100 mg, 0.318 mmol), PPh₃ (104 mg, 0.398 mmol) and 6-chloro-3-pyridinol (51.5 mg, 0.398 mmol) in THF (2 mL) at 25° C. was added DIAD (0.077 mL, 0.398 mmol) in THF (2 mL) a drop wise fashion via syringe over 2 minutes. The reaction was stirred at 25° C. overnight whereupon LCMS analysis indicated product formation. The reaction was concentrated in vacuo and purified by medium pressure silica gel chromatography (biotage with 2-30% methanol/DCM as eluent) to provide the title compound as a dark solid (22 mg, 16%): ¹H NMR (400 MHz, MeOH-d₄) δ ppm 1.89-2.03 (m, 1H), 2.28-2.45 (m, 7H), 2.98-3.10 (m, 1H), 3.31-3.36 (m, 1H), 3.45 (td, J=10.1, 7.1 Hz, 1H), 3.66-3.75 (m, 1H), 3.82 (dd, J=10.1, 7.3 Hz, 1H), 5.15 (d, J=0.9 Hz, 2H), 6.52 (dd, J=7.0, 1.8 Hz, 1H), 6.61 (d, J=9.0 Hz, 1H), 6.69 (d, J=1.3 Hz, 1H), 7.39 (d, J=8.8 Hz, 1H), 7.49-7.58 (m, 2H), 7.61 (d, J=7.1 Hz, 1H), 8.04 (d, J=2.1 Hz, 1H), 8.15 (d, J=2.8 Hz, 1H); EI-LCMS m/z 427 (M+H)⁺.

### Example 17

### 4-{[(2-pyridinyl)methyl]oxy}-6′-[3-(dimethylamino)-1-pyrrolidinyl]-2H-1,3-bipyridin-2-one

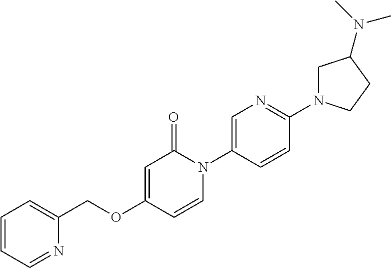

A mixture of 6′-[3-(dimethylamino)-1-pyrrolidinyl]-4-hydroxy-2H-1,3′-bipyridin-2-one (150 mg, 0.499 mmol) and triphenylphosphine (210 mg, 0.799 mmol) in a flask was treated with 2-pyridinylmethanol (65.4 mg, 0.599 mmol) dissolved in dichloromethane (3 ml). Bis(1,1-dimethylethyl) (E)-1,2-diazenedicarboxylate (184 mg, 0.799 mmol) was added in two portions, and the reaction mixture was stirred at 25° C. for 2 h then concentrated under a stream of nitrogen gas. Purification by reverse phase HPLC (1 to 50% gradient), treatment of fractions containing product with MP-carbonate resin, filtration, and concentration provided crude product. The residue was purified by normal phase chromatography using an ISCO amine column (0 to 7% methanol in dichloromethane) to provide the title compound as a white solid (77 mg, 39%): ¹H NMR (400 MHz, MeOH-d₄) δ ppm 8.54 (d, J=4.9 Hz, 1H), 7.97 (d, J=2.4 Hz, 1H), 7.87 (dt, J=7.7, 1.7 Hz, 1H), 7.56 (d, J=7.7 Hz, 1H), 7.52-7.46 (m, 1H), 7.38 (br dd, J=7.5, 5.8 Hz, 1H), 6.56 (d, J=8.8 Hz, 1H), 6.27 (dd, J=7.5, 2.7 Hz, 1H), 6.05 (d, J=2.7 Hz, 1H), 5.21 (s, 2H), 3.85-3.73 (m, 1H), 3.66 (t, J=8.7 Hz, 1H), 3.41 (dd, J=10.3, 6.9 Hz, 1H), 3.33-3.20 (m, 1H), 2.97-2.86 (m, 1H), 2.32 (s, 6H), 2.39-2.23 (m, 1H), 1.98-1.83 (m, 1H); ES-LCMS m/z 392 (M+H)⁺.

### Example 18

### 4-{[(3,5-difluoro-2-pyridinyl)methyl]oxy}-6′-[3-(dimethylamino)-1-pyrrolidinyl]-2H-1,3′-bipyridin-2-one

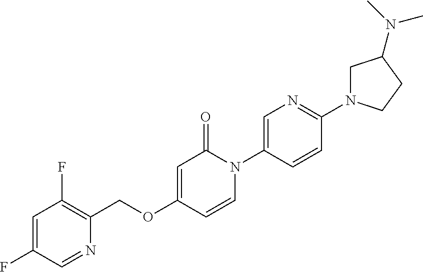

A mixture of 6′-[3-(dimethylamino)-1-pyrrolidinyl]-4-hydroxy-2H-1,3′-bipyridin-2-one (102 mg, 0.340 mmol) and triphenylphosphine (223 mg, 0.849 mmol) was treated with (3,5-difluoro-2-pyridinyl)methanol (59.1 mg, 0.408 mmol) dissolved in dichloromethane (3 ml). Bis(1,1-dimethylethyl) (E)-1,2-diazenedicarboxylate (195 mg, 0.849 mmol) was added in two portions, and the reaction mixture was stirred at 25° C. for 2 h then concentrated under a stream of nitrogen. Purification by reverse phase HPLC (1 to 50% gradient) and concentration of the fractions containing product provided a residue. The residue was treated with aqueous NaHCO₃ solution and extracted with ethyl acetate. The organic layer was washed with brine, dried (Na₂SO₄), filtered, and concentrated in vacuo to give a white solid (65 mg, 45% yield): ¹H NMR (400 MHz, CDCl₃) δ ppm 8.36 (d, J=2.4 Hz, 1H), 8.02 (d, J=2.4 Hz, 1H), 7.46 (dd, J=9.0, 2.6 Hz, 1H), 7.28-7.21 (m, 1H), 7.15 (d, J=7.6 Hz, 1H), 6.37 (d, J=9.0 Hz, 1H), 6.07 (d, J=2.7 Hz, 1H), 5.99 (dd, J=7.6 2.7 Hz, 1H), 5.15 (s, 2H), 3.83-3.73 (m, 1H), 3.64 (t, J=8.9 Hz, 1H), 3.40 (dt, J=10.1, 6.9 Hz, 1H), 3.28 (t, J=9.0 Hz, 1H), 2.91-2.78 (m, 1H), 2.32 (s, 6H), 2.28-2.18 (m, 1H), 2.02-1.88 (m, 1H); ES-LCMS m/z 428 (M+H)⁺.

### Example 19

### 4-{[(4-chloro-5-fluoro-2-pyridinyl)methyl]oxy}-6′-[3-(dimethylamino)-1-pyrrolidinyl]-2H-1,3′-bipyridin-2-one

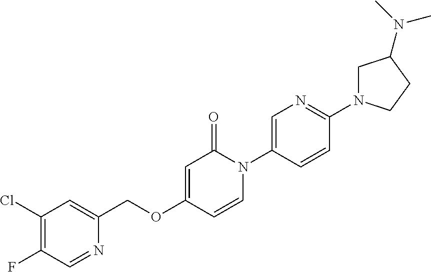

A mixture of 6′-[3-(dimethylamino)-1-pyrrolidinyl]-4-hydroxy-2H-1,3′-bipyridin-2-one (150 mg, 0.499 mmol) and triphenylphosphine (327 mg, 1.249 mmol) was treated with (4-chloro-5-fluoro-2-pyridinyl)methanol (81 mg, 0.499 mmol) dissolved in dichloromethane (5 ml). Bis(1,1-dimethylethyl) (E)-1,2-diazenedicarboxylate (287 mg, 1.249 mmol) was added in two portions, and the reaction mixture was stirred at 25° C. for 2 h then concentrated under a stream of nitrogen gas. Purification by reverse phase HPLC (1 to 50% gradient) and concentration of the fractions containing product provided a residue. The residue was treated with aqueous NaHCO₃ solution and extracted with ethyl acetate. The organic layer was washed with brine, dried (Na₂SO₄), filtered and concentrated in vacuo to provide the title compound as a white solid (114 mg, 51%): ¹H NMR (400 MHz, CDCl₃) δ ppm 8.46 (s, 1H), 8.06 (d, J=2.7 Hz, 1H), 7.56-7.48 (m, 2H), 7.21 (d, J=7.6 Hz, 1H), 6.43 (d, J=9.0 Hz, 1H), 6.09 (dd, J=7.4, 2.5 Hz, 1H), 5.98 (d, J=2.7 Hz, 1H), 5.10 (s, 2H), 3.95-3.84 (m, 1H), 3.77-3.65 (br m, 2H), 3.55-3.22 (br m, 2H), 2.60 (br s, 6H), 2.47-2.29 (br m, 2H); ES-LCMS m/z 444 (M+H)⁺.

### Example 20

### 4-{[(5-fluoro-2-pyridinyl)methyl]oxy}-6′-[3-(dimethylamino)-1-pyrrolidinyl]-2H-1,3′-bipyridin-2-one

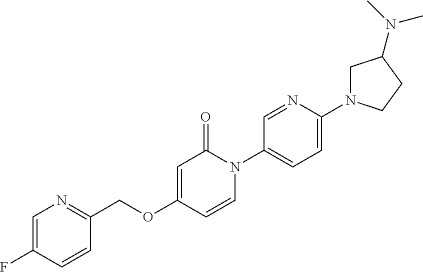

A solution of 6′-[3-(dimethylamino)-1-pyrrolidinyl]-4-hydroxy-2H-1,3′-bipyridin-2-one (100 mg, 0.333 mmol) and triphenylphosphine (140 mg, 0.533 mmol) in dichloromethane (5 ml) was treated with (5-fluoro-2-pyridinyl)methanol (50.8 mg, 0.400 mmol) followed by bis(1,1-dimethylethyl) (E)-1,2-diazenedicarboxylate (123 mg, 0.533 mmol). After stirring overnight, the reaction was treated with additional (5-fluoro-2-pyridinyl)methanol (50.8 mg, 0.400 mmol), triphenylphosphine (140 mg, 0.533 mmol), and bis(1,1-dimethylethyl) (E)-1,2-diazenedicarboxylate (123 mg, 0.533 mmol). After stirring for 2 h, the reaction was concentrated in vacuo and residue purified via Agilent semi-prep HPLC using 5-50% CH₃CN/H₂O. The resulting residue was treated with saturated aqueous NaHCO₃ solution and extracted with EtOAc. The combined extracts were dried (Na₂SO₄), filtered, and concentrated in vacuo to give the title compound (61 mg, 44%): ¹H NMR (400 MHz, CDCl₃) δ ppm 8.46 (s, 1H), 8.05 (d, J=2.7 Hz, 1H), 7.51-7.41 (m, 3H), 7.19 (d, J=7.6 Hz, 1H), 6.39 (d, J=9.0 Hz, 1H), 6.06 (dd, J=7.6, 2.7 Hz, 1H), 6.01 (d, J=2.4 Hz, 1H), 5.13 (s, 2H), 3.79 (t, J=8.3 Hz, 1H), 3.65 (t, J=9.4 Hz, 1H), 3.47-3.36 (m, 1H), 3.33-3.20 (m, 1H), 2.81 (br s, 1H), 2.32 (s, 6H), 2.29-2.18 (m, 1H), 2.01-1.86 (m, 1H); ES-LCMS m/z 410 (M+H)⁺.

### Example 21

### 4-{[(5-chloro-2-pyridinyl)methyl]oxy}-6′-[3-(dimethylamino)-1-pyrrolidinyl]-5′-methyl-2H-1,3′-bipyridin-2-one

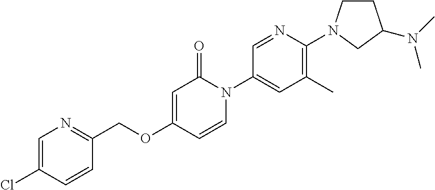

A mixture of 4-{[(5-chloro-2-pyridinyl)methyl]oxy}-2(1H)-pyridinone (83 mg, 0.35 mmol), 1-(5-bromo-3-methyl-2-pyridinyl)-N,N-dimethyl-3-pyrrolidinamine (100 mg, 0.35 mmol), trans-cyclohexane-1,2-diamine (14 mg, 0.1 mmol), CuI (27 mg, 0.14 mmol) and K₂CO₃ (97 mg, 0.70 mmol) in 1,4-dioxane (20 mL) was degassed with Argon. This mixture was sealed and heated at 150° C. for 15 h at which time TLC analysis showed the completion of the reaction. The solvent was removed under reduced pressure, and the residue was purified by preparative HPLC (eluting with MeCN/water with 0.1% NH₃—H₂O) to afford the title compound (25 mg, 15%): ¹H NMR (400 MHz, CDCl₃) δ ppm 2.29-2.38 (m, 1H), 2.48 (s, 3H), 2.55 (dd, J=6.7, 3.8 Hz, 1H), 2.97 (d, J J=2.5 Hz, 1H), 3.00 (s, 6H), 3.76 (d, J=8.4 Hz, 1H), 3.88 (dd, J=10.4, 8.2 Hz, 1H), 3.96-4.08 (m, 2H), 5.23 (d, J=2.2 Hz, 2H), 6.08 (s, J=2.3 Hz, 1H), 6.33 (dd, J=7.4, 2.7 Hz, 1H), 7.52 (dd, J=7.7, 2.8 Hz, 1H), 7.59 (d, J=6.2 HZ, 1H) 7.66 (s, 1H), 7.93 (dd, J=8.4, 2.6 Hz, 1H), 8.04 (s, 1H), 8.58 (s, 1H); ES-LCMS m/z 440 (M+H)⁺.

### Example 22

### 4-{[(5-chloro-2-pyridinyl)methyl]oxy}-6′-{3-[methyl(1-methylethyl)amino]-1-pyrrolidinyl}-2H-1,3′-bipyridin-2-one

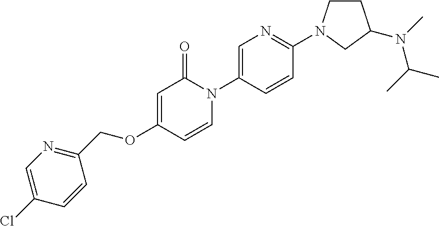

A mixture of 4-{[(5-chloro-2-pyridinyl)methyl]oxy}-2(1H)-pyridinone (71.4 mg, 0.302 mmol), 1-(5-bromo-2-pyridinyl)-N-methyl-N-(1-methylethyl)-3-pyrrolidinamine (90 mg, 0.302 mmol), trans-cyclohexane-1,2-diamine (17.7 mg, 0.121 mmol), CuI (22.9 mg, 0.121 mmol), NaI (90 mg, 0.604 mmol), and K₂CO₃ (83 mg, 0.604 mmol) in 1,4-dioxane (10 mL) was degassed several times and flushed with dry nitrogen. This mixture was heated at 130° C. in a sealed reaction vessel for 15 h at which time LC/MS analysis showed the completion of the reaction. The solvent was removed under reduced pressure, and the residue was purified via flash chromatography (ISCO with 0-15% methanol/EtOAC as eluent) to afford the title compound (34 mg, 25%): ¹H NMR (400 MHz, MeOH-d₄) δ ppm 8.58 (d, J=1.76 Hz, 1H), 7.99 (dd, J=2.30 Hz, 0.29 Hz 1H), 7.91 (s, 1H), 7.60 (d, J=0.73, 1H), 7.50-7.55 (m, 2H), 6.59 (d, J=9.35 Hz, 1H), 6.29 (d, J=7.63, 1H), 6.06-6.08 (m, 1H), 5.24 (s, 2H), 3.79-3.86 (m, 2H), 3.65-3.73 (m, 2H), 3.03-3.17 (m, 3H), 2.18-2.41 (m, 3H), 1.81-1.93 (m, 1H), 1.09 (d, J=0.24 HZ, 6H); ES-LCMS m/z 454 (M+H)⁺.

### Example 23

### 4-{[(5-chloro-2-pyridinyl)methyl]oxy}-6′-{3-[ethyl(methyl)amino]-1-pyrrolidinyl}-2H-1,3′-bipyridin-2-one

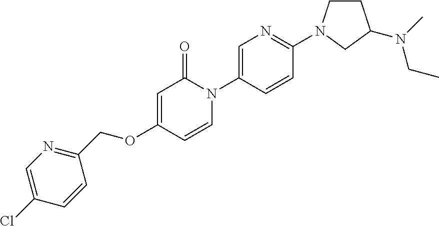

A mixture of 4-{[(5-chloro-2-pyridinyl)methyl]oxy}-2(1H)-pyridinone (71.4 mg, 0.302 mmol), 1-(5-bromo-2-pyridinyl)-N-methyl-N-(1-methylethyl)-3-pyrrolidinamine (90 mg, 0.302 mmol), trans-cyclohexane-1,2-diamine (17.7 mg, 0.121 mmol), CuI (22.9 mg, 0.121 mmol), NaI (90 mg, 0.604 mmol), and K₂CO₃ (83 mg, 0.604 mmol) in 1,4-dioxane (10 mL) was degassed several times and flushed with dry nitrogen. This mixture was heated at 130° C. for 15 h at which time LC/MS analysis showed the completion of the reaction. The solvent was removed under reduced pressure, and the residue purified via flash chromatography (ISCO with 0-15% methanol/EtOAC as eluent) to afford the title compound (34 mg, 25%): ¹H NMR (400 MHz, MeOH-d₄) δ ppm 8.55 (dd, J=2.44, 0.62 Hz, 1H), 8.12 (dd, J=2.55 Hz, 0.51 Hz 1H), 7.89 (dd, J=8.43, 2.47 Hz, 1H), 7.77 (dd, J=9.24, 2.58 Hz 1H), 7.51-7.57 (m, 2H), 6.88 (d, J=0.38 Hz, 1H), 6.30 (dd, J=7.66, 2.71 Hz, 1H), 6.05 (d, J=2.69 Hz, 1H), 5.21 (s, 2H), 4.16 (m, 1H), 4.07 (m, 1H), 3.78 (m, 2H), 3.60 (m, 1H), 2.92 (s, 3H), 3.27 (m, 2H), 2.61 (m, 1H) 2.36 (m, 1H) 1.37 (t, J=7.28 Hz, 3H); ES-LCMS m/z 440 (M+H)⁺.

### Example 24

### 4-{[(5-chloro-2-pyridinyl)methyl]oxy}-6′-[3-(cyclohexylamino)-1-pyrrolidinyl]-2H-1,3′-bipyridin-2-one

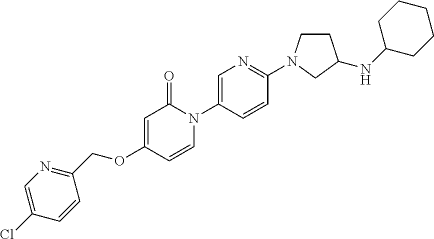

1-(5-bromo-2-pyridinyl)-N-cyclohexyl-3-pyrrolidinamine (137 mg, 0.423 mmol) and 4-{[(5-chloro-2-pyridinyl)methyl]oxy}-2(1H)-pyridinone (100 mg, 0.423 mmol) was allowed to stir and dissolve in 1,4-dioxane (10 mL). Next, NaI (127 mg, 0.845 mmol), K₂CO₃ (117 mg, 0.845 mmol), and CuI (32.2 mg, 0.169 mmol) were added and the reaction mixture flushed with dry nitrogen for 15 min. Trans-cyclohexane-1,2-diamine (24.5 mg, 0.169 mmol) was then added and the reaction vessel sealed. The reaction was allowed to stir and heat at 130° C. for 15 h at which time LC/MS analysis showed completion of the reaction. The solvent was removed under reduced pressure, and the residue was purified via flash chromatography (ISCO with 0-15% methanol/EtOAC as eluent) to afford the title compound (79 mg, 40% yield): ¹H NMR (400 MHz, MeOH-d₄) δ ppm 8.55 (dd, J=2.07, 0.35 Hz, 1H), 7.96 (dd, J=1.42 Hz, 1.26 Hz 1H), 7.89 (dd, J=8.41, 2.50 Hz, 1H), 7.46-7.52 (m, 3H), 6.55 (dd, J=8.70, 0.32 Hz, 1H), 6.27 (dd, J=7.58, 2.74 Hz, 1H), 6.04 (d, J=0.11 Hz, 1H), 5.21 (s, 2H), 3.74-3.78 (m, 1H), 3.59-3.72 (m, 2H), 3.41-3.45 (m, 1H), 3.20-3.24 (m, 1H) 2.57-2.65 (m, 1H), 2.25-2.37 (m, 1H), 1.63-2.03 (m, 6H), 1.09-1.36 (m, 5H); ES-LCMS m/z 480 (M+H)⁺.

### Example 25

### 4-{[(5-chloro-2-pyridinyl)methyl]oxy}-6′-[3-(cyclopentylamino)-1-pyrrolidinyl]-2H-1,3′-bipyridin-2-one

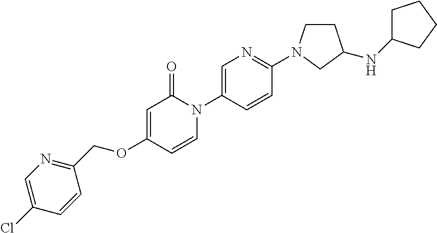

A mixture of 1-(5-bromo-2-pyridinyl)-3-pyrrolidinyl methanesulfonate (500 mg, 1.557 mmol) and cyclopentylamine (3 ml, excess) was sealed then allowed to heat to 120° C. and stir overnight. The reaction mixture was allowed to cool to room temperature then filtered through a pad of silica gel. Upon concentration of the filtrate, the product, 1-(5-bromo-2-pyridinyl)-N-cyclopentyl-3-pyrrolidinamine was collected as a light-brown oil (131 mg, 40%): ES-LCMS m/z 311 (M+H)⁺.

A mixture of the above 1-(5-bromo-2-pyridinyl)-N-cyclopentyl-3-pyrrolidinamine (131 mg, 0.423 mmol) and 4-{[(5-chloro-2-pyridinyl)methyl]oxy}-2(1H)-pyridinone (100 mg, 0.423 mmol) was allowed to stir and dissolve in 1,4-dioxane (10 mL). Next, NaI (127 mg, 0.845 mmol), K₂CO₃ (117 mg, 0.845 mmol), and CuI (32.2 mg, 0.169 mmol) were added and the reaction mixture flushed with dry nitrogen for 15 minutes. Trans-cyclohexane-1,2-diamine (24.5 mg, 0.169 mmol) was then added, and the reaction vessel was sealed. The reaction was heated to 130° C. and allowed to stir for 15 h. After cooling to 25° C., the solvent was removed under reduced pressure, and the residue purified via flash chromatography (ISCO with 0-15% methanol/EtOAc as eluent) to afford the title compound (79 mg, 40%) of: ¹H NMR (400 MHz, DMSO-d₆) δ ppm 8.65 (dd, J=1.35, 1.10 Hz, 1H), 7.95-8.01 (m, 1H), 7.57 (d, J=8.76, 1H), 7.52 (d, J=7.59 Hz, 1H), 7.43-7.46 (m, 1H), 6.46 (m, 1H), 6.46 (m, 1H), 6.09 (d, J=7.63 Hz, 1H), 5.91 (d, J=2.84 Hz, 1H), 5.21 (s, 2H), 3.57-3.63 (m, 1H), 3.46-3.54 (m, 1H), 3.28-3.38 (m, 4H), 3.06-3.18 (m, 2H) 2.08-2.17 (m, 1H), 1.71-1.84 (m, 3H), 1.55-1.66 (m, 2H), 1.42-1.50 (m, 2H); ES-LCMS m/z 466 (M+H)⁺.

### Example 26

### 4-{[(5-chloro-2-pyridinyl)methyl]oxy}-6′-[4-(pyrrolidin-1-yl)piperidinyl]-2H-1,3′-bipyridin-2-one

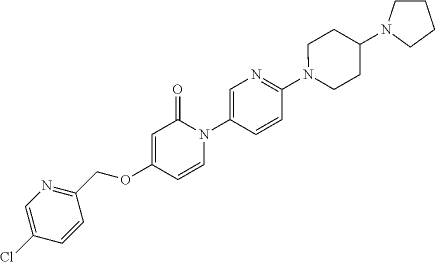

4-{[(5-chloro-2-pyridinyl)methyl]oxy}-6′-(fluoro)-2H-1,3′-bipyridin-2-one (100 mg, 0.30 mmol), 4-(pyrrolidin-1-yl)piperidine (51.2 mg, 0.33 mmol) and K₂CO₃ (125 mg, 0.906 mmol) were dissolved in DMF (2 mL), and the mixture was stirred at 110° C. for 18 h. After LCMS showed the stating material was consumed, the solvent was removed in vacuo to give the crude product, which was purified by preparative HPLC to afford 4-{[(5-chloro-2-pyridinyl)methyl]oxy}-6′-[4-(pyrrolidin-1-yl)piperidinyl]-2H-1,3′-bipyridin-2-one (7.55 mg, 5%): ¹H NMR (400 MHz, MeOH-d₄) δ ppm 8.48 (d, J=2.4 Hz, 1H), 7.99 (d, J=2.8 Hz, 1H), 7.82 (dd, J=4.4, 2.4 Hz, 1H), 7.51 (d, J=2.4 Hz, 1H), 7.50 (d, J=1.2 Hz, 1H), 7.45 (dd, 1H), 6.91 (d, J=8.8 Hz, 1H), 6.21 (dd, J=7.6, 2.8 Hz, 1H), 5.98 (d, J=2.8 Hz, 1H), 5.14 (s, 2H), 4.43 (d, 2H), 3.55-3.56 (m, 2H), 3.33 (m, 1H), 3.06-3.09 (m, 2H), 2.87-2.94 (m, 2H), 2.12-2.15 (m, 4H), 2.07-2.08 (m, 2H), 1.50-1.70 (m, 2H); LCMS m/z 466.3 (M+H)⁺.

### Example 27

### 4-{[(5-chloro-2-pyridinyl)methyl]oxy}-6′-(3-{[2-(methyloxy)ethyl]amino}-1-pyrrolidinyl)-2H-1,3′-bipyridin-2-one

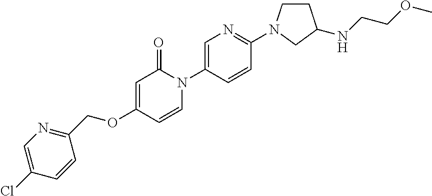

A mixture of 1-(5-bromo-2-pyridinyl)-3-pyrrolidinyl methanesulfonate (200 mg, 0.623 mmol) and 2-(methyloxy)ethanamine (5 ml, excess) was heated to 100° C. and allowed to stir overnight. The crude reaction mixture was filtered through a pad of silica gel, and the filtrate was concentrated to give 1-(5-bromo-2-pyridinyl)-N-[2-(methyloxy)ethyl]-3-pyrrolidinamine as a light-brown oil (177 mg, 85%): ES-LCMS m/z 301 (M+H).

A mixture of the above 1-(5-bromo-2-pyridinyl)-N-[2-(methyloxy)ethyl]-3-pyrrolidinamine (177 mg, 0.590 mmol) and 4-{[(5-chloro-2-pyridinyl)methyl]oxy}-2(1H)-pyridinone (140 mg, 0.590 mmol) was allowed to stir and dissolve in 1,4-dioxane (10 mL). NaI (177 mg, 1.179 mmol), K₂CO₃ (163 mg, 1.179 mmol), and CuI (44.9 mg, 0.236 mmol) were added, and the reaction mixture was flushed with dry nitrogen for 15 min. trans-Cyclohexane-1,2-diamine (33.5 mg, 0.236 mmol) was then added, and the reaction vessel was sealed. The reaction was heated to 130° C. and allowed to stir for 15 h. After cooling to 25° C., the solvent was removed under reduced pressure, and the residue purified by flash chromatography (ISCO with 0-15% methanol/EtOAC as eluent) to afford the title compound (34 mg, 25%): ¹H NMR (400 MHz, MeOH-d₄) δ ppm 8.55 (dd, J=2.10, 0.38 Hz, 1H), 7.96 (dd, J=2.34 Hz, 0.30 Hz 1H), 7.89 (dd, J=8.41, 2.50 Hz, 1H), 7.47-7.59 (m, 3H), 6.55 (dd, J=9.08, 0.32 Hz, 1H), 6.27 (dd, J=7.63, 2.74 Hz, 1H), 6.04 (d, J=2.69 Hz, 1H), 5.21 (s, 2H), 3.70-3.72 (m, 1H), 3.59-3.65 (m, 1H), 3.43-3.51 (m, 4H), 3.33 (s, 3H) 3.25-3.29 (m, 1H), 2.82-2.87 (m, 2H), 2.26-2.34 (m, 1H), 1.87-1.95 (m, 1H); ES-LCMS m/z 456 (M+H)⁺.

### Example 28

### 4-{[(5-chloro-2-pyridinyl)methyl]oxy}-6′-[3-(tetrahydro-2H-pyran-4-ylamino)-1-pyrrolidinyl]-2H-1,3′-bipyridin-2-one

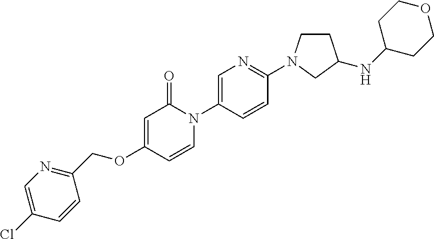

A mixture of 1-(5-bromo-2-pyridinyl)-3-pyrrolidinyl methanesulfonate (150 mg, 0.467 mmol) and tetrahydro-2H-pyran-4-amine (142 mg, 1.401 mmol) was dissolved in acetonitrile. The reaction vessel was sealed, heated to 120° C., and stirred overnight. The reaction mixture was allowed to cool to 25° C. then filtered through a pad of silica gel. Upon concentration of the filtrate, product 1-(5-bromo-2-pyridinyl)-N-(tetrahydro-2H-pyran-4-yl)-3-pyrrolidinamine, was collected as a brown oil (50 mg, 33%): ES-LCMS m/z 327 (M+H)⁺.

A mixture of the above 1-(5-bromo-2-pyridinyl)-N-(tetrahydro-2H-pyran-4-yl)-3-pyrrolidinamine (50 mg, 0.153 mmol) and 4-{[(5-chloro-2-pyridinyl)methyl]oxy}-2(1 pyridinone (36.3 mg, 0.153 mmol) was allowed to stir and dissolve in 1,4-dioxane (10 mL). Next, NaI (45.9 mg, 0.307 mmol), K₂CO₃ (42.4 mg, 0.307 mmol), and CuI (11.68 mg, 0.061 mmol) were added, and the reaction mixture flushed with dry nitrogen for 15 min. trans-Cyclohexane-1,2-diamine (8.7 mg, 0.061 mmol) was then added, and the reaction vessel sealed. The reaction was heated to 130° C. and stirred for 15 h. After cooling to 25° C., the solvent was removed under reduced pressure, and the residue purified by flash chromatography (ISCO with 0-15% methanol/EtOAC as eluent) to afford 29 the title compound mg (39% yield): ¹H NMR (400 MHz, DMSO-d₆) δ ppm 8.64 (dd, J=2.52, 0.12 Hz, 1H), 7.93-8.02 (m, 2H), 7.56-7.58 (m, 1H), 7.51-7.53 (m, 1H), 7.41-7.47 (m, 1H), 6.42-6.47 (m, 1H), 6.07-6.12 (m, 1H), 5.91 (d, J=2.79 Hz, 1H), 5.21 (s, 2H), 3.77-3.84 (m, 2H), 3.46-3.64 (m, 3H), 3.23-3.39 (m, 5H), 3.06-3.14 (m, 1H), 2.05-2.16 (m, 1H), 1.69-1.87 (m, 4H), 1.15-1.28 (m, 2H); ES-LCMS m/z 482 (M+H)⁺.

### Example 29

### 4-{[(5-chloro-2-pyridinyl)methyl]oxy}-6′-[3-(propylamino)-1-pyrrolidinyl]-2H-1,3′-bipyridin-2-one

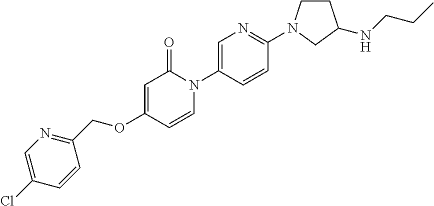

A mixture of 1-(5-bromo-2-pyridinyl)-3-pyrrolidinyl methanesulfonate (250 mg, 0.778 mmol) and 1-propanamine (230 mg, 3.89 mmol) was allowed to stir and dissolve in acetonitrile. The reaction vessel was sealed, heated to 120° C., and stirred overnight. The reaction mixture was allowed to cool to 25° C. and filtered through a pad of silica gel. Concentration of the filtrate yielded product 1-(5-bromo-2-pyridinyl)-N-propyl-3-pyrrolidinamine as a brown oil (96 mg, 43%): ES-LCMS m/z 286 (M+H)⁺.

A mixture of the above 1-(5-bromo-2-pyridinyl)-N-propyl-3-pyrrolidinamine (96 mg, 0.338 mmol) and 4-{[(5-chloro-2-pyridinyl)methyl]oxy}-2(1H)-pyridinone (80 mg, 0.338 mmol) was allowed to stir and dissolve in 1,4-dioxane (10 mL). NaI (101 mg, 0.676 mmol), K₂CO₃ (93 mg, 0.676 mmol), and CuI (25.7 mg, 0.135 mmol) were added, and the reaction mixture flushed with dry nitrogen for 15 min. trans-Cyclohexane-1,2-diamine (19.2 mg, 0.135 mmol) was then added, and the reaction vessel sealed. The reaction was heated to 130° C. and stirred for 15 h. The solvent was removed under reduced pressure, and the residue purified via flash chromatography (ISCO with 0-15% methanol/EtOAC as eluent) to afford the title compound (72 mg, 49% yield): ¹H NMR (400 MHz, DMSO-d₆) δ ppm 8.65 (d, J=0.15 Hz, 1H), 7.93-8.02 (m, 2H), 7.50-7.59 (m, 2H), 7.42-7.46 (m, 1H), 6.44 (d, J=9.00 Hz, 1H), 6.09 (dd, J=7.63, 2.79 Hz, 1H), 5.91 (d, J=0.10 Hz, 1H), 5.19 (s, 2H), 3.43-3.59 (m, 2H), 3.27-3.39 (m, 4H), 3.11-3.17 (m, 1H), 2.03-2.12 (m, 1H) 1.72-1.83 (m, 1H), 1.34-1.44 (m, 2H), 0.85 (t, 3H); ES-LCMS m/z 440 (M+H)⁺.

### Example 30

### 4-{[(5-chloro-2-pyridinyl)methyl]oxy}-6′-[3-(methoxycarbonylamino)-1-pyrrolidinyl]-2H-1,3′-bipyridin-2-one

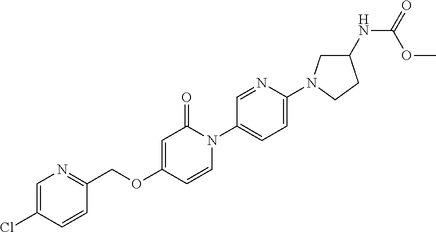

3-(Methoxycarbonylamino)pyrrolidine (62 mg, 0.3 mmol), 4-{[(5-chloro-2-pyridinyl)methyl]oxy}-6′-(fluoro)-2H-1,3′-bipyridin-2-one (100 mg, 0.3 mmol), and K₂CO₃ (166 mg, 1.21 mmol) were dissolved in DMF (2 mL). Then, the mixture was stirred at 110° C. for 18 h. After LCMS showed the stating material was consumed, the solvent was removed in vacuo to give the crude product, which was purified by preparative HPLC to afford the target molecule 4-{[(5-chloro-2-pyridinyl)methyl]oxy}-6′-[3-(methoxycarbonylamino)-1-pyrrolidinyl]-2H-1,3′-bipyridin-2-one (30 mg, 22%): ¹H NMR (400 MHz, MeOH-d₄) δ ppm 8.56 (d, J=2.00 Hz, 1H), 8.09 (d, J=2.00 Hz, 1H), 7.95 (dd, J=9.60, 2.40 Hz, 1H), 7.90 (dd, J=8.40, 2.40 Hz, 1H), 7.56 (t, J=8.00 Hz, 1H), 7.09 (d, J=9.60 Hz, 1H), 6.32 (dd, J=8.00, 2.40 Hz, 1H), 6.07 (d, J=2.80 Hz, 1H), 5.23 (s, 2H), 4.32-4.53 (m, 1H), 3.84-3.89 (m, 1H), 3.74-3.83 (m, 2H), 3.52-3.59 (m, 1H), 3.29 (s, 3H), 2.21-2.30 (m, 1H), 2.03-2.13 (m, 1H).

### Example 31

### 4-{[(5-chloro-2-pyridinyl)methyl]oxy}-6′-[4-(N-methyl-tert-butyloxycarbonylamino)-1-piperidinyl]-2H-1,3′-bipyridin-2-one

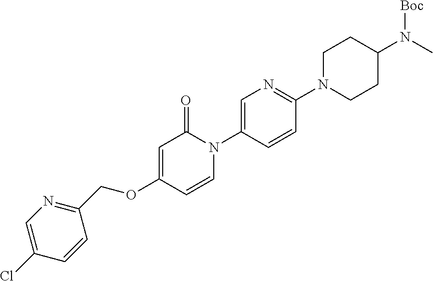

4-{[(5-chloro-2-pyridinyl)methyl]oxy}-6′-(fluoro)-2H-1,3′-bipyridin-2-one (50 mg, 0.15 mmol), 4-(N-methyl-tert-butyloxycarbonylamino)piperidine (35.53 mg, 0.17 mmol) and K₂CO₃ (41.66 mg, 0.30 mmol) was dissolved in DMF (2 mL) and the mixture was heated at 110° C. for 18 h. After LCMS showed the stating material was consumed, the solvent was removed in vacuo to give the crude product, which was purified by preparative HPLC to afford 4-{[(5-chloro-2-pyridinyl)methyl]oxy}-6′-[4-(N-methyl-tert-butyloxycarbonylamino)-1-piperidinyl]-2H-1,3′-bipyridin-2-one (10 mg, 18%): LCMS m/z 413 (M+H−100 [BOC])⁺.

### Example 32

### 4-{[(5-chloro-2-pyridinyl)methyl]oxy}-6′-[4-(N-methylamino)-1-piperidinyl]-2H-1,3′-bipyridin-2-one

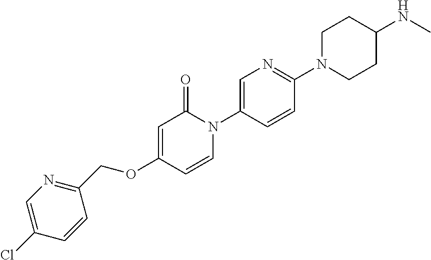

4-{[(5-chloro-2-pyridinyl)methyl]oxy}-6′-[4-(N-methyl-tert-butyloxycarbonylamino)-1-piperidinyl]-2H-1,3′-bipyridin-2-one (10 mg, 0.019 mmol) was dissolved in 4 N HCl/MeOH solution (10 mL), and the mixture was stirred at 0° C. for 0.5 h. After TLC showed the starting material was consumed, the solvent was removed in vacuo to give 4-{[(5-chloro-2-pyridinyl)methyl]oxy}-6′-[4-(N-methylamino)-1-piperidinyl]-2H-1,3′-bipyridin-2-one (5.4 mg, 71%): ¹H NMR (400 MHz, MeOH-d₄) δ ppm 8.52 (s, 1H), 8.09 (s, 1H), 7.88 (s, 1H), 7.86 (d, J=δ Hz, 1H), 7.53 (d, J=7.6 Hz, 2H), 7.39 (s, 1H), 6.27 (s, 1H), 6.01 (s, 1H), 5.18 (s, 2H), 4.34 (d, J=10.4 Hz, 2H), 3.32 (s, 1H), 3.22 (s, 2H), 2.68 (s, 3H), 2.25 (d, J=10 Hz, 2H), 1.73 (s, 2H); LCMS m/z 425 (M+H)⁺.

### Example 33

### 4-{[(5-chloro-2-pyridinyl)methyl]oxy}-6′-[3-(N,N-dimethylamino)-1-piperidinyl]-2H-1,3′-bipyridin-2-one

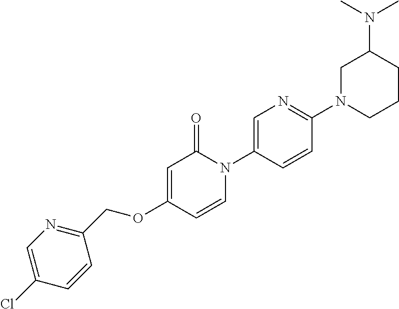

4-{[(5-chloro-2-pyridinyl)methyl]oxy}-6′-(fluoro)-2H-1,3′-bipyridin-2-one (50 mg, 0.15 mmol), 3-dimethylaminopiperidine (27.1 mg, 0.16 mmol) and K₂CO₃ (41.66 mg, 0.30 mmol) were dissolved in DMF (2 mL), and the mixture was heated at 110° C. for 18 h. After LCMS showed the stating material was consumed, the solvent was removed in vacuo to give the crude product, which was purified by TLC (CH₂Cl₂:MeOH/20:1) and preparative HPLC to afford 4-{[(5-chloro-2-pyridinyl)methyl]oxy}-6′-[3-(N,N-dimethylamino)-1-piperidinyl]-2H-1,3′-bipyridin-2-one (2.01 mg, 4%): ¹H NMR (400 MHz, MeOH-d₄) δ ppm 8.52 (s, 1H), 8.03 (s, 1H), 7.87 (d, J=10.8 Hz, 1H), 7.53 (d, J=9.2 Hz, 2H), 7.45 (d, J=7.6 Hz, 1H), 6.93 (d, J=8.8 Hz, 1H), 6.24 (d, J=10.4 Hz, 1H), 6.02 (s, 1H), 5.18 (s, 2H), 4.53 (d, J=13.6 Hz, 2H), 3.42 (m, 1H), 2.92 (t, J=12 Hz, 2H), 2.82 (s, 6H), 2.08 (d, J=12 Hz, 2H), 1.63-1.67 (m, 2H); LCMS m/z 439 (M+H)⁺.

### Examples 34 and 35

### Enantiomers 1 and 2 of 4-{[(5-chloro-2-pyridinyl)methyl]oxy}-6′-[3-(N-methylacetamido)-1-pyrrolidinyl]-2H-1,3′-bipyridin-2-one

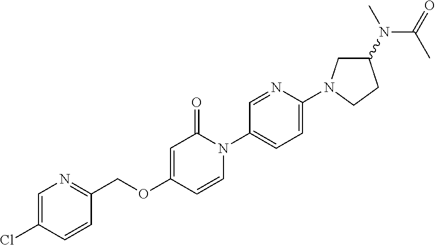

4-{[(5-chloro-2-pyridinyl)methyl]oxy}-6′-(fluoro)-2H-1,3′-bipyridin-2-one (300 mg, 0.91 mmol), 3-(N-methylacetamido)pyrrolidine (193 mg, 1.36 mmol) and K₂CO₃ (250 mg, 1.81 mmol) were dissolved in DMF (6 mL), and the mixture was heated at 110° C. for 12 h. After LCMS showed the stating material was consumed, the solvent was removed in vacuo to give the crude product, which was purified by chiral preparative HPLC to afford:

Enantiomer 1 (Retention Time=13.33 min) of 4-{[(5-chloro-2-pyridinyl)methyl]oxy}-6′-[3-(N-methylacetamido)-1-pyrrolidinyl]-2H-1,3′-bipyridin-2-one (17.64 mg, 11%): ¹H NMR (400 MHz, MeOH-d₄) δ ppm 8.57 (d, J=2.10 Hz, 1H), 8.02 (d, J=2.40 Hz, 1H), 7.93 (m, 1H), 7.50-7.59 (m, 3H), 6.61 (m, 1H), 6.30 (m, 1H), 6.06 (s, 1H), 5.23 (s, 2H), 3.64 (m, 2H), 3.50 (m, 2H), 3.02 (s, 3H), 2.21 (s, 2H), 2.13 (s, 2H), 1.13-1.30 (m, 2H).

Enantiomer 2 (Retention Time=15.24 min) of 4-{[(5-chloro-2-pyridinyl)methyl]oxy}-6′-[3-(N-methylacetamido)-1-pyrrolidinyl]-2H-1,3′-bipyridin-2-one (15.76 mg, 10%): ¹H NMR (400 MHz, MeOH-d₄) δ ppm 8.57 (d, J=2.10 Hz, 1H), 8.02 (d, J=2.40 Hz, 1H), 7.93 (m, 1H), 7.50-7.59 (m, 3H), 6.61 (m, 1H), 6.30 (m, 1H), 6.06 (s, 1H), 5.23 (s, 2H), 3.64 (m, 2H), 3.50 (m, 2H), 3.02 (s, 3H), 2.21 (s, 2H), 2.13 (s, 2H), 1.13-1.30 (m, 2H).

### Chiral SFC (Supercritical Fluid Chromatography) Separation Conditions

### Instrument: Berger MultiGram™ SFC, Mettler Toledo Co, Ltd

### Column: ChiralPak OJ, 5 μm, Daicel Chemical Industries, Ltd 250×20 mm I.D.

Mobile phase: A: Supercritical CO₂, B: MeOH (0.05% DEA), A:B=80:20 at 50 mL/min.

### Column Temp: 38° C.; Nozzle Pressure: 100 Bar; Nozzle Temp: 60° C.; Evaporator Temp: 20° C.; Trimmer Temp: 25° C.; Detection Wavelength: 220 nm.

MCHR1 pIC₅₀ Determination FLIPR™ Assay: Frozen U2OS cells were rapidly thawed in a 37° C. water bath 24 hours prior to assay. The cells were counted, diluted to appropriate concentration, and mixed with 0.53% v/v of human MCHR1 and 0.13% v/v Gqi5 BacMam virus stocks for transduction of the receptor. 50 uL of this cell suspension was plated utilizing a Combi Multidrop (Thermo) at a concentration of 15,000 cells/well in a black 384-well clear bottom plate (Greiner) in DMEM/F12 media containing 10% FBS and stored at 37° C. overnight. Compounds to be profiled were prepared by making a stock solution at 3×10⁻³M in 100% DMSO. The stock solutions were serially diluted 1:4 in 100% DMSO using a Beckman Biomek FX as 11 point curves in singlicate. In a polypropylene 384-well plate, 2 uL of the compound dilution was pipetted using a BiomekFX. The compound plate was diluted by adding 40 uL of load buffer to the plate and gently shaken. At the time of the assay, the media was removed from the cell plate by aspiration, followed by the addition of 20 uL of load buffer (Calcium Plus Kit, MDC) using a Matrix wellmate. Following a one hour incubation at 37° C., 10 uL of compound was added to the plates via the FLIPR™ instrument. The plates were incubated at 37° C. for 30 minutes along with an MCH peptide agonist challenge plate. On the FLIPR™, a basal response was collected over 10 seconds followed by the addition of 10 μL of MCH challenge made at the 4×EC₅₀ concentration in load buffer. Data was collected over 4 minutes and subjected to a nonlinear regression analysis curve fitting program to generate MCHR1 pIC₅₀s.

MCHR1 pCI₅₀ Determination Reporter Gene Assay: The assay consists of cells plated at ten thousand cells/well in DMEM/F12, 5% FBS, 2 mM l-glutamine in black 384-well assay plates. The day after plating, the media was removed by aspiration sixteen hours prior to assay, followed by the addition of 50 uL of media without serum to reduce background signal noise. Compounds were prepared by making a stock solution at 3×10⁻³M. The stock solutions were serially diluted 1:4 in 100% DMSO using a Beckman Biomek FX as 11 point curves in singlicate. On the day of the assay, compounds (0.5 uL) were pipetted into the assay plate using a BeckmanFX. Following incubation for 45 minutes at 37° C., a 4×EC₅₀ concentration of MCH (MCH R1) or thrombin (host) was added to the plate allowing for appropriate controls. The plates were then incubated under the same conditions for five hours. Under subdued light conditions, the compound/assay solution was removed by aspiration from the plates, followed by the addition of 20 uL of a 1:1 solution containing SteadyGlo™ and Dulbecco's Phosphate Buffered Saline with 1 mM CaCl₂ and 1 mM MgCl₂. Plates were sealed with self-adhesive clear plate seals and wiped with a static free dryer sheet to reduce false counts due to static charge. The amount of luciferase generated was quantified in a Viewlux (Perkin Elmer) with a 2 second per well count time.

Although specific embodiments of the present invention are herein illustrated and described in detail, the invention is not limited thereto. The above-detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A compound of Formula I, 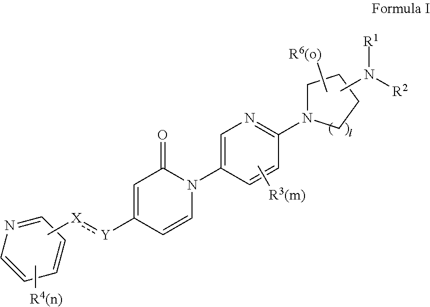
or salt thereof, wherein:
X and Y are independently selected from the group consisting of —O—, —CH₂—, and ═CH—, with the proviso that X and Y are not both —O—;
— is optionally a bond to form a double bond;
R¹ is selected from the group consisting of (i) hydrogen, (ii) substituted or unsubstituted, straight or branched C₁₋₆alkyl, and (iii) substituted or unsubstituted C₃₋₆cycloalkyl;
R² is selected from the group consisting of (i) hydrogen, (ii) substituted or unsubstituted, straight or branched C₁₋₆alkyl, (iii) —C(O)NH₂, (iv) —C(O)R⁵, (v) —SO₂R⁵, and (vi) C(O)OR¹;
or R¹ and R² together with the nitrogen to which they are attached to form a heterocycle, and said heterocycle is optionally substituted with one, two, or three R⁵ groups;
wherein each R⁵ independently is selected from the group consisting of (i) hydroxy, (ii) unsubstituted or substituted C₁₋₃alkoxy, (iii) unsubstituted or substituted, straight or branched C₁₋₆alkyl, and (iv) unsubstituted or substituted C₃₋₆cycloalkyl;
each R³ and R⁴ independently is selected from the group consisting of H, F, Cl, CF₃, CH₃, CH₂CH₃, CH₂CF₃, cyclopropyl, OMe, OEt, OiPr, O-cyclopropyl, OCF₃, OCH₂CF₃, CN, NMe₂, N-pyrrolidinyl, N-morpholinyl, and acetyl;
R⁶ is selected from the group consisting of (i) hydrogen, (ii) substituted or unsubstituted, straight or branched C₁₋₆alkyl, and (iii) substituted or unsubstituted C₃₋₆cycloalkyl;
l is 0, 1, or 2;
m is 0, 1, 2, or 3;
n is 0, 1, 2, or 3; and
o is 0, 1, 2, or 3.

2. The compound of claim 1 or salt thereof wherein X and Y are joined by a single bond.

3. The compound of claim or salt thereof wherein R¹ is selected from the group consisting of substituted or unsubstituted C₁₋₆alkyl and substituted or unsubstituted C₃₋₆cycloalkyl, and R² is H.

4. The compound of claim 3 or salt thereof wherein R¹ is a substituted C₁₋₆alkyl or a substituted C₃₋₆cycloalkyl.

5. The compound of claim 4 or salt thereof wherein said substituted C₁₋₆alkyl or substituted C₃₋₆ cycloalkyl is substituted with 1 to 6 fluorines.

6. The compound of claim 1 or salt thereof wherein R² is a substituted C₁₋₆alkyl.

7. The compound of claim 6 or salt thereof wherein said C₁₋₆alkyl is substituted with 1 to 6 fluorines.

8. The compound of claim 1 or salt wherein R¹ and R² are each methyl.

9. The compound of claim 1 or salt thereof wherein R¹ and R² are joined together with the nitrogen to which they are attached to form a pyrrolidinyl or a morpholinyl group.

10. The compound of claim 1 or salt thereof wherein R¹ and R² are joined together with the nitrogen to which they are attached to form a substituted or unsubstituted heterocycle.

11. The compound of claim 10 or salt thereof wherein said heterocycle is substituted with one to three R⁵ groups.

12. The compound of claim 11 or salt thereof wherein said R⁵ is selected from the group consisting of substituted C₁₋₃alkoxy, substituted C₁₋₆alkyl, and substituted C₃₋₆cycloalkyl.

13. The compound of claim 12 or salt thereof wherein said substituted C₁₋₃alkoxy, substituted C₁₋₆alkyl, and substituted C₃₋₆cycloalkyl is substituted with 1 to 6 fluorines.

14. The compound of claim 1 or salt thereof wherein said l is 1 or 2.

15. The compound of claim 14 or salt thereof wherein said l is 1.

16. The compound of claim 1 or salt thereof wherein m is 0.

17. The compound of claim 1 or salt thereof wherein n is 0, 1, or 2.

18. The compound of claim 1 or salt thereof wherein o is 0, 1, or 2.

19. The compound of claim 1 or salt thereof wherein said compound is selected from the group consisting of
4-{[(5-chloro-2-pyridinyl)methyl]oxy}-6′-[3-(ethylamino)-1-pyrrolidinyl]-2H-1,3′-bipyridin-2-one;
4-{[(5-chloro-2-pyridinyl)methyl]oxy}-6′-[3-(methylamino)-1-pyrrolidinyl]-2H-1,3′-bipyridin-2-one;
4-{[(5-chloro-2-pyridinyl)methyl]oxy}-6′-[3-(dimethylamino)-1-pyrrolidinyl]-2H-1,3′-bipyridin-2-one;
4-{[(5-chloro-2-pyridinyl)methyl]oxy}-6′-[3-(dimethylamino)-1-pyrrolidinyl]-5′-methyl-2H-1,3′-bipyridin-2-one;
4-{[(5-chloro-2-pyridinyl)methyl]oxy}-6′-[3-(propylamino)-1-pyrrolidinyl]-2H-1,3′-bipyridin-2-one;
4-{[(5-chloro-2-pyridinyl)methyl]oxy}-6′-{3-[ethyl(methyl)amino]-1-pyrrolidinyl}-2H-1,3′-bipyridin-2-one;
4-{[(5-chloro-2-pyridinyl)methyl]oxy}-6′-{3-[methyl(1-methylethyl)amino]-1-pyrrolidinyl}-2H-1,3′-bipyridin-2-one;
4-{[(5-chloro-2-pyridinyl)methyl]oxy}-6′-[3-(cyclohexylamino)-1-pyrrolidinyl]-2H-1,3′-bipyridin-2-one;
4-{[(5-chloro-2-pyridinyl)methyl]oxy}-6′-[3-(cyclopentylamino)-1-pyrrolidinyl]-2H-1,3′-bipyridin-2-one;
4-{[(5-chloro-2-pyridinyl)methyl]oxy}-6′-(3-{[2-(methyloxy)ethyl]amino}-1-pyrrolidinyl)-2H-1,3′-bipyridin-2-one;
4-{[(5-chloro-2-pyridinyl)methyl]oxy}-6′-[3-(tetrahydro-2H-pyran-4-ylamino)-1-pyrrolidinyl]-2H-1,3′-bipyridin-2-one;
4-{[(5-chloro-2-pyridinyl)methyl]oxy}-6′-(1,3′-bipyrrolidin-1′-yl)-2H-1,3′-bipyridin-2-one;
4-{[(5-chloro-2-pyridinyl)methyl]oxy}-6′-[3-(4-morpholinyl)-1-pyrrolidinyl]-2H-1,3′-bipyridin-2-one;
4-{[(5-chloro-2-pyridinyl)methyl]oxy}-6′-[3-(amino)-1-pyrrolidinyl]-2H-1,3′-bipyridin-2-one;
4-{[(5-chloro-2-pyridinyl)methyl]oxy}-6′-[3-(methoxycarbonylamino)-1-pyrrolidinyl]-2H-1,3′-bipyridin-2-one;
4-{[(5-chloro-2-pyridinyl)methyl]oxy}-6′-[4-(N-methylamino)-1-piperidinyl]-2H-1,3′-bipyridin-2-one;
4-{[(5-chloro-2-pyridinyl)methyl]oxy}-6′-[3-(N-methylacetamido)-1-pyrrolidinyl]-2H-1,3′-bipyridin-2-one;
4-{[(5-chloro-2-pyridinyl)methyl]oxy}-6′-[3-(N-methylamino)-4-methyl-1-pyrrolidinyl]-2H-1,3′-bipyridin-2-one;
4-{[(5-fluoro-2-pyridinyl)methyl]oxy}-6′-[3-(dimethylamino)-1-pyrrolidinyl]-2H-1,3′-bipyridin-2-one;
4-{[(2-pyridinyl)methyl]oxy}-6′-[3-(dimethylamino)-1-pyrrolidinyl]-2H-1,3′-bipyridin-2-one.

20. The compound of claim 19 or salt thereof wherein said compound is selected from the group consisting of
4-{[(5-chloro-2-pyridinyl)methyl]oxy}-6′-[3-(ethylamino)-1-pyrrolidinyl]-2H-1,3′-bipyridin-2-one;
4-{[(5-chloro-2-pyridinyl)methyl]oxy}-6′-[3-(methylamino)-1-pyrrolidinyl]-2H-1,3′-bipyridin-2-one;
4-{[(5-chloro-2-pyridinyl)methyl]oxy}-6′-[3-(dimethylamino)-1-pyrrolidinyl]-2H-1,3′-bipyridin-2-one;
4-{[(5-chloro-2-pyridinyl)methyl]oxy}-6′-[3-(dimethylamino)-1-pyrrolidinyl]-5′-methyl-2H-1,3′-bipyridin-2-one;
4-{[(5-chloro-2-pyridinyl)methyl]oxy}-6′-[3-(propylamino)-1-pyrrolidinyl]-2H-1,3′-bipyridin-2-one;
4-{[(5-chloro-2-pyridinyl)methyl]oxy}-6′-{3-[ethyl(methyl)amino]-1-pyrrolidinyl}-2H-1,3′-bipyridin-2-one;
4-{[(5-chloro-2-pyridinyl)methyl]oxy}-6′-{3-[methyl(1-methylethyl)amino]-1-pyrrolidinyl}-2H-1,3′-bipyridin-2-one;
4-{[(5-chloro-2-pyridinyl)methyl]oxy}-6′-[3-(cyclohexylamino)-1-pyrrolidinyl]-2H-1,3′-bipyridin-2-one;
4-{[(5-chloro-2-pyridinyl)methyl]oxy}-6′-[3-(cyclopentylamino)-1-pyrrolidinyl]-2H-1,3′-bipyridin-2-one;
4-{[(5-chloro-2-pyridinyl)methyl]oxy}-6′-(3-{[2-(methyloxy)ethyl]amino}-1-pyrrolidinyl)-2H-1,3′-bipyridin-2-one;
4-{[(5-chloro-2-pyridinyl)methyl]oxy}-6′-[3-(tetrahydro-2H-pyran-4-ylamino)-1-pyrrolidinyl]-2H-1,3′-bipyridin-2-one;
4-{[(5-chloro-2-pyridinyl)methyl]oxy}-6′-(1,3′-bipyrrolidin-1′-yl)-2H-1,3′-bipyridin-2-one.

21. The compound of claim 1 or salt thereof or a pharmaceutical composition thereof in combination with at least one other anti-obesity drug or anti-diabetic drug.

22. A pharmaceutical composition comprising a compound of claim 1 or salt thereof.

23. A pharmaceutical composition comprising a compound of claim 1 or salt thereof and one or more excipients.

24. A method of treatment comprising the administering to a mammal a pharmaceutical composition comprising a compound of claim 1 or a pharmaceutically acceptable salt thereof and at least one excipient, wherein said treatment is for obesity, diabetes, hypertension, depression, anxiety, drug addiction, substance addiction, or a combination thereof.

25. The method of claim 24 wherein said treatment is for obesity, diabetes, or both.

26. The method of claim 24 wherein said mammal is a human.

27-30. (canceled)

31. A process for making a compound of claim 1 or salt thereof comprising
reacting substituted pyridone intermediate (D) with 2-aminopyridine intermediate (E) to provide 2-aminopyridine (F).

32. A process for making a compound of claim 1 or salt thereof comprising the steps of
(i) heating substituted 2-halo-5-bromopyridines with 3-hydroxypyrrolidine in the presence of base to provide hydroxypyrrolidine intermediate (G);
(ii) forming mesylate intermediate (H);
(iii) displacing the mesylate group with substituted amine to provide substituted 2-aminopyridine intermediate (E); and
(iv) copper-mediated coupling of the substituted 2-aminopyridine intermediate (E) with substituted pyridone intermediate (D) to provide 2-aminopyridine-pyridone (F).

33. A process for making a compound of claim 1 or salt thereof comprising the steps of
(i) treating substituted 2-halo-5-bromopyridine with substituted piperidine, pyrrolidine or azetidine in the presence of base to provide substituted aminopyridine intermediate (E); and
(ii) copper-mediated coupling of substituted aminopyridine intermediate (E) with substituted pyridone intermediate (D) to provide 2-aminopyridine-pyridone (F).

34. A process for making a compound of claim 1 or salt thereof comprising the steps of
(i) oxidizing methylisonicotinate to its N-oxide;
(ii) treating said N-oxide with acetic anhydride in methanol to provide methyl 2-oxo-1,2-dihydro-4-pyridinecarboxylate;
(iii) reducing the pyridinecarboxylate ester with LiBH₄ followed by protection of the primary alcohol as the TBDMS ether to provide intermediate (I);
(iv) copper-mediated coupling of 2-aminopyridine intermediate (E) with intermediate (I) to provide substituted intermediate (J);
(v) acid-catalyzed removal of the silyl protecting group of intermediate (J) followed by subjection to a Mitsunobu reaction with substituted phenol to provide 2-aminopyridine-pyridone (F).
